(19) United States
(12) Patent Application Publication
(10) Pub. No.: US 2013/0034537 A1
(43) Pub. Date: Feb. 7, 2013

(54) SIMULTANEOUS CO-TREATMENT OF PHYSICAL AND MENTAL SYMPTOMS RELATED TO SEVERE MENTAL DISORDERS BY A SPECIALLY FERMENTED SOY FORMULATION (FSWW08)

(71) Applicants: Anca-Gabriela GOCAN, Vienna (AT); Uwe ROHR, Vienna (AT); Walter WAINRIGHT, Woodinville, WA (US)

(72) Inventors: Anca-Gabriela GOCAN, Vienna (AT); Uwe ROHR, Vienna (AT); Walter WAINRIGHT, Woodinville, WA (US)

(21) Appl. No.: 13/315,258

(22) Filed: Dec. 8, 2011

Related U.S. Application Data
(60) Provisional application No. 61/503,787, filed on Jul. 1, 2011.
(60) Provisional application No. 61/421,088, filed on Dec. 8, 2010.

(51) Int. Cl. A61K 31/352; A61P 25/00; A61P 25/18; A61P 1/04; A61P 31/12; A61P 25/22; A61P 1/00; A61K 36/185; A61P 3/00

(52) U.S. Cl. 424/115; 514/456

(57) ABSTRACT

A method of treating severe mental diseases and related disorders or conditions includes administering to a patient in need thereof an effective amount of a composition including isoflavones and Bowman Birk Inhibitory Factor (BBIF). Preferably, the composition is a fermented soy composition. Preferably, one first diagnoses a patient with a particular mental disease or disorder, and then prescribes the composition as a treatment for the particular mental disease or disorder. A patient is preferably given daily a dosage of 3-10 mg of isoflavones and 5-10 Cl (chymotrypsin inhibitor) of BBIF per kg of body weight, more preferably a dosage of 5-9 mg of isoflavones and 5-10 CCl of BBIF per kg of body weight, and most preferably a dosage of 5-10 mg of isoflavones and 5-10 CCl of BBIF per kg of body weight. Treatment preferably lasts 7 days-14 days, or until all symptoms disappear.

### CROSS-REFERENCE TO RELATED APPLICATIONS

This is a nonprovisional patent application of U.S. Provisional Patent Application Ser. No. 61/503,787, filed 1 Jul. 2011, which is hereby incorporated herein by reference.

This is a nonprovisional patent application of U.S. Provisional Patent Application Ser. No. 61/421,088, filed 8 Dec. 2010, which is hereby incorporated herein by reference.

Priority of U.S. Provisional Patent Application Ser. No. 61/503,787, filed 1 Jul. 2011 and U.S. Provisional Patent Application Ser. No. 61/421,088, filed 8 Dec. 2010, which are incorporated herein by reference, is hereby claimed.

### STATEMENT REGARDING FEDERALLY SPONSORED RESEARCH OR DEVELOPMENT

Not applicable

### REFERENCE TO A “MICROFICHE APPENDIX”

Not applicable

### BACKGROUND OF THE INVENTION

### Field of Invention

The present invention relates to simultaneous treatment of immune diseases (infections and inflammation) and metabolic diseases, blood hematology and steroidal hormone cascade in patients suffering from severe mental disorders, by a specially fermented soy formulation (FSWW08) containing isoflavones in concentration similar to baby soy nutrition formula (300 to 1000 mg preferably 500 to 1000 mg), and a combination with the Bowman Birk Inhibitors factors in concentrations higher than 100 CCl, preferably in the range of 500-1000 CCl.

The application is a cotreatment in the field of mental disease and their always accompanying life threatening diseases also, like viral infections, altered blood hematology, metabolic diseases, by reducing the stress hormones DHEA and pregnenolone, as well as increase androstenediol, androstanediol testosterone, with a specially fermented soy product (FSWW08). Besides soy isoflavones, the formulation contained particularly the protease inhibitor BBI, the Bowan Birk Inhibitory Factor, which has strong immuno-modulating effects. Also inflammatory Th1-cytokines are reduced in these patients. Soy isoflavones in concentration in that formulations similar to soy baby nutrition formula, which did not show immunological effects in babies and is used for more than 100 years, higher than 300 mg, preferably 500 to 1000 mg, do reduce anxiety, stress, severe mental disease, as well as show strong immunity improving effects.

### Primary Class: 514/282

### Summary

Severe mental Diseases affect many persons either over a short period of life or even throughout life. It is wrongly assumed by the public, even by the many medical doctors, that mental diseases are purely “mental diseases”, because the physical basis of these diseases have not been understood. Severe mental diseases are always accompanied by poor blood lipids causing life threatening disease, viral infections which are all treatment resistant with conventional medicine. So there is no treatment for PTSD nor their accompanying physical disease. For the first time ever it was possible to treat persons with severe mental diseases completely, not only the mental symptoms, but physical symptoms as well, like disturbed blood formation, viral infections, life threatening increase of triglycerides, to name only key markers, which are found in all persons suffering from severe mental diseases. Everyone involved in reviewing this patent application needs to understand that there are no medication treating triglycerides or viral infections in these patients, so that the life span of these patients is reduced about 20 to 30 years. Therefore this patent claims that both, mental and physical markers of mental diseases are improved, what improves the disease completely. It is the key finding of this patent that severe mental diseases (SMD) like schizophrenia, Bipolar disorder, post-traumatic stress disorder, postnatal depression, and compulsive disorder, anxiety attacks, Tourette Syndrome, and attention deficit disorders, (ADHD) share similar biological denominators, and that when these denominators are corrected, the mental diseases can be corrected. Diagnostic and Statistical Manual of Mental Diseases (DSM) diagnoses mental diseases, the main finding of this patent is that they can be treated with similar medications.

The hormonal-cascade-imbalances of the HPA axis in SMD are enormous, however, their patterns are very similar. Also severe mental diseases always co-exist with SMD. It is known to the experts in the field that severe mental diseases share many hormonal similarities as well as genetic similarities and it has been speculated for a long time that they may have a common denominator. In SMD patients the steroidal hormones in the upper part of the hormonal bio-synthesis pathway are increased and at the same time are decreased in the lower part of the hormonal bio-synthesis. SMD are not isolated diseases. Metabolic disorders and immunity disorders, which are accompanying SMD, cause a reduction in life spans of 25-30 years. The accompanying disorders, like increased triglycerides and blood lipids, are normally treatment resistant to known drugs. It would be highly desirable for the physician to have a treatment, which corrects both, mental as well as physical diseases. It is a major finding in this invention that normalizing of steroidal hormonal balance corrects mental as well as physical diseases. Current treatment improves some physiologic parameters in SMD, however they frequently worsen many parameters, like anti-psychotics increase blood lipids, which increase the arteriosclerotic risk even more. The key finding of this invention is that fermented soy (FSWW08) in doses similar to soy baby nutrition formulas can correct the mental diseases and almost all physiologic accompanying disturbances as well. Doses are not only defined as isoflavones but include the BOWMAN BIRK INHIBITORY FACTOR. Most importantly blood lipids are normalized, increased white blood counts reduced, and the water retention is reduced that often cause life threatening breathing problems and lung edema. The correction of immune disturbances is so pronounced, that prevention of Lupus is possible. A key finding of this patent is that the upper synthesized hormones derived from cholesterol, the stress hormones DHEA and pregnenolone, are decreased by increasing their metabolism. This results in higher hormone levels in the lower areas of the bio-synthesis pathway in increased levels like testosterone, estradiol, androstenediol and androstanediol, as well as cortisol.

The hormones improving the immune system, and well-being are derived from stress hormones. Since no side effects were observed, the treatment allows, for the first time ever, prevention and treatment of SMD and their accompanying diseases like Lupus.

It is known to the expert, that in animal experiments soy can decrease the hormone metabolism of pregnenolone to aldosterone and DHEA is increased. This was also seen in human studies with limited participants, if the applied soy dose is low. This invention shows for the first time ever, that fermented soy formulation (FSWW08) decreases both stress hormones DHEA and pregnenolone and simultaneously increases functional hormones. The standard technological medical procedure to achieve the increase of hormones in patients is by external delivery. This is coined as hormone replacement therapy. This invention shows for the first time ever that missing or low levels of steroidal hormones in a patient can be increased by metabolizing existing hormones in the body that are in the normal range, elevated above normal, which are increased and related to stress. The precurser hormones can cause stress and edema. The decrease of precurser hormones (DHEA, pregnenolone) leads to increased biosynthesis of testosterone, ADIOL, Estradiol, and cortisol. The effect is not limited to FSWW08, but may be facilitated by red clover, alfalfa, and other isoflavone containing plants, or synthetic preparations, that are applied in the right daily dose. It is a key finding of this patent that HPA axis disturbances in pregnancy and in severe mental disturbances show similarity. Pregnancy is regulated by the HPA axis, and severe disturbances result in mental disturbances like depression and postnatal depression. Female veteran soldiers often suffer from severe mental diseases like Schizophrenia, Bipolar disorders, although they were healthy until they became pregnant. Mechanisms controlling both a) the rupture of the uterus membrane during pregnancy can cause decrease feelings of well being and miscarriage and are also b) causing stroke and infarction in older men and women or c) severe mental diseases. This invention recognizes, for the first time ever, that disturbances of the HPA axis always causes not only severe mental but also severe physical diseases and both have to be corrected simultaneously. In addition, this invention shows how the use of isoflavones, in correct doses, corrects both the physical and severe mental diseases caused by hormonal imbalances resulting from HPA axis disturbances.

A method of treating severe mental diseases and related disorders or conditions includes administering to a patient in need thereof an effective amount of a composition including isoflavones and BBIF. Preferably, the composition is a fermented soy composition. Preferably, one first diagnoses a patient with a particular mental disease or disorder, and then prescribes the composition as a treatment for the particular mental disease or disorder. A patient is preferably given daily a dosage of 3-10 mg of isoflavones and 5-10 Cl (chymotrypsin inhibitor) of Bowman Birk Inhibitory Factor per kg of body weight, more preferably a dosage of 5-9 mg of isoflavones and 5-10 CCl of Bowman Birk Inhibitory Factor per kg of body weight, and most preferably a dosage of 5-10 mg of isoflavones and 5-10 CCl of Bowman Birk Inhibitory Factor per kg of body weight. The doses of BBIC. Bowman Birk Inhibitory Concentrate, are measured in Cl units, as described in detail elsewhere (Kennedy et al., 1993b); BBIC contains 100 mg/g Cl activity, and, at most, 40 mg/g TI activity. FSWW08 contains the highest amount of BBIC measured in a product. This can be achieved by giving a patient daily 50 ml-200 ml of FSWW08, or about 1-3 ml of FSWW08 per kg of body weight. Treatment preferably lasts 7 days-14 days, or until all symptoms disappear.

The preferred fermented soy formulation is SOY-IQ, also referred to herein as FSWW08. However, other fermented soy formulations can be used, as long as the recommended amounts of isoflavones and Bowman Birk Inhibitory Factor are provided to the patients.

### DESCRIPTION

### Technical Field

This disclosure is in the medical field and in particular to the fields of psychology and psychiatry, as well as immunity, metabolic disease, health, diet and nutrition.

### Background

Over the last six decades, the treatment of schizophrenia has focused primarily on interactions at monoamine neurotransmitter receptor sites, including those for dopamine and serotonin[1-4]. While first-generation antipsychotics demonstrate antagonism at the dopamine 2 receptor, newer atypical agents involve multiple receptors at various neurotransmitter sites[1-4]. Despite the advent of these newer agents, the treatment of schizophrenia continues to elude clinicians, perhaps owing to a lack of information about the factors contributing to the development of the disease. While the etiology is complex and not yet fully delineated, it is recommended that treating clinicians should look beyond neurotransmitters and other potential factors involved in the pathogenesis of schizophrenia: It is more and more recognized that autoimmunity may be the primary origin of the disease development in at least a subset of patients[1-4]. Recent investigation suggests a strong relationship between immunological effects and the pathophysiology of schizophrenia. Many clinicians suggest therefore that treatment of immune dysfunction in the development of schizophrenia and demand future directions for the field[1-4]. For the first time ever, a fermented soy product did not only improve mental and biological disorders of severely metal diseases like bipolar disorders and schizophrenia, but also accompanying immune diseases like inflammations and infections supporting the evidence that schizophrenia may be caused by a disturbance of auto-immunity. This effect is achieved by interplay of correcting neuro-active and immunologically active and sexual hormones, T1 and Th2 cytokines, and hormones of the HPA-axis. For the first time ever, a formulation corrects infections and inflammation simultaneously. Until now, glucocorticoids focus only on correcting anti-inflammatory diseases. The simultaneous inflammatory and anti-infective activity is obtained by increasing physiological levels of androstenes (androstenediol, a metabolite of DHEA and androstanediol, a metabolite of Testosterone via DHT), which are the only known sexual steroids, which can improve simultaneously inflammation and infection. For the first time ever, fermented soy improves blood lipids of patients suffering from severe mental diseases besides improving wellbeing, which are held responsible to reduce life expectancy of 25 to 30 years. The finding is important, since current anti-psychotics worsens blood lipids, increasing the risk succumb to diabetes, stroke or infarction even more[5,6]. It is the basis finding of this patent, that severe mental diseases like schizophrenia, bipolar diseases, compulsive disorders, are caused by disturbances of the hormonal, cytokine, and neuro-hormonal level and may be caused by similar disturbances. Although mental diseases are different on the genetic level[7] and are categorized by psychiatrist into different diseases[8,9] they share side effects like metabolic disorders, reduction of the immune system causing inflammation and infections. Sharing similar side effects is an important indication that the etiology of severe mental diseases may be similar. Mental disorders are common in the United States and internationally. An estimated 26.2 percent of Americans ages 18 and older—about one in four adults—suffer from a diagnosable mental disorder in a given year[8]. When applied to the 2004 U.S. Census residential population estimate for ages 18 and older, this figure translates to 57.7 million people[9]. Even though mental disorders are widespread in the population, the main burden of illness is concentrated in a much smaller proportion—about 6 percent, or 1 in 17—that suffer from a serious mental illness. In addition, mental disorders are the leading cause of disability in the U.S. and Canada[9]. Many people suffer from more than one mental disorder at a given time. Nearly half (45 percent) of those with any mental disorder meet criteria for 2 or more disorders, with severity strongly related to comorbidity [9]. That many patients suffer from multiple mental diseases supports the assumption, that they share similar biological roots.

Accordingly to the NATIONAL INSTITUTE OF MENTAL HEALTH, Psychiatrists have provided their needs to categorize mental disease, as follows

Mood disorders are divided into
depression and
bipolar disorders,

### Schizophrenia

### Anxiety Disorders

Anxiety disorders include panic disorder, obsessive-compulsive disorder, post-traumatic stress disorder, generalized anxiety disorder, and phobias (social phobia, agoraphobia, and specific phobia).

Attention Deficit Hyperactivity Disorder (ADHD)

borderline personality disorder

In the Western World, many women presently use soy products for mood swings in peri- and menopause, when estradiol is fluctuating[10]. In this present invention it is claimed for the first time ever that a special fermented soy formulation is particularly effective in severe mental disturbances like schizophrenia, bipolar disorders, anxiety disorders, borderline personality disorder, etc., basically what may be coined as disturbances of the HPA axis. Besides improvement of the mental disease by a special fermented soy formulation, the soy formulation is equally effective in improving simultaneously accompanied immunity disturbances, infection, and an improvement in metabolic syndrome, which are causing a 25 to 30 year reduction in the life span.

In patients with severe mental illness, second generation antipsychotics or atypical antipsychotics have provided treatment for many patients and families struggling with schizophrenia and bipolar disorder. Despite these advances in treatment, metabolic abnormalities, specifically the metabolic syndrome, are occurring at a greater incidence in both persons with immunity disturbances and persons with severe mental illness. Furthermore, patients with severe mental illness have a high risk developing viral and infections, reducing quality of life or even death and succumb to the disease. Current medication in the field of severe mental diseases do not reflect that mental disease is a disease with many risk factors, frequently they worsen the accompanying metabolic syndrome or infection risk: E.g. antipsychotic drugs, which are effective in schizophrenia and bipolar disorders, worsen blood lipids and raise substantial concern among physicians[6]. Other adverse effects of antidepressants, which are frequently taken with tamoxifen by women after surgery for breast cancer to reduce cancer recurrence, increase breast cancer risk and are ineffective against severe mental diseases[11]. Also, they are not related to show any improvement in auto-immunity. Antidepressant drugs are not effective improving severe mental diseases like schizophrenia, bipolar diseases, etc.

It is now more and more recognized that persons suffering from severe mental disease also suffer from diseases, which reduce life expectancy as well as quality of life[12]. Person, who relocated after Hurricane Katrina outside New Orleans, showed besides sign of stress and anxiety disorders signs of heart disease, increased infections and inflammatory diseases, besides signs of a severe depression [13]. There are now several consensus meetings and statements that psychiatrist should focus on improvement of life expectancy[14].

Psychiatric diseases and disorders (also referred to as mental illnesses or disorders) are described in resources such as the American Psychiatric Association's Diagnostic and Statistical Manual of Mental Disorders, or DSM-IV. Broad categories of mental disorders include, but are not limited to, mood disorders, anxiety disorders, schizophrenia and other psychotic disorders, substance-related disorders, sleep disorders, somatoform disorders, and eating disorders. Examples of mood disorders include bipolar and depression. Other conditions falling within the broader category of disorders described above can be found in the DSM-IV, which is incorporated by reference in its entirety. These are debilitating illnesses that affect millions of people and involve astronomical costs, in terms of treatment, lost productivity, and emotional toll. In 2001, the National Institute of Mental Health published a summary of statistics describing the prevalence of mental disorders in America. In the report, it estimated that 22.1% of Americans ages 18 and older suffer from a diagnosable mental disorder in a given year [15].

When applied to the 2008 U.S. Census, the number of people affected was 44.3 million. Depressive disorders can encompass, among others illnesses, major depressive disorder, dysthymic disorder and bipolar disorder. About 9 to 9.5 percent of the U.S. population ages 18 and older have a depressive condition. It has been reported that the direct cost of depressive disorders is about $80 billion, with two-thirds of it being borne by businesses. The indirect costs associated with depressive disorders, such as lost productivity, are harder to calculate because of events described as people at work but limited in their ability to produce or participate [16]. Another psychiatric condition is anxiety disorders. These disorders can include panic disorder, obsessive-compulsive disorder, post-traumatic stress disorder generalized anxiety disorder, and phobias. Approximately 19.1 million American adults, ages 18 to 54 (about 13.3% of people in this age group in a given year), have an anxiety disorder.

Another common psychiatric condition is eating disorders. There are three main types, anorexia nervosa, bulimia nervosa, and binge-eating disorders. These are psychiatric conditions are often linked to perceived notions about body image and are usually independent of actual body weight or body mass index. The mortality of people with anorexia has been estimated at 0.56 percent per year, or approximately 5.6 percent per decade, which is about 12 times higher than the annual death rate due to all causes of death among females ages 15-24 in the general population[17]. It should be noted that psychiatric illnesses usually present with elements of other psychiatric disorders.

Another psychiatric condition is schizophrenia. In a given year, over 2 million people are clinically diagnosed with schizophrenia, and there is a lifetime prevalence of this disease in approximately 1% of the U.S. population. Schizophrenia is a chronic, debilitating disease that leaves an estimated 75% of treated patients without ever achieving complete recovery. Treatment of schizophrenia with the newer (atypical) antipsychotic medications frequently comes with the side effect of weight gain and possibly diabetes[6].

Exemplary types of schizophrenia include paranoid schizophrenia. These persons are very suspicious of others and often have grand schemes of persecution at the root of their behavior. Hallucinations, and more frequently delusions, are a prominent and common part of the illness. Persons with disorganized schizophrenia (Hebephrenic Schizophrenia) are verbally incoherent and may have moods and emotions that are not appropriate to the situation. Hallucinations are not usually present with disorganized schizophrenia. Catatonic schizophrenia is where a person is extremely withdrawn, negative and isolated, and has marked psychomotor disturbances. Residual schizophrenia is where a person is not currently suffering from delusions, hallucinations, or disorganized speech and behavior, but lacks motivation and interest in day-to-day living. Schizoaffective disorder is where a person has symptoms of schizophrenia as well as mood disorder such as major depression, bipolar mania, or mixed mania. Undifferentiated schizophrenia is where conditions meet the general diagnostic criteria for schizophrenia but do not conform to any of the above subtypes, or there are features of more than one of the subtypes without a clear predominance of a particular set of diagnostic characteristics. Psychiatric diseases and disorders can be found in any age group. Accordingly, these disorders can be found in young adults and adults (defined herein as those aged 65 or under) as well as infants, children, adolescents, and the elderly (defined herein as over the age of 65). In fact, certain segments of the population may be particularly prone to having a condition, such as eating disorders in adolescents and young adults. The elderly may be particularly susceptible to conditions such as depression.

ADHD, one of the most common mental disorders in children and adolescents, also affects an estimated 4.1 percent of adults, ages 18-44, in a given year. ADHD usually becomes evident in preschool or early elementary years. The median age of onset of ADHD is seven years, although the disorder can persist into adolescence and occasionally into adulthood.

Current treatments include psychosocial and behavioral therapy, electroconvulsive therapy, and/or medication. A common form of treatment for psychiatric illnesses, or at least a component of the treatment, is the administration of medication. Needed in the art are molecules that (1) effectively treat those patients resistant to the current antidepressants (e.g., tricyclics, monoamine oxidase inhibitors, selective serotonin reuptake inhibitors) (2) effectively treat depression, anxiety, schizophrenia, or other psychiatric diseases or disorders without the unwanted side effects of the current pharmaceuticals, (3) have a faster onset of therapeutic action, and/or (4) improve physical co-morbidities (e.g., diabetes, pain, weight gain) that often present with and make more difficult the treatment of psychiatric illnesses, such as depression, anxiety, and schizophrenia to name just a few.

It has been known for a long time, that in severe mental diseases pregnenolone and DHEA-S are increased, whereas sexual hormones like Testosterone and estradiol are decreased[18]. DHEA-S plasma levels are directly related to severity and accompanying signs of aggression of schizophrenia[19]. It is the basis of this patent that isoflavone from natural, synthetic or metabolic sources alone or in combination with plant extractions decreases sexual hormones metabolized close to cholesterol, like DHEA and pregnenolone, and increases steroidal hormones at the end of the hormone synthesis like cortisol, estradiol, testosterone and particularly “the androstenes” (androstenediol and androstanediol). The later ones are decreased in severe mental diseases. Only recently [20] it has been recognized that andostenes are extremely important to regulate peripheral as well as cellular immunity, because the magnitude of immunity has been

| Hormone | DHEA:Androstenes:Androstenetriol |
|---|---|
| Magnitude of immunity: | 1:100:1000 |

Although fermented soy is composed of compounds like isoflavones, saponins, etc. which have a direct effect to improve immunity, the basis finding is that they improve especially steroidal compounds, which improve immunity and reduce anxiety. Therefore it may be concluded that the beneficial effects are not obtained by drug delivery, but merely by improving impaired steroidal metabolism. The auto-immunity is improved, however, not by external drug delivery. Isoflavones, either of natural, synthetic or metabolic origin regulate alone or in combination with other plant derived compounds dysbalanced hormone synthesis, so that hormones at the end of synthesis are increased and those at the upper part of hormone synthesis are decreased. The influence of cortisol and estradiol on wellbeing can be found in textbooks of menopause and psychiatry. The increase of “androstenes” by fermented soy balances Th1/Th2 cytokines so that immunity and inflammation is corrected, which are accompanying mental diseases. A decrease of androstenes is correlated to anxiety[21].

Besides increase of wellbeing, isoflavones of natural or synthetic origin alone or in combination with plant extracts application in severe mental diseases corrects unfavorable blood lipids and a pro-diabetic blood profile, which will increase life expectancy. It is known that the pro-metabolic blood profile of patients suffering from severe mental diseases reduces their life expectancy from 25 to 30 years Many autoimmune disorders have a wide clinical spectrum, extending from organ involvement (autoimmune exocrinopathy) to systemic disease and B cell lymphoma. In many autoimmune disorders neuro-endocrine systems are impaired. First, the HPA axis appears to be disturbed, since significantly lower basal ACTH and cortisol levels were found in patients with autoimmune diseases and were associated with a blunted pituitary and adrenal response to ovine corticotropin-releasing factor compared to normal controls. Second, HPA-axis is also involved, since lack of estrogens is associated with human disease and the development of autoimmune exocrinopathy in several experimental models. Finally, exocrine glands are enriched with neuroendocrine-related molecules, adjacent to local autoimmune lesions. Certain clinical manifestations of autoimmune disease like easy fatigue, fibromyalgia and psychological disturbances can be very well explained by mechanisms directly related to disturbances of the neuro-endocrine axis. On the other hand, the molecular and biochemical effects of the inflammatory molecules or cell-to-cell interaction, observed during the local or systemic autoimmune injury with cells and mediators of the neuro-endocrine system, are largely unexplored. It was surprisingly found that fermented isoflavone product from soy, fermented soy, red clover, alfalfa, and other sources, either by natural or synthetic origin, showed tremendous improvement of immunity, fibromyalgia, lupus erythematodes, burnout and many other autoimmune disorders.

It has been found for the first time ever, and what is a claim of this invention, that special soy formulations, fermented soy, red clover, alfalfa, and other sources, either by natural or synthetic origin, can be used to improve severe mental disease like schizophrenia and bipolar disorders. It has been shown in the literature that special soy formulations, fermented soy, red clover, alfalfa, and other sources, either by natural or synthetic formulations can be used for peanut allergy in children[22]. It has also been shown that soy may reduce infections [23]. It is shown here for the first time ever that fermented soy formulation can be used to reduce severe mental diseases as well instantaneously immune disturbances, particularly if combined with severe mental diseases.

In one general aspect, methods provided include the use of special isoflavone containing soy formulations, fermented soy, red clover, alfalfa, and other sources, either by natural or synthetic origin in therapeutically effective amounts for the treatment of a psychiatric disorder, immunity disorders, particularly autoimmune disorders, or a mixture of both in doses comparable to baby soy nutrition formula, compared on body weight. In certain embodiments, the psychiatric disorder is a mood disorder, an anxiety disorder, schizophrenia or other psychotic disorder, a substance-related disorder, a sleep disorder, a somatoform disorder, and/or an eating disorder. In certain embodiments, the psychiatric disorder is bipolar disorder. In certain embodiments, the psychiatric disorder is an obsessive-compulsive disorder. In certain embodiments, methods provided may not include treating an eating disorder. In other embodiments, methods provided may not include treating anorexia. In certain embodiments, methods provided may not include a somatoform disorder. In certain embodiments, special isoflavone containing soy formulations, fermented soy, red clover, alfalfa, and other isoflavone sources, either by natural or synthetic origin is used to treat the underlying psychiatric condition of an eating disorder. In certain embodiments, fermented soy is used to treat the underlying psychiatric condition of a somatoform disorder.

In still other embodiments, the subject has a metabolic condition. In yet other embodiments, the subject has diabetes, metabolic syndrome, impaired glucose tolerance, or insulin resistance and suffers from a severe mental disease.

In another general aspect, methods provided herein include administration of a therapeutically effective amount of fermented soy, alone or in combination with a conventional treatment for psychiatric disorders. In other embodiments, the combination includes the administration of another psychiatric medication together with fermented soy. In still other embodiments, the psychiatric medication is one or more of tricyclic antidepressants, monoamine oxidase inhibitors (MAOIs), selective serotonin reuptake inhibitors (SSRIs), serotonin and noradrenaline reuptake inhibitors (SNRIs), herbal antidepressants (e.g., St John's Wort or Hypericum), or second generation antipsychotic medications.

In another general aspect, methods provided herein include treating an unwanted side effect of another psychiatric medication comprising administering a therapeutically effective amount of fermented soy, to a subject in need thereof. In certain embodiments, the other psychiatric medication is a second-generation antipsychotic medication. In certain embodiments, the unwanted side effect of the other psychiatric medication is weight gain. In other embodiments, the unwanted side effect of the other psychiatric medication is diabetes.

In another general aspect, methods provided include treating a psychiatric disorder comprising administering a therapeutically effective amount of a compound that modulates behavioral pathways through its modulator actions on metabolic pathways or function, including but not limited to hormone metabolism, lipid metabolism, protein metabolism, and total energy metabolism. In certain embodiments, the behavioral pathway is a corticotropin-releasing factor (CRF) pathway that modulates at least one component of the hypothalamic-pituitary-adrenal axis. In other embodiments, the behavioral pathway is the Th1 or Th2 cytokine. In certain embodiments, the metabolic or behavioral pathway is any one of glucoregulatory, glucocorticoid responsive, or stress responsive. In certain embodiments, the compound is fermented soy, isoflavone preparation either of natural or synthetic or metabolic origin, alone or in cooperation with plant products altering NF-kB.

In another aspect, the disclosure provides for the use of special isoflavone containing soy formulations, fermented soy, red clover, alfalfa, and other isoflavone sources, either by natural or synthetic origin for manufacture of a medicament useful for treating psychiatric diseases and disorders described herein. In another aspect, the disclosure provides for the use special isoflavone containing soy formulations, fermented soy, red clover, alfalfa, and other isoflavone sources, either by natural or synthetic origin for manufacture of a medicament useful for treating unwanted side effects of another psychiatric medication, for example, a second generation antipsychotic medication.

In another aspect, the disclosure provides for the use of special isoflavone containing soy formulations, fermented soy, red clover, alfalfa, and other isoflavone sources, either by natural or synthetic origin for treating psychiatric diseases and disorders described herein.

In another aspect, the disclosure provides for the use of special isoflavone containing soy formulations, fermented soy, red clover, alfalfa, and other isoflavone sources, either by natural or synthetic origin for treating psychiatric diseases and accompanying immunological disorders described herein.

In another aspect, the disclosure provides for the use of fermented soy for treating psychiatric diseases and accompanying blood lipid disorders leading to diabetes or metabolic disease described herein

### Stress: Principal Cause for Severe Mental Diseases

There is convincing evidence that environmental stress plays a significant role in modifying both mental and physical health. Biological risks of developing severe mental diseases, following or accompanying mental stress, have been described as well: They can be inherited from their parents, what hints to a genetic influence. Stress during pregnancy can increase the risk of a child to develop severe mental diseases in later life. Increased gene modifications have been described in individuals suffering from severe mental diseases. Although genes modifications are believed to play a role in the development of severe mental diseases, their role has been limited to a sub fraction of patients, which indicates that other factors may be involved. As will be shown in this patent, the governing molecular basis of all severe mental diseases is the disturbances of the HPA-axis and although this has been recognized for many centuries and is textbook knowledge in the medical field, it has not been possible to correct the HPA axis completely. 1) SSRI's are limited to restore Serotonin imbalances and to depression, whereas 2) antipsychotics for Schizophrenia, bipolar disease are not applicable to PTSD and prolactin even more, which endangers partner relationships through reduction of sex drive and increase unfavorable blood lipids reduces life expectancy even more.

It has long been recognized that severe mental problems are related to imbalances of the HPA axis (hypothalamus-pituitary-adrenal-axis), which recognized that adrenals and their hormones are directly involved in the disease. It has been recognized that stress increases DHEA and pregnenolone, two hormones in the upper part of the hormone synthesis. For example in NATO-Elite soldiers conducting winter training in north Norway, these hormones were significantly increased and testosterone, however was decreased. No change in cortisol levels were found, indicating that the soldiers were suffering from physical stress and not from mental stress, because cortisol is a key hormone for depression and severe mental diseases. It may be concluded that severe mental diseases show further alteration of the hormone cascade.

There is no established correction of increases in DHEA and pregnenolone levels, which cause stress in mentally handicapped people and may be the major driving force to develop severe mental diseases.

Correction of similar key physiologic disbalances by a fermented soy product can correct hormone disbalances of the HPA axis and also correct hematology disturbances as well. The key finding of this patent is that they relate to mental disturbances during pregnancy: It has been recognized that postnatal depression is really postnatal stress, and resembles similarities to post-traumatic-stress-disorder (PTSD). This may very well explain that women suffer from anxiety attacks and are hostile to their newborns. The similarity of hormone disbalance during pregnancy explains why women suffer more from schizophrenia or Bipolar disease during pregnancy or in a hospital situation even more. It is a finding that fermented soy can correct hormone disbalance during pregnancy and fermented soy can be taken as a protective treatment for female soldiers during pregnancy or surgery to decrease the risk of developing schizophrenia.

As is depicted in Table 1 and 2, fermented soy corrects these physiologic features, common to almost all severe mental disorders simultaneously, and is therefore suited to correct also other accompanying diseases like immune disorders like viral infection, and metabolic disorders.

It is a key finding of our invention that severe mental disorders are often accompanied by severe treatment resistant diseases normally found in Internal medicine, like treatment resistant hypertension, treatment resistant edema (people with schizophrenia may literally drown in their water in the lung), hematological disorders like increased White Blood Cell (CD4 cells like in leukemia). Since all these severe physiologic currently untreatable diseases have to be controlled, fermented soy may also be taken to treat treatment resistant hypertension, edema, or be taken in combination.

TABLE 1
PHSIOLOGIC COMPLICATIONS IN MENTAL DISEASES and in treatment resistant diseases where hormone
disbalance may be involved (like androstenediol increase in hypertension or edema)

| | | Th1/Th2 | | | | | | | | |
|---|---|---|---|---|---|---|---|---|---|---|
| | | Cytokine | | | | | | Hypertension | | |
| | | Dysbalance | | Low | | | Immune | through | | |
| | | in Blood | DHEA and | cortisol = | | Low | Disturbances | Edema (LOW | Blood | |
| | WBC | Particulalry | Pregnenolone | HPA | Low | Estra- | (e.g. | Aldosteron | Lipids | Diabetes |
| | Increased | IL-6, TNF-alpha | increased | Marker | T | diol | CD4 down) | up) | INCREASED | Risk |
| Physical | — | — | ✓ | ✓ | ✓ | — | — | — | — | — |
| Stress | | | | | | | | | | |
| Treatment | — | — | — | — | — | — | — | ✓ | — | — |
| Resistant | | | | | | | | | | |
| Hypertesion | | | | | | | | | | |
| COPD | — | — | ✓ | — | — | — | ✓✓ | ✓✓ | — | — |
| LUPUS | ✓ | ✓✓ | ✓✓ | ✓✓ | — | — | ✓✓✓ | ✓ | | |
| Schizophrenia | ✓✓ | ✓✓ | ✓✓ | ✓ | ✓✓ | ✓ | ✓✓ | ✓✓ | ✓✓✓ | ✓✓ |
| Bipolar | ✓ | ✓✓ | ✓✓ | ✓✓ | ✓✓ | ✓ | ✓✓ | ✓✓ | ✓✓✓ | ✓✓ |
| Disorder | | | | | | | | | | |
| PTSD | ✓ | ✓✓ | ✓✓ | ✓✓ | ✓✓ | ✓ | ✓✓ | ✓✓ | ✓✓✓ | ✓✓ |
| Postnatal | ? | ✓✓ | ✓✓ | ✓✓ | ? | ? | ✓ | ✓ | ✓ | ✓ |
| Depression = | | | | | | | | | | |
| Postnatal | | | | | | | | | | |
| Stress | | | | | | | | | | |
| Compulsive | ? | ✓✓ | ✓✓ | ✓✓ | | — | — | ✓ | — | — |
| Behavior | | | | | | | | | | |
| Depression | — | — | — | ✓✓ | — | — | (✓) | (?) | (?) | (?) |
| | | | | | | | negligible | | | |
| Tourette | — | — | ✓✓✓ | ✓✓ | — | — | — | — | — | — |
| Syndrome | | | | | | | | | | |
| ADHD | — | — | (✓) | ✓✓ | ? | ? | — | — | — | — |

T means testosterone,

TABLE 2
Influence of fermented soy product on physiologic symptoms defined in Table 1

| | | Th1/Th2 | | | | | | | | |
|---|---|---|---|---|---|---|---|---|---|---|
| | | Cytokine | | | | | | Hypertension | | |
| | | Dysbalance | | Low | | | Immune | through | | |
| | | in Blood | DHEA and | cortisol = | | Low | Disturbances | Edema | | |
| | WBC | Particulalry | Pregnenolone | HPA | Low | Estra- | (e.g. | (Aldosteron | Blood | Diabetes |
| | Increased | IL-6, TNF-alpha | increased | Marker | T | diol | CD4 down) | up) | Lipids | Risk |
| Physical | — | — | — | — | — | — | — | — | — | — |
| Stress | | | | | | | | | | |
| Mental Stress | — | — | — | — | — | — | — | — | — | — |
| Treatment | — | — | — | — | — | — | — | — | — | — |
| Resistant | | | | | | | | | | |
| Hypertesion | | | | | | | | | | |
| COPD | — | — | — | — | — | — | — | — | — | — |
| LUPUS | — | — | — | — | — | — | — | — | — | — |
| Schizophrenia | — | — | — | — | — | — | — | — | — | — |
| Bipolar | — | — | — | — | — | — | — | — | — | — |
| Disorder | | | | | | | | | | |
| PTSD | — | — | — | — | — | — | — | — | — | — |
| Postnatal | — | — | — | — | — | — | — | — | — | — |
| Depression = | | | | | | | | | | |
| Postnatal | | | | | | | | | | |
| Stress | | | | | | | | | | |
| Compulsive | — | — | — | — | — | — | — | — | — | — |
| Behavior | | | | | | | | | | |
| Depression | — | — | — | — | — | — | — | — | — | — |
| Tourette | — | — | — | — | — | — | — | — | — | — |
| Syndrome | | | | | | | | | | |
| ADHD | — | — | — | — | — | — | — | — | — | — |

### Further Background

Hormones play a dominant role in the etiology of mood swings and disorders besides severe mental diseases, for example during the female hormone cycle, or in menopause, or even in winter depression living in Nordic countries. The decline of female ovarian hormone plays a significant role in menopause. Their substitution by oral, transdermal or via injectable delivery may serve as a textbook example, proving that hormones are involved in mood disorders. Normally, the female hormone estradiol is known to be responsible for correcting mood swings and physical conditions in menopause, like hot flushes, dry vagina and poor skin. Although estradiol and its pre-cursers or metabolites are known as a means to correct mood disorders in menopause as well, it may not be generally known that for many menopausal women injectable delivery, avoiding the first pass metabolism of the liver, of the adrenal hormone DHEA improves wellbeing dramatically as well. DHEA is partially metabolized to estradiol, so it is assumed that DHEA facilitates its effect in menopausal women via estradiol. However, the strong androgenic effect improving assertiveness, sexuality, and alertness, documents that other effects besides estrogenic effects may be involved. Many leading Gynecologists therefore question “mono-therapy”, the use of one hormone, because many sexual hormones may be needed. Unfortunately only men and women with financial resources are able to correct several hormones in aging medicine to improve mental and biological conditions, which has not been adopted by classical medicine and is left to trained specialist and those skilled in the art.

Severe mental diseases are fundamentally different from mood disorders in menopause, where decline of ovarian hormones are responsible. The evidence suggests people with schizophrenia, Bipolar Disorders, and post-traumatic stress disorders, postnatal depression can experience both hyper- and hypo-function of the HPA axis (hypothalamic-pituitary-adrenal (HPA) axis). It is likely, and this is the basis finding of this patent that this contributes to the pattern of poor physical health and premature mortality, as well, particularly high rates of cardiovascular and metabolic disturbance. Also viral diseases are common in patients suffering from severe mental diseases and what is not commonly known, changes in blood hematology (Table 1). It is the key claim of this patent that a correction of HPA-Axis with fermented soy will not only correct mental disturbances but also cardiovascular and metabolic disturbances, and accompanying immune disturbances (Table 2). The effect to improve mental and biological health in patients suffering from severe mental disturbances by fermented soy is clearly concentration dependent and requires fermented soy in concentrations compared to soy baby nutrition formula, that means normally higher than 300 mg isoflavones per day, depending on patient weight, where a dose preferably between 500 to 1200 mg soy isoflavones is recommended.

Unfortunately, second-generation antipsychotics, which are currently used in the treatment of all forms of psychoses and are currently the method of choice, often produce undesirable side effects, among them weight gain and other elements of metabolic syndrome. This is detrimental to patient health, since they are suffering from poor blood lipids and metabolic disorders already, which may impair life expectancy even more: As was demonstrated in epidemiologic studies severe mental diseases like schizophrenia, Bipolar Disorders, do reduce life expectancy between 25-30 years. The mechanisms of adverse effects of first and second generation anti-psychotics are not known, but increased triglycerides may be the most severe negative impact increasing infarction and stroke risk of these patients even more.

Contributing evidence suggests that increased levels of prolactin (hyper-prolactinemia) associated with some antipsychotics raises the risk of sexual side effects and impairs the partner relationships even more. Scientists criticize that anti-psychotics cause a numbness without actually improving the disease, although it cannot be overlooked that antipsychotics improve some physiologic functions, like reductions of pathologic White Blood Counts and increased potassium values, just to name a few, they are accompanied with severe side effects. From these few examples it can be concluded that current treatment of severe mental disease is suboptimal in terms of clinical and physiologic needs, because first and second generation anti-psychotics have no effect on severe immunity disturbances. Since these medications provide no cure, just an improvement in some selected symptoms an improved treatment for all features is highly needed. For example 25% of all schizophrenia patients suffer from severe life threatening edema because they drown in their own water accumulating in the lungs. Since no medical established treatment for this disease has been established for normal patients, it is a surprising finding of this patent that it prevents edema, although it has been known that COPD is reduced by soy.

It is discussed that severe mental diseases may reduce compliance to adhere to therapy, as well increase suicide and this may be a major risk to reduce life expectancy. Although this may be contributing factors, it is now accepted that metabolic diseases, increased lung diseases, and viral infections are major factors in reducing life expectancy. Therefore an improved treatment, correcting mental and physiologic disease together is strongly needed.

Key severe mental disturbances, which are mediated by HPA axis disturbances, are: A) “bipolar disorder” is of fairly recent origin and refers to the cycling between high and low episodes (poles). A relationship between mania and melancholia had long been observed, although the basis of the current conceptualization can be traced back to French psychiatrists in the 1850s. The term “manic-depressive illness” or psychosis was coined by German psychiatrist Emil Kraepelin in the late nineteenth century. Data from the United States on lifetime prevalence varies; but it indicates a rate of around 1% for bipolar I, 0.5%-1% for bipolar II or cyclothymia, and 2%-5% for sub-threshold cases meeting some, but not all, criteria.

B) Schizophrenia is a mental disorder characterized by a disintegration of the process of thinking and of emotional responsiveness.[1] It most commonly manifests as auditory hallucinations, paranoid or bizarre delusions, or disorganized speech and thinking, and it is accompanied by significant social or occupational dysfunction. The onset of symptoms typically occurs in young adulthood,[2] with a global lifetime prevalence of around 0.3-0.7%.[3] Diagnosis is based on the patient's self-reported experiences and observed behavior.

c) Obsessive—compulsive disorder (OCD) is an anxiety disorder characterized by intrusive thoughts that produce uneasiness, apprehension, fear, or worry, by repetitive behaviors aimed at reducing anxiety, or by a combination of such thoughts (obsessions) and behaviors (compulsions). Symptoms may include repetitive hand washing; extensive hoarding; preoccupation with sexual or aggressive impulses, or with particular religious beliefs; aversion to odd numbers; and nervous habits, such as opening a door and closing it a certain number of times before one enters or leaves a room. These symptoms can be alienating and time-consuming, and often cause severe emotional and financial distress. The acts of those who have OCD may appear paranoid and come across to others as psychotic. However, OCD sufferers generally recognize their thoughts and subsequent actions as irrational, and they may become further distressed by this realization.

OCD is the fourth-most-common mental disorder, and is diagnosed nearly as often as asthma and diabetes mellitus. In the United States, one in 50 adults have OCD. Obsessive Compulsive Disorder affects children and adolescents as well as adults. Roughly one third to one half of adults with OCD report a childhood onset of the disorder, suggesting the continuum of anxiety disorders across the life span.

d) Postpartum depression also called postnatal depression, is a form of clinical depression, which can affect women, and less frequently men, after childbirth. Studies report prevalence rates among women from 5% to 25%, but methodological differences among the studies make the actual prevalence rate unclear. Among men, in particular new fathers, the incidence of postpartum depression has been estimated to be between 1.2% and 25.5%.[1] Postpartum depression occurs in women after they have carried a child, usually in the first few months, and may last up to several months or even a year. Symptoms include sadness, fatigue, and change in sleeping and eating patterns, reduced libido, crying episodes, anxiety, and irritability. It is sometimes assumed that postpartum depression is caused by a lack of vitamins, but studies tend to show that more likely causes are the significant changes in a woman's hormones during pregnancy. On the other hand, classical hormonal treatment has not helped postpartum depression victims. It is now more and more recognized that postnatal depression is in reality postnatal stress, because hormone changes are similar to post-traumatic stress syndrome, explain why some mothers are hostile to their newborn babies.

### Attention Deficit Disorders

e) Attention deficit hyperactivity disorder (ADHD or AD/HD or ADD) is a neurobehavioral[1] developmental disorder. It is primarily characterized by “the co-existence of attention problems and hyperactivity, with each behavior occurring infrequently alone” and symptoms starting before seven years of age.

ADHD is the most commonly studied and diagnosed psychiatric disorder in children, affecting about 3 to 5 percent of children globally and diagnosed in about 2 to 16 percent of school aged children. It is a chronic disorder with 30 to 50 percent of those individuals diagnosed in childhood continuing to have symptoms into adulthood. Adolescents and adults with ADHD tend to develop coping mechanisms to compensate for some or all of their impairments. It is estimated that 4.7 percent of American adults are estimated to live with ADHD.

ADHD is diagnosed two to four times as frequently in boys as in girls, though studies suggest this discrepancy may be partially due to subjective bias of referring teachers. ADHD management usually involves some combination of medications, behavior modifications, lifestyle changes, and counseling. Its symptoms can be difficult to differentiate from other disorders, increasing the likelihood that the diagnosis of ADHD will be missed. Additionally, most clinicians have not received formal training in the assessment and treatment of ADHD, particularly in adult patients.

ADHD and its diagnosis and treatment have been considered controversial since the 1970s. The controversies have involved clinicians, teachers, policymakers, parents and the media. Topics include the actuality of the disorder, its causes, and the use of stimulant medications in its treatment. Most healthcare providers accept that ADHD is a genuine disorder with debate in the scientific community centering mainly around how it is diagnosed and treated. The American Medical Association concluded in 1998 that the diagnostic criteria for ADHD are based on extensive research and, if applied appropriately, lead to the diagnosis with high reliability. It is interesting to note, that ADHD starts with seven years of age in the adrenarche, when the adrenal hormone ANDOSTENEDIOL is increased, which is need for developing bone and muscle. Adrenarche is the time in life of a human when the highest growth in height takes place with the increase of the hormone androstenediol. It has to be recognized that androstenediol is an adrenal hormone, a key hormone of the HPA-axis, where A denotes “adrenal”.

f) Systemic lupus erythematosus, Lupus is often abbreviated to SLE or lupus, is a systemic autoimmune disease (or autoimmune connective tissue disease) that can affect any part of the body (Table 1). As occurs in other autoimmune diseases, the immune system attacks the body's cells and tissue, resulting in inflammation and tissue damage. It is a Type III hypersensitivity reaction caused by antibody-immune complex formation. SLE most often harms the heart, joints, skin, lungs, blood vessels, liver, kidneys, and nervous system. The course of the disease is unpredictable, with periods of illness (called flares) alternating with remissions. The disease occurs nine times more often in women than in men, especially in women in child-bearing years ages 15 to 35, and is more common in those also of non-European descent. SLE is treatable through addressing its symptoms, mainly with cyclophosphamide, corticosteroids and immunosuppressant; there is currently no cure. SLE can be fatal, although with recent medical advances, fatalities are becoming increasingly rare. Survival for people with SLE in the United States, Canada, and Europe is approximately 95% at five years, 90% at 10 years, and 78% at 20 years.

g) Tourette syndrome (TS) is an inherited neuropsychiatric disorder with onset in childhood, characterized by multiple physical (motor) tics and at least one vocal (phonic) tic; these tics characteristically wax and wane. Tourette's is defined as part of a spectrum of tic disorders, which includes transient and chronic tics. Subjects with TS and obsessive compulsive behavior (OC) experienced significantly more psychosocial stress than did the controls. Estimates of psychosocial stress were predictive of future depressive symptoms. Current levels of psychosocial stress also area significant predictor of future OC symptom severity, but not vice versa. Current OC symptom severity was a predictor of future depressive symptom severity, but not vice versa. Current levels of psychosocial stress and depression were independent predictors of future tic severity.

### BRIEF DESCRIPTION OF THE DRAWINGS

FIG. 1 shows alterations in the circadian rhythm for testosterone and estradiol in a control experiment, during short and prolonged continuous stress and during recovery of Norwegian Elite soldiers.

FIG. 2 shows the circadian rhythm for cortisol, progesterone, dihydroepiandrosterone sulfate, androstenedione, dihydroepiandrosterone and 17α-hydroxyprogesterone during a control experiment with normal school activities (left column), during the first 24 hours of continuous activities (mid-left) and from 72 to 97 hours of activities (mid-right) during a military training course with continuous physical activities almost without sleep and with limited amounts of food.

FIG. 3 shows cyclic adenosine monophosphate (cAMP) response to adrenaline stimulation in human mononuclear cells and granulocytes during a 5-day military training course with heavy physical activities, sleep and energy deficiency.

FIG. 4 shows the circadian rhythm for mental performance expressed as the mean of two mental performance tests; the code test and the logical reasoning test.

FIG. 5 depicts the normal hormone cascade starting from cholesterol and the sexual hormone cascade is schematically determined.

FIG. 6 is a schematic depiction of impaired hormone synthesis during severe mental disease (schizophrenia, Bipolar Disorders, PTSD, Convulsive disorders).

FIG. 7 shows an increase in hormones found in patients with severe mental disease, and, above the dotted line, incorporates the “stress hormones” DHEA and pregnenolone.

FIG. 8 shows that the fluctuations of Adiol, the metabolite of DHEA during pregnancy, in the first trimester can be related to a decrease of immunity so the blastocyst can grow, however it increases the risk of losing the baby.

FIG. 9 shows that the “Androstenes” have effect on immunity by modifying Th1, Th2 cytokines, which then effect anti-viral and anti-bacterial effects.

FIG. 10 shows the relationship of Androstenediol (Adiol) and cytokines during pregnancy.

FIG. 11 shows the effect of a fermented soy product on the hormone cascade depicted in FIG. 7.

FIG. 12 is a schematic depiction of hormones and their relationship to physiologic functions.

FIG. 13 shows laboratory parameters where the percentage of abnormal values were significantly changed between the acute and recovery phases of Schizophrenia.

FIG. 14 is a schematic depiction of the relationship between androstenediol, and White blood counts and CD4 cells.

FIG. 15 is a funnel plot of results, from included published studies, on the effects of low doses of soy protein and isoflavones (up to 120 mg) on circulating total estradiol (E2, pmol/l) in post-menopausal women (From: Hooper et al, Hum Reprod Update. 2009; 15: 423-440).

FIG. 16 shows the relationship between androstenediol and age in boys (O) and girls (□) (Taken from (Rohr et al Horm Mol Biol Clin Invest 2010; 3(2)).

FIG. 16a shows the HPA marker in fibromyalgia.

FIG. 16b is a schematic depiction of comorbidity of fibromyalgia.

FIG. 17 shows biosynthetic pathways of steroidal hormones.

FIG. 18 shows structure formulas of androstenes and soy isoflavones.

FIG. 19 shows that metabolism of DHEA is inhibited in women suffering from PTSD as a consequence of sexual abuse during childhood.

FIG. 20 is a schematic depiction of the relationship between andostenes and immunity and inflammation.

FIG. 21 shows that there is a complete reduction of stress of 9 NATO-ISAF Soldiers suffering from PTSD, according to the EORTC QLQ-C30 (version 3) questionnaire.

FIG. 22 shows the influence of consumption of fermented soy on anxiety in NATO-ISAF-soldiers suffering from PTSD, according to the EORTC QLQ-C30 (version 3) questionnaire.

FIG. 23 shows the influence of fermented soy on sleeping habits of NATO-ISAF soldiers suffering from PTSD, according the EORTC QLQ-C30 (version 3) questionnaire.

FIG. 24 shows employment of NATO-ISAF soldiers suffering form PTSD after consumption of fermented soy (SOY-IQ), based on a clinical trial.

FIG. 25 shows an improvement of blood compounds related to metabolic syndrome in NATO-ISAF-soldiers suffering from PTSD, by consumption of fermented soy.

FIG. 26 shows the Influence of consumption of fermented soy on DHEA-S blood values in NATO-ISAF-soldiers suffering from PTSD.

FIG. 27 shows a reduction of 17-OH-Pregnenolone in serum of NATO-ISAF soldiers suffering from PTSD under consumption of fermented soy.

FIG. 28 shows an increase of testosterone by fermented soy in PTSD patients.

FIG. 29 shows an individual Increase of body weight in cachectic NATO-ISAF soldiers suffering from PTSD after 3-month consumption of fermented soy.

FIG. 30 shows the effect of consumption of fermented soy on wellbeing in NATO-ISAF soldiers suffering from PTSD, according to the EORTC QLQ-C30 (version 3) questionnaire.

FIG. 31 is a summary of an investigation of hormonal steroids in serum of PTSD patients.

FIG. 32 illustrates that anti-psychotics and fermented soy showed considerable differences in preventing readmission to return to the hospital for treatment of schizophrenic episodes.

FIG. 33 depicts the onset of pharmacologically relevant effect reducing schizophrenia attacks.

FIG. 34 is a comparison of the reduction of average schizophrenic symptoms based on PANSS after 3 months in 3 different arms of treatment.

FIG. 35 shows the effect of schizophrenic treatment on selected blood markers, discussed in schizophrenic patients.

FIG. 36 shows the effect of treatment with various regimens on stress hormone DHEA-S plasma levels after 3-months treatment in schizophrenic patients in the acute phase.

FIG. 37 shows the effect of treatment with various regimens on testosterone plasma levels after 3-months treatment in schizophrenic patients in the acute phase.

FIG. 38 shows the effect of treatment with various regimens on estradiol plasma levels after 3-months treatment in schizophrenic men in the acute phase.

FIG. 39 shows that the onset of labor is dictated by the fetal genome proceeding through either a fetal growth pathway with increases in uterine stretch or fetal endocrine pathway involving activation of the fetal HPA axis.

### DETAILED DESCRIPTION OF THE INVENTION

### Normal Mental and Physical Stress

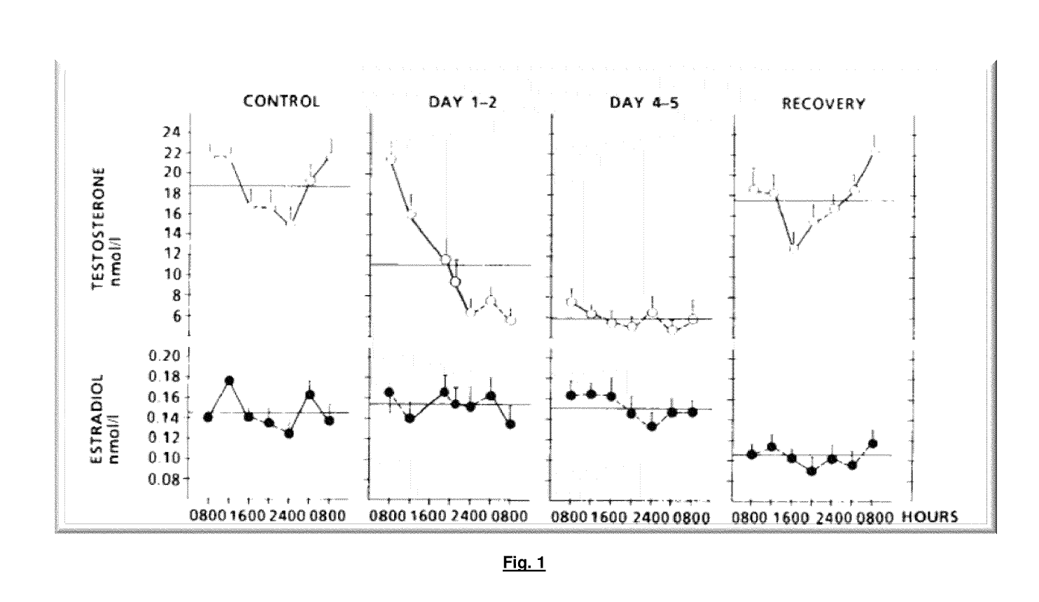

As a textbook example of effect of stress on exhaustion and changes in endocrine hormone levels, a study in Elite NATO-Soldiers is cited, which was conducted in winter in North Norway (FIG. 1,2,3,4).

FIG. 1: Alterations in the circadian rhythm for testosterone and estradiol in a control experiment, during short and prolonged continuous stress and during recovery of Norwegian Elite soldiers. The circadian rhythm during a control experiment with normal school activities (left column), during the first 24 h of continuous activities (mid-left) and from 72 to 97 h of activities (mid-right) during a military training course with continuous physical activities almost without sleep and with limited amounts of food. The recovery experiment (right column) was performed 4-5 days after the course, while the cadets had normal school activities. The blood samples were collected at 4 hour intervals. The results are expressed as means±sem. The time to time variations that were statistically significant at p<0.01 are shown with thick lines and those that were not significant by dotted lines. Horizontal lines indicate 24-h means. (Taken from P. K. Opstad, Endocrine and Metabolic Changes during Exhaustive Multifactorial Military Stress. 2002).

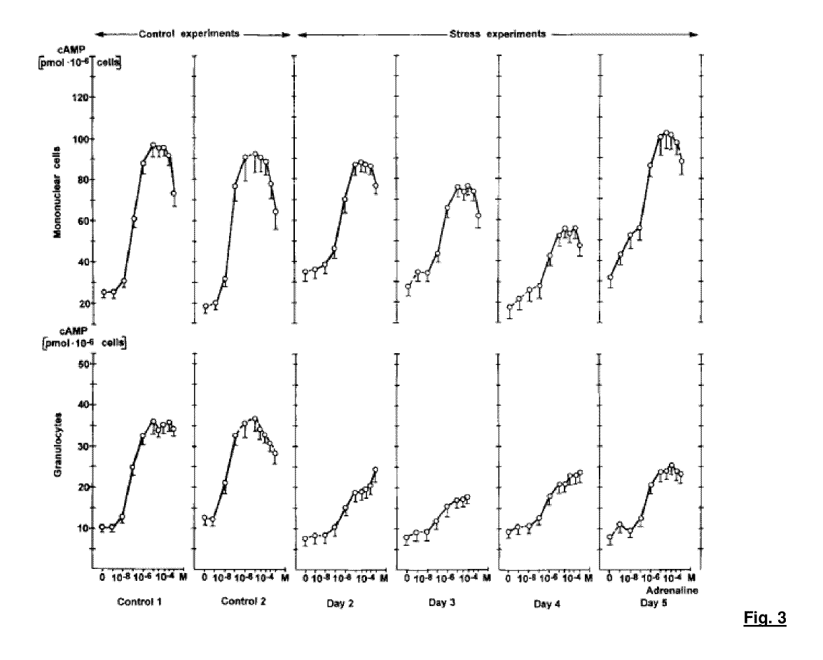

It is now established that continuous physical and emotional stress will lead to dramatic changes in the endocrine synthesis. Some hormones are decreased, like testosterone (FIG. 1), whereas others typical stress hormones increased like DHEA, cortisol (FIG. 3). The later is textbook knowledge that they are stress hormones.

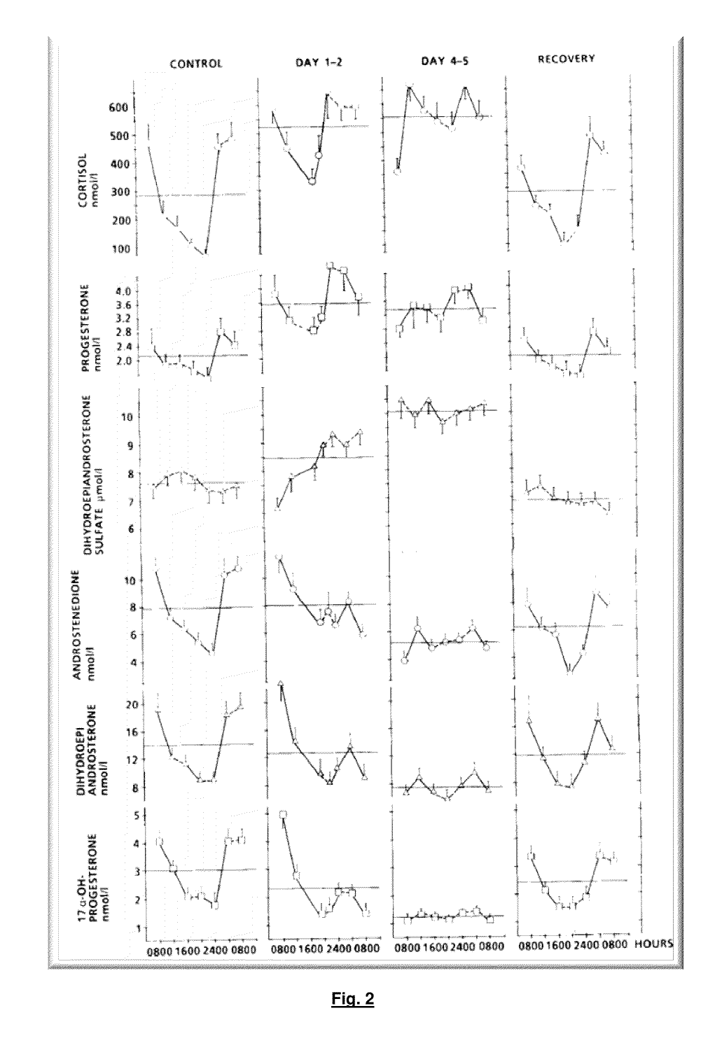

FIG. 2: The circadian rhythm for cortisol, progesterone, dihydroepiandrosterone sulfate, androstenedione, dihydroepiandrosterone and 17α-hydroxyprogesterone during a control experiment with normal school activities (left column), during the first 24 h of continuous activities (mid-left) and from 72 to 97 h of activities (mid-right) during a military training course with continuous physical activities almost without sleep and with limited amounts of food. The recovery experiment (right column) was performed 4-5 days after the course, while the cadets had normal school activities. The blood samples were collected at 4 hrs intervals. The results are expressed as means±sem. The time to time variations that were statistically significant at p<0.01 are shown with thick lines and those that were not significant by dotted lines. Horizontal lines indicate 24-h means. (Taken from P. K. Opstad, Endocrine and Metabolic Changes during Exhaustive Multifactorial Military Stress. 2002).

Something what is known to the expert, is that stress has a dramatic effect on blood hematology (FIG. 3, 4). Immunity is mediated via a whole battery of physiologic functions, but monocytes and granulocytes are considered as part of the immune defense system. It is common knowledge that constant stress can hamper your immune system and you may suffer from severe viral infections and inflammation.

FIG. 3: Cyclic adenosine monophosphate (cAMP) response to adrenaline stimulation in human mononuclear cells and granulocytes during a 5-day military training course with heavy physical activities, sleep and energy deficiency. The experiments were performed on days 2-5 and in two control experiments performed while the cadets had normal activities at the training Academy. The results are shown as means and SEM. Time to time variations statistically significant at P<0.01 are shown with thick lines. (Taken from P. K. Opstad, Endocrine and Metabolic Changes during Exhaustive Multifactorial Military Stress. 2002).

The here depicted endocrine effects and effects on blood hematology can be related to mental performance. The investigation shows rather large endocrine and metabolic alterations during a 5-day military training course with continuous physical activities combined with sleep and energy deficiency, which might contribute to explain the accompanying alterations in both mental and physical performance. The main stress factors such as physical strain, lack of food and sleep all lead to an extreme catabolic metabolism with similarities to the physiological state of the multi-traumatized patients.

During the course there is a general decrease in mental performance; in addition the decrease is stronger during nighttime than during daytime leading to increased amplitude of mental performance.

This is not only due to the darkness of the night but also to endogenous circadian rhythms, since the same alterations are found in the courses organized in June, when the nights are rather bright (Experiments were conducted in Norway where you have summer nights.). All clinical symptoms are also more pronounced at night than at daytime. Almost all illusions, misperceptions, hallucinations, balance disturbances, and coordination problems are worse at night than during the day (see ref Opstad above).

In contrast to the alterations in the circadian rhythm for mental performance, which show increased amplitude during the course, the circadian rhythm of hormones are all extinguished during the course. The circadian rhythms are regulated from the nucleus supra-chiasmaticus in the anterior hypothalamus (Van den Pol and Powley 1979, Rietveld 1992). This center is thought to regulate most of the known circadian rhythms.

Previously it has been shown by Folkard (1985) that the period of the circadian rhythm for the feeling of alertness or drowsiness and deep body temperature dissociated when the period was shortened by 0.2 hours each day down to a “day” of 23 hours and then by 0.1 hour until a “day” of 22 hours which was run for the rest of the study. During the present military training course, it is shown for the first time that during continuous operations, a dissociation may appear between the amplitude of the circadian rhythm for mental performance which is increased, and the amplitude of circadian rhythm for steroid hormones which is extinguished. This indicates that these two rhythms are regulated by different mechanisms in the brain

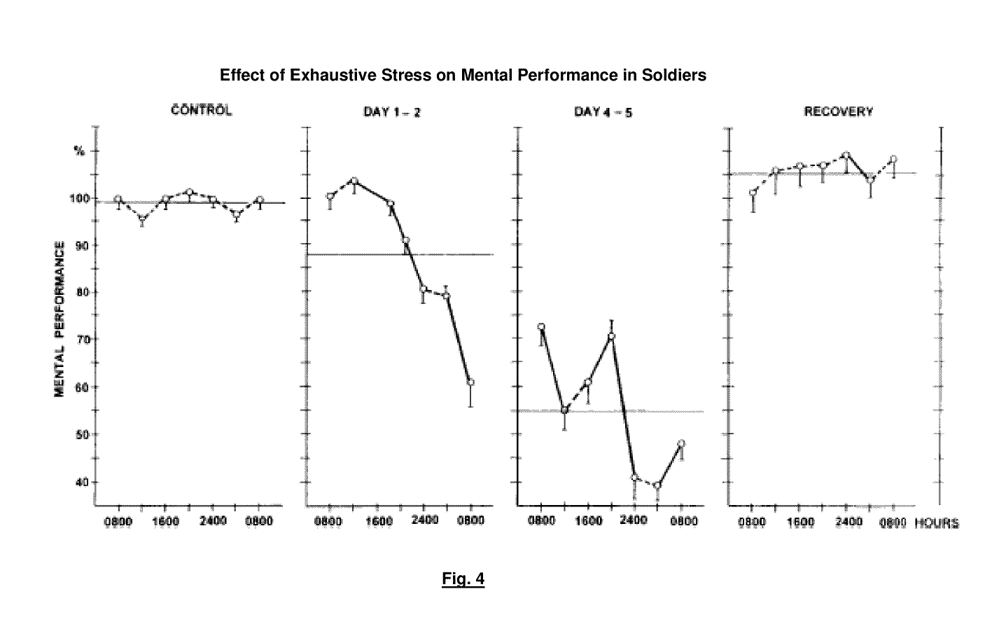

FIG. 4: The circadian rhythm for mental performance expressed as the mean of two mental performance tests; the code test and the logical reasoning test. The results are presented as percent of the first test results (control at 08.00 h) Å} SEM. (Taken from P. K. Opstad, Endocrine and Metabolic Changes during Exhaustive Multifactorial Military Stress. 2002).

### Endocrine Effect in Severe Mental Diseases

FIG. 5 depicts the normal hormone cascade starting from cholesterol. It is a key finding of this patent that almost all hormones altered in stress are also altered in severe mental diseases (SMD). The “Final” hormones of different hormone pathways can be determined as cortisol, aldosterone, and androstanediol (FIG. 6).

FIG. 5: Sexual hormone cascade schematically determined.

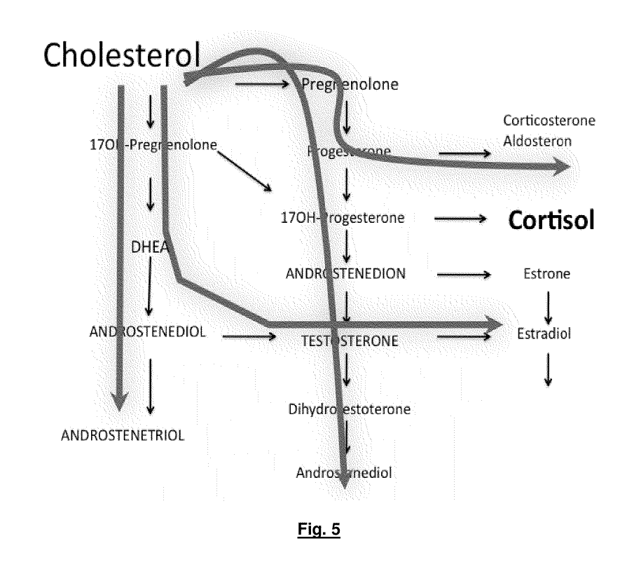

Stress is always part of severe mental disease like, PTSD, Schizophrenia, Bipolar, and postnatal depression, the whole hormone cascade is disturbed: As can be seen in FIG. 7

FIG. 6: Schematic depiction of impaired hormone synthesis during severe mental disease (schizophrenia, Bipolar Disorders, PTSD, Convulsive disorders). SMD denotes for severe mental disease. As can be seen all hormones above the dotted line are increased, hormones lower than the dotted line are decreased.

Hormone metabolism and hormone cascade are severely disturbed, indicating that cholesterol is increased, and all hormones above the dotted line, whereas all hormones below the dotted line are decreased. These conditions are shown to produce detrimental effects on mental and biological health.

Although fractions of the hormone synthesis have been described (for example DHEA increase and Pregnenolone increase and the decrease of cortisol), the total hormone biosynthesis has not been described in total for severe mental disease before.

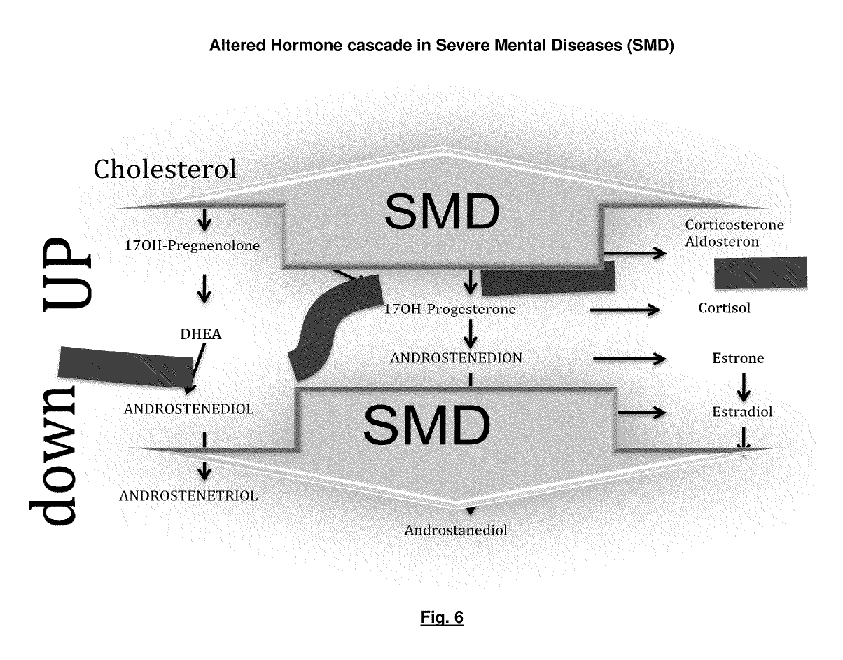

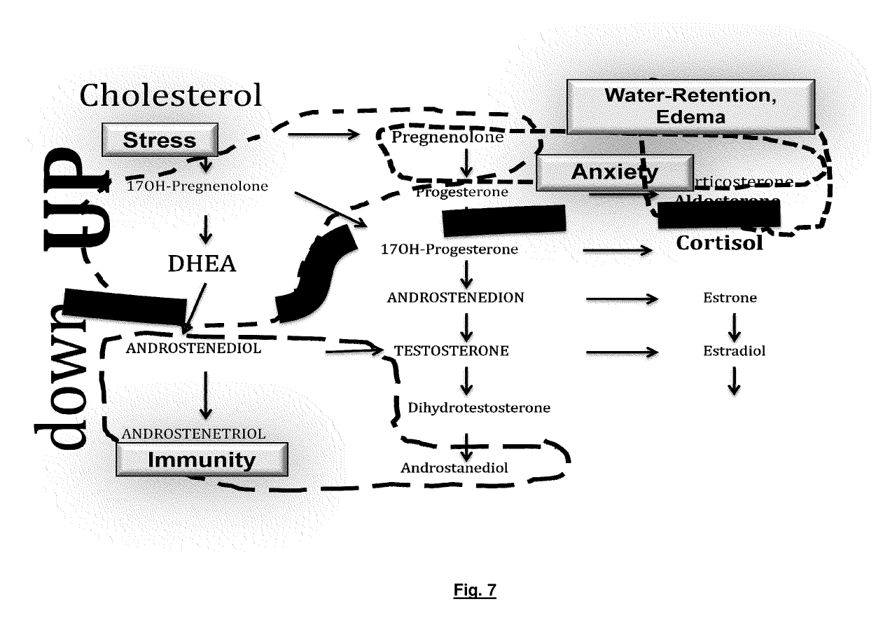

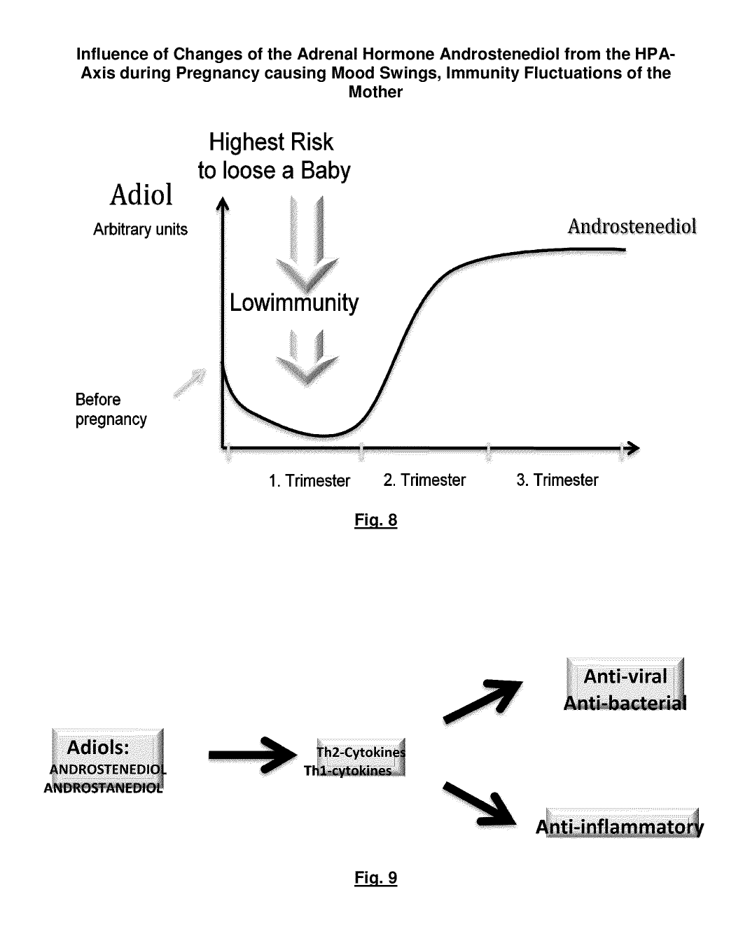

It the key finding of this patent that formulations of fermented soy like isoflavones in concentration like in baby nutritional formula are capable to normalize the whole hormone cascade (FIG. 8)

FIG. 7: Increased hormones found in patients, with severe mental disease, above the dotted line incorporates also the “stress hormones” DHEA and pregnenolone.

It is established that cortisol is decreased in SMD, as well as Estradiol. If however depression accompanies a SMD, then cortisol may increase. In certain situations, a normal cortisol level in schizophrenic patients may indicate therefore that depression is superimposing schizophrenia.

SMD also raises aldosterone, which causes severe edema in the lungs and legs, which has never been described for humans in normal in stress situations. Supporting evidence for our finding is that despite their classification on the DSM scale, Schizophrenia, Bipolar, and Compulsive Disorder, have been reported very similar on the genetic and molecular hormonal level, particularly on the HPA axis. There may be a common denominator with only few exceptions, which are difficult to define. Genes linked to an increased risk of schizophrenia and bipolar disorder as found in recent genome wide association studies appear to be partly separate and partly overlapping between the two disorders.

Although recent investigations have revealed in some patients suffering from schizophrenia or Bipolar disorders that target genes describing the disease in all patients, do not exist. Interestingly, the differences on the genetic levels have been found minimal on the genetic level between Bipolar and Schizophrenia, supporting our finding that correcting sexual hormone cascade will correct many SMD. Gene expression studies have revealed that depression is by no means comparable to patients suffering from SMD.

Genetic linkage studies have produced specific evidence of chromosomal regions increasing susceptibility that Schizophrenia, in a small minority of cases, has been associated with rare deletions or duplications of tiny DNA sequences, occurring within genes involved in neuronal signaling and brain development/human cognitive, behavioral, and psychological variation. Similar relations have been found between autism spectrum disorders and schizophrenia, so that many severe mental diseases share similarities on the genetic level.

SMD share many endocrine properties, relating them to postnatal depression. Since pregnancy is regulated via HPA axis also, it may not come as a surprise that pregnancy depression and postnatal depression are linked to severe mental diseases. It is interesting that pregnancy can trigger latent Schizophrenia, Bipolar, PTSD, which concerns the military veterans army hospitals it female soldiers are treated outside their facilities and physicians are not aware of this fact. The whole pregnancy is regulated via the HPA-axis. Generally, pregnancy is reducing stress and depression, but there are cases that stress, depression, and even schizophrenia, and SMD, is increased during pregnancy. Adrenal hormones of the HPA-axis like androstenediol are altered to reduce the immunity of the mother so that the blastocyst can grow in an explosive manner in the first trimester of the pregnancy. That is the reason why in the beginning of a pregnancy, most women complain about lack of well being, vomiting and sometimes of light memory loss.

FIG. 8: The fluctuations of Adiol, the metabolite of DHEA during pregnancy in the first trimester can be related to a decrease of immunity so the blastocyst can grow, however it increases the risk of losing the baby. Mothers have frequently mood swings in the first trimester.

The birth is a traumatic event for some women, however cannot be explained by increased pain. The fact that birth can be a traumatic event for some mothers, it has to be recognized that the HPA axis is also controlling pregnancy (FIG. 8,9,10,43). After birth the cortisol levels are down in almost all women but it stays low in some women, particularly in women suffering from postnatal depression. Psychiatrists argue that postnatal depression is better named “postnatal stress” and similar to PTSD, explaining that aggression against the babies increases and can have negative effect for the mother and her born child. New findings indicate that postnatal depression is in reality postnatal stress and can be compared to posttraumatic stress disorders. New analysis of endocrine levels of depressed pregnant women has indicated similarity of HPA alterations to stress of post-traumatic stress disorder of soldiers. Generally it is established that a healthy pregnancy reduces stress and depression.

FIG. 9: The “Androstenes” have effect on immunity by modifying Th1, Th2 cytokines, which then effect anti-viral and anti-bacterial effects. Decline of the “androstenes” takes place with age, pregnancy, immunity disturbances and cancer. Androstenediol is metabolized from DHEA. Androstanediol is metabolized from Testosterone. Increases in Androstenediol and Androstanediol improves immunity, their declines decreases immunity.

The androstenes (androstanediol and androstenediol are decreased) to increase the cytokines (inflammatory hormones, particularly TNF-alpha) which will automatically cause rupture of membranes so that birth can take place) (FIG. 11). It is interesting to note that particularly the Th1 cytokines are increased in all patients with SMD, what cause ruptures of capillary membranes which will then cause stroke and heart infarction, a leading cause why patients with SMD are dying 25 to 30 years prematurely. All patient with SMD suffer from extremely poor blood lipids.

FIG. 10: Relationship of Androstenediol (Adiol), cytokines during pregnancy. Increase of cytokines can cause membrane rupture causing birth or miscarriage, but are related to mood swings in the first trimester in pregnancy. There is a relationship between adiol and cytokines (FIG. 9,10). In mental diseases the increase of cytokines may very well increase membrane fracture like stroke and infarction.

For the first time ever it is shown that a fermented soy product can reverse endocrine alterations completely (FIG. 11).

FIG. 11: Effect of a fermented soy product on hormone cascade depicted in FIG. 7. It normalizes cortisol, a marker of the HPA axis and depression and severe mental diseases.

As will be shown, severe mental diseases are composed of several diseases: stress, COPD, treatment resistant hypertension including edema, and immunity reduction like LUPUS as a severe immune disturbance (Table 1). Since these entire diseases shares HPA axis dysbalance, patients are frequently suffering from several mental diseases, and/or exhibit edema, immune disturbances, lung diseases and viral infections. Clearly the cause for this disease is the mental dysbalance, which is then followed, in most cases, by severe untreatable diseases like Lupus, where mental dysbalances are diagnosed in childhood and severe immune disturbances are followed in later years.

FIG. 12: Schematic depiction of hormones and their relationship to physiologic functions. Stress is related to DHEA and pregnenolone. Anxiety disturbances can be related to several hormones like pregnenolone, corticosterone, and aldosterone, edema can be caused by increased aldosterone, which increases sodium and you find water retention. Immunity disturbances can be related to a reduction of the androstenes, and a reduction of cortisol is a marker of severe mental disease. The increased cholesterol is a hint to the disturbed blood lipids, which cause infarction, stroke, causing a reduction of a life span of up to 30 years.

It is the main finding of this patent that soy corrects for hormone dysbalances of the HPA axis (FIG. 12), corrects clinical symptoms of severe mental diseases and accompanying physical disease like LUPUS, treatment resistant hypertension, and edema, metabolic disease. It is the key finding that all these single diseases are treatment resistant. The method of choice for treatment is cortisone, which bring relief to patients suffering from LUPUS. However they cannot be taken for prevention and increase the risk of an infection. Adiol is a superior anti-inflammatory agent compared to cortisones, because it shows strong anti-viral and anti-inflammatory effects (Loria et al. 2010)(FIG. 9).

The relationship between severe immune diseases and mental diseases has been recognized. The American College of Rheumatology has defined 19 neuropsychiatric syndromes associated with systemic lupus erythematosus (SLE) involving the central, peripheral, and autonomic nervous systems. Neuropsychiatric manifestations of lupus (NPSLE) have been shown to occur in up to 95% of pediatric patients with SLE. Given the especially high prevalence of NPSLE in pediatric patients with lupus, it is important for clinicians to recognize that neuropsychiatric symptoms in an adolescent patient may indeed be the initial manifestations of SLE, as opposed to a primary affective disorder. (Alao et al. Psychosomatics 2009; 50:543-547).

### Mental Disorders are HPA-Axis Related

The importance of the HPA axis in controlling mental well-being has been recognized. It is less known, that the importance of the HPA axis to regulate human reproduction has been recognized in Gynecology also. HPA axis allows separating the immunity of the mother and the fetus, enabling the fetus to grow in the first 2 weeks dramatically by reduction of the immunity of the mother. So pregnancy disturbances and SMD share a common denominator that a reduction of HPA-axis in mental disturbances is mostly accompanied by a reduction in immunity of the patient as well. The reduction of the mother's immunity in the first three-months through reduction of the adrenals hormones of the HPA-axis of the mother, reduces her wellbeing: Most mothers complain in the beginning of a pregnancy about vomiting and an unusual moodiness.

It is also known that pregnancy increases the risk of developing schizophrenia, PTSD, or even increase severity of SMD.

Recent conducted human studies have also shown that a wide variety of prenatal stressors, from anxiety and partner relationship problems, to natural disasters, increase the risk for a diverse range of adverse neuro-developmental outcomes in the child. The impairment of maternal HPA axis can result in impaired cognitive development and behavioral problems, autism and schizophrenia, of the child just to name a few. The placenta can affect fetal neurodevelopment: It was shown for example in animal studies that prenatal stress can affect the activity of the placental barrier enzyme 11-betaHSD2, which metabolizes cortisol, a key hormone of the HPA-axis.

It has been suggested that schizophrenic and bipolar patients are more vulnerable to stress than healthy persons, and that stressors can trigger a psychotic episode or worsen symptoms, just as seen in pregnancy mediated via the HPA axis, which controls the release of cortisol. The relationship between the increase in schizophrenic and bipolar disorders severity is also quantitatively related to a decreased HPA axis sensitivity. [Hempel et al., Psychiatry Clin Neurosci. 2010 64:548-54.]

Increased Mortality and Cardiovascular Risk caused by Severe Mental Diseases Researchers have studied the health risks of individuals with mental illness and have unfortunately focused on socio-economic, disturbed social life and other factors and neglected that physiologic basis of these diseases, that improvements in seeing a doctor will not automatically improve the disease condition: There is no proof that patients with severe SMD have a higher life expectancy than those without medication. Compared with other populations, people with mental illness have a higher prevalence of cardiovascular risk factors, including smoking, overweight and obesity, lack of moderate exercise, harmful levels of alcohol consumption, excessive salt intake, and poor diet. Lack of emotional support and social networks, lower socioeconomic status, and substance abuse are

described as risk factors that affect mortality in people with serious mental illness. According to the Harvard Mental Health Letter, people with psychiatric disorders have higher rates of medical illnesses, but they often do not seek needed medical care.

Social consequences of mental illness include poverty, unemployment, poor housing,

stigma, and low self-esteem. Difficulties accessing health care include doctors' focus on mental illness and not physical health, erratic compliance with health screening and treatment, and poor communication.

Some mental health practitioners and heath care professionals are proposing ways to improve the physical health of individuals with mental illness, which could consequently help decrease mortality rates. If primary care and mental health professionals pay attention to the physical ramifications of mental illness, the physical health of people with serious mental illness can be improved. Improved intervention practices could include engaging clients in preventive care, diagnosis, and management of serious physical illnesses and additional training for mental and physical health professionals to encourage communication about patient care (Colton et al. Prey Chronic Dis 2005).

However, it is now more and more recognized among psychiatrist that the biological basis of severe cardiovascular risk is the unfavorable blood lipids and they will not be corrected by improved care and medication.

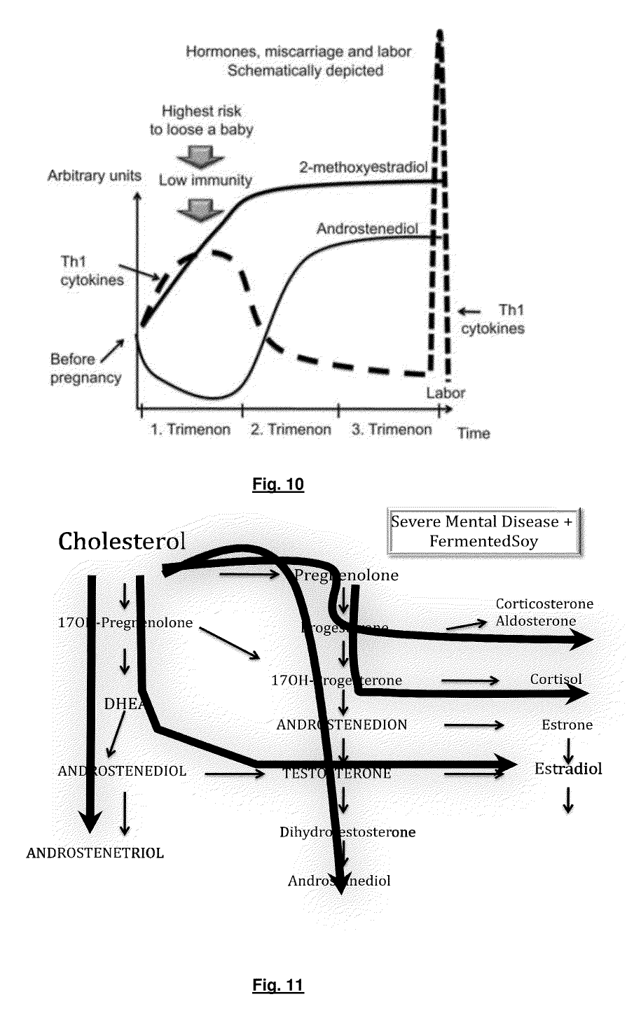

Second generation anti-psychotics improve some unfavorable conditions like reducing White Blood counts in schizophrenia patients, however worsen the unfavorable triglycerides even more (FIG. 13).

FIG. 13: Laboratory parameters where the percentage of abnormal values were significantly changed between the acute and recovery phases of Schizophrenia. Treatment was conducted with antipsychotics. Values are % patients presenting abnormal values. Abbreviations: K, potassium; WBC, white blood cells; TG, triglyceride; LDH, lactate dehydrogenase; UA, uric acid; FBS, fasting blood sugar; BUN, blood urea nitrogen. (Taken from: Bolton et al. Neuropsychiatric Disease and Treatment 2010:6 281-288)

It is therefore a major claim of our findings that soy in concentration similar to concentrations in soy baby nutritional food, based on body weight comparison, can restore treatment resistant blood lipids in SMD together with improving mental condition and normalizes WBC. This is in contrast to today's method of choice, where second generation anti-psychotics worsen blood lipids and triglyceride seven more (FIG. 13).

### Increase of White Blood Counts in Severe Mental Disease

As was shown above that stress impairs blood hematology in soldiers under normal training exercise. Patients with SMD are suffering even more from impaired blood hematology, where the differentiation of White blood cells is impaired, increasing the cell numbers. It has been described in the literature that soy ingredients have an impact on cell differentiation. However, it is claimed for the first time ever, that isoflavones, in concentrations similar to soy baby nutrition formulas, can normalize pathological WBC counts in patients with SMD, like schizophrenia.

The mechanism of action may be explained by the following mechanisms (FIG. 14). IL6 is normally expressed at low levels, except during infection, trauma, aging, or other stress conditions. Tumor necrosis factor (TNF-alpha)—induced IL6 gene expression is primarily controlled at the transcriptional level by the transcription factor nuclear factor-Kappa Beta (NF-KB).

After menopause or andropause, IL6 levels are increasing with age as a consequence of a rapid decline in circulating estrogen, or testosterone, or androstenes. This altered regulation may certainly account for several disease-associated inflammatory pathologies, phenotypical changes of advanced age, and accelerated tumorigenesis. With the aging of the population, prevention of these types of complaints and maintenance of the important physiologic inflammatory balance has attained paramount importance.

As is shown (FIG. 8), patients with severe mental diseases suffer from increased hormone levels at the upper parts of the sexual hormone level and a decreased hormone level at the lower part so that a decline of estradiol, testosterone, and particularly the androstenes will increase the inflammatory cytokines, IL-6, TNF-alpha to rise. It is textbook knowledge that cytokines like TNF-alpha and IL-6 can impair homeostatic particularly White Blood Counts (WBC) and lymphocytes (FIG. 14). It is interesting to note that cytokines can down regulate the differentiation of stem cells or progenitor cells, resulting in increased WBC, because they lack further differentiation.

Fermented soy products will not only down-regulate symptoms of severe mental disease but cytokines in the blood and will therefore normalize WBC and Lymphocytes.

FIG. 14: A schematic depiction of the relationship between androstenediol, and White blood counts and CD4 cells. With age, in infections the CD4 cells will go up.

### Water Retention Problem in Severe Mentally Disturbed Person

Impaired water excretion was noted to coincide with psychotic exacerbations in the first decades of the past century (Goldman, Brain Res Rev. 2009; 61:210-20). In the ensuing decades, life-threatening water intoxication and elevated plasma levels of the anti-diuretic hormone, arginine vasopressin (AVP) was reported in a subset of persons with schizophrenia. Subsequent studies demonstrated that the osmotic set point for AVP secretion was transiently reset in these patients by an unknown process and that this was further exacerbated by acute psychosis. More recent studies indicate that the AVP dysfunction is a manifestation of a hippocampus-mediated impairment in the regulation of both AVP and HPA axis responses to psychological, but not other types of, stimuli.

Severely mentally disturbed persons are suffering from edema in their legs, lung and other parts of the body. Lung edema can be so pronounced that they are suffering from drowning problem in their own water, which cannot be transported out of the body. Also, water retention will cause treatment resistant blood pressure. Aldosterone is a hormone that increases the re-absorption of sodium ions and water and the release (secretion) of potassium ions in the distal convoluted tubules of the kidneys. This increases blood volume and, therefore, increases blood pressure.

Drugs that interfere with the secretion or action of aldosterone are in use as antihypertensive. One example is spironolactone, which lowers blood pressure by blocking the aldosterone receptor. Aldosterone is part of the renin-angiotensin system. A measurement of aldosterone in blood may be termed a plasma aldosterone concentration, which may be compared to plasma renin activity. Aldosterone is produced by the outer-section (zona glomerulosa) of the adrenal cortex in the adrenal gland, and acts on the distal tubules and collecting ducts of the nephron, the functioning unit of the kidney to cause the conservation of sodium, secretion of potassium, increased water retention, and increased blood pressure. The overall effect of aldosterone is to increase reabsorption of ions and water in the kidney.

Its activity is reduced in Addison's disease and increased in Crohn's disease. Chronic schizophrenic patients are reported to develop imbalanced water homeostasis by the pathological secretion of vasopressin and aldosterone.

The presented biochemical and histomorphometric data suggest that genistein caused shunting of steroid metabolic pathways in the adrenals, supporting DHEA production and inhibiting corticosterone and aldosterone production in rats. (Ajdz et al. Exp Biol Med 234:148-156, 2009).

It is therefore the key finding of this patent that in concentrations similar to soy baby nutrition, comparison made on body weights, fermented soy is also decreasing DHEA in humans (FIG. 11), whereas in lower concentrations and in animal experiments, it tends to increase DHEA.

The reduction of the two stress hormones DHEA and pregnenolone is described herein for the first time. The reduction of aldosterone, a metabolite of pregnenolone, by soy isoflavones in doses similar to soy baby nutrition formulas, will not only decrease edema risk in the lungs but it will also reduce hypertension and decrease anxiety (FIG. 12). The exact mechanism why aldosterone is related to anxiety is not exactly known. It may be that it is only increased because it may increase if stress hormones are increased. It is possible that stress also causes edema and breathing problems, for example Spencer Tracy suffered from stress, and he had tremendous breathing problems, so that filming had to be suspended or postponed.

This finding helps creative and intellectuals, for example opera singers, who are under a lot of mental stress.

### Hyperprolactinaemia

The normal serum prolactin concentration is less than 500 mU/l (<15 ng/ml.). In women, the serum prolactin concentration increases to approximately 200 ng/ml during pregnancy, and reaches approximately 300 ng/ml during the onset of lactation. Prolactin concentrations decline after childbirth, even if breast-feeding continues, and within two to three months may attain near normal levels. (Frantz, Prolactin. N Engl J Med 1978; 298: 201-207).

Although prolactin is not a marker of schizophrenia, it is part of an observation, that breast-feeding and pregnancy, which increases prolactin levels, also increases schizophrenia attacks. Prolactin levels are elevated, however within the considered normal range, because hyperprolactinaemia can be a part of normal body changes during pregnancy and breastfeeding. Diseases may cause hyperprolactinaemia, affecting the hypothalamus or pituitary gland. It can also be caused by disruption of the normal regulation of prolactin levels by drugs, medicinal herbs and heavy metals. Hyperprolactinaemia may also be the result of disease of other organs such as the liver, kidneys, ovaries and thyroid. (Mancini, T. et al. (2008). “Hyperprolactinemia and Prolactinomas”. Endocrinology & Metabolism Clinics of North America 37 (1): 67.

Hyperprolactinaemia causes a reduction in sex drive, impairing the partner relationship. Therefore during periods of breast-feeding, women tend to reduce sexual contact with their partners. Severe mental diseases, besides schizophrenia like bipolar disorders or PTSD are related to an elevation of prolactin values to the upper boundaries of normal values.

Therefore, it is common complaint among psychiatrists, and known to experts in the field, that administration of antipsychotics of the first and second generation, which are commonly used to treat bipolar and schizophrenic patients, may cause hyperprolactinemia and impair partner relationships (Compton et al. Psychopharmacol Bull. 2002 36:143-64). Antipsychotics increase therefore unfavorable prolactin levels even further.

Also hyperprolactinaemia may cause disruptions in the normal menstrual period in women and hypogonadism, infertility and erectile dysfunction in men. Additionally antipsychotics may change body composition like producing gynecomastia and galactorrhea.

It is a major claim of this invention that the administration of fermented soy in concentrations similar to soy baby nutrition formulas, dose compared on body weight comparison, will not only improve the mental condition in severe mentally disturbed persons, but also reduce prolactin. It was observed that those patients started to develop to have a normal sexual relationship with their partners again.

Particularly, pediatricians are concerned that increased prolactin levels in children and adolescence by antipsychotics levels may cause physiologic changes like bone formation. (Roke Yet al. J Child Adolesc Psychopharmacol. 2009 19:403-14). It is a common fact that fermented soy is particularly bone protective, so that it is a major finding that fermented soy, in doses compared to baby nutrition formula compared by body weight, prevents bone decay and simultaneously increases mental health in children, adolescence and adults, avoiding the side effects of antipsychotics for the first time ever. This claim holds for children, adolescence and adults.

Soy Isoflavones have Only Modest Effects on Sexual Steroids in Low Doses.

Clearly the most important finding is that higher daily doses of soy isoflavones are required, like in baby soy nutrition formula, to elicit effects on sexual hormone metabolism (FIG. 11).

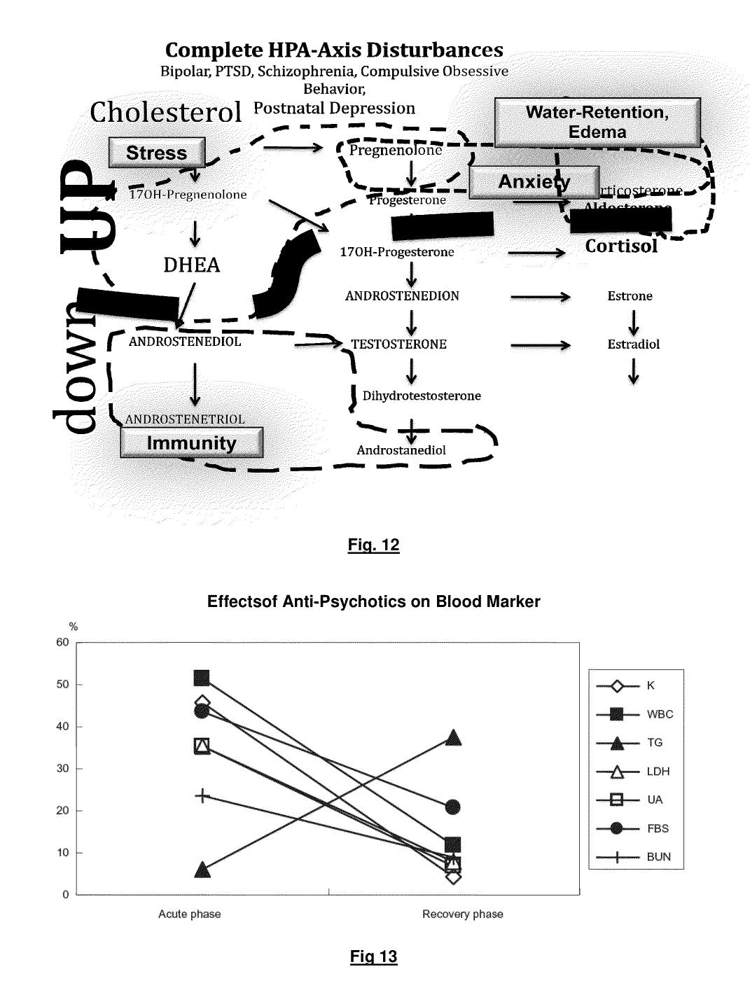

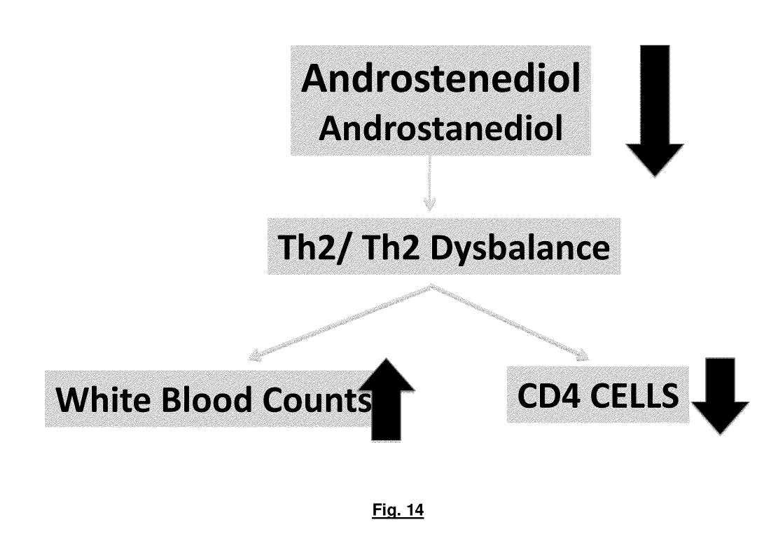

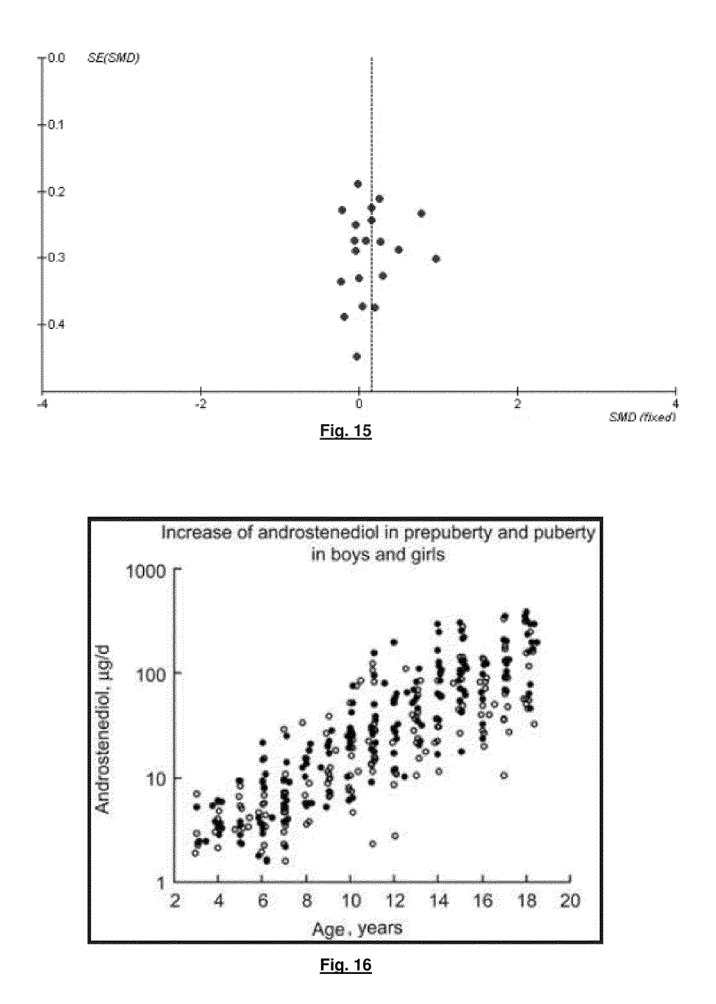

It is known to the expert that low dose soy isoflavones up to 120 mg per person per day have insufficient influence on the hormone metabolism (FIG. 15) to produce favorable effects in severe mental disease, although some effect have been observed in menopause and peri-menopause. A total of 28 Japanese healthy volunteers between 30 and 59 years of age were given soy isoflavones (60 mg daily) supplements for 3 months, and the changes in their sex hormone levels were investigated at the baseline and after administration (Tanaka et al, Prostate Cancer Prostatic Dis. 2009; 12:247-52). No changes in the serum levels of estradiol and total testosterone were detected after 3-month supplementation. The serum levels of sex hormone-binding globulin significantly increased, and the serum levels of free testosterone and dihydrotestosterone decreased significantly after 3-month supplementation.

In another study in menopausal women receiving 120 mg soy isoflavone, total testosterone and HDL levels were significantly lower in the isoflavones compared to placebo groups (Basaria et al, J Endocrinol Invest. 2009 32:150-5).

FIG. 15: Funnel plot of results from included published studies on the effects of low doses of soy protein and isoflavones (up to 120 mg) on circulating total estradiol (E2, pmol/l) in post-menopausal women (From:Hooper et al, Hum Reprod Update. 2009; 15: 423-440).

The most rigorous review was concerning this question was undertaken by Hooper (Hum Reprod Update. 2009; 15:423-40.). They concluded that in post-menopausal women, there were no statistically significant effects on estradiol, estrone, SHBG, FSH or LH, although there was a small statistically non-significant increase in total estradiol with soy or isoflavones (approximately 14% (FIG. 15).

A recently published study from China (Li Y, et al. Wei Sheng Yan Jiu. 2010 January; 39(1):56-9.) where soy is consumed in the traditional form of fermented soy, did reveal estradiol, prolactin and testosterone were slightly increased and FSH, was decreased. These effects however are too small to elicit pharmacologically effects in severe mental diseases. It can generally be concluded that soy effects on sex hormone metabolism is limited.

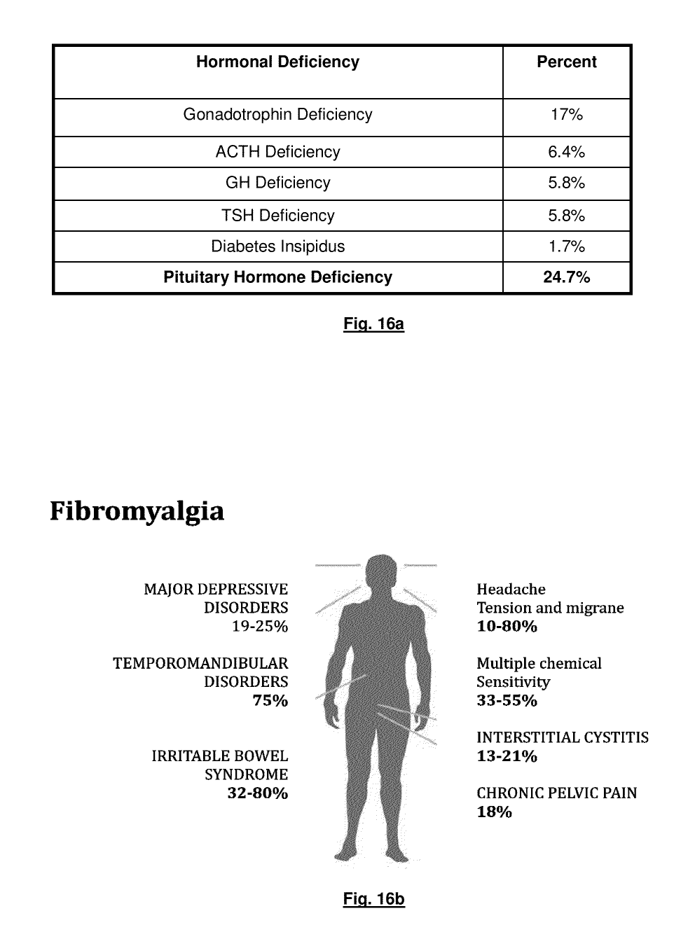

Why did soy baby nutrition formula not produce similar favorable effects as those seen in adolescence and adults? Clearly the hormone system and immunity of babies is completely underdeveloped, and is developed in the adrenarche, where adrenal hormones strongly increase (FIG. 16). This is an insensitivity mechanism, which prevents babies during pregnancy from being rejected by their mother's body. The baby pays for the “life-saving” insensitivity during pregnancy with an increased risk of developing allergies and neurodermatitis as an infant (Rohr et al 2010).

FIG. 16: Relationship between androstenediol and age in boys (O) and girls (□).

Taken from (Rohr et al Horm Mol Biol Clin Invest 2010; 3(2)).

### Temporary Brain Injury

An estimated 1.9 million Americans sustain a Temporary Brain Injury (TBI) each year. Half of these cases result in at least short-term disability, and approximately 52,000 of those people die on the spot. About 300,000 end up in a hospital, of those that survive, many will develop progressive hormonal deficiencies, which leads to post-TBI Hormone Dysfunction Syndrome. This cascade of hormonal deficiencies and insufficiencies will affect every aspect psychological, physiological and physical functioning and will have a dramatic effect upon the patient's quality of life. Falls (account for 28%); Moving Vehicle Accidents (20%); Struck by/against events (19%); and Assaults (11%).360,000 Veterans May Have Traumatic Brain Injury.

It was written in USA Today 2009 that Pentagon officials estimated for the first time that up to 360,000 Iraq and Afghanistan veterans may have suffered brain injuries. Among them are 45,000 to 90,000 veterans whose symptoms persist and warrant specialized care.

Army Brig. Gen. Loree Sutton provided the estimate during a news conference around March, 2010 as Brain Injury Awareness Month. She heads the Pentagon's Centers of Excellence for Psychological Health and Traumatic Brain Injury. Ref: Biomarkers of proteolytic damage following traumatic brain injury. (Pineda, Brain Pathol 2004; 14(2):202-9.)

Two new biomarkers; Caspase and Calpain are specific for TBI. These are responsible for initiation of Apoptosis and subsequent cell death. Focal areas of damage are: the Cortex, Cerebellum, Thalamus (Hypothalamus), Hippocampus. There is a prevalence of Hypopituitarism and growth hormone deficiency in adults long-term after severe traumatic brain injury (Vorgem del Rocio Clinical Endocrinology 2005 May; 62(5):525-32).

FIG. 16a: HPA marker in fibromyalgia

Alterations in prolactin levels due to TBI have been described in several studies.3,5,7 Prolactin is synthesized and released by lactotroph cells in the anterior pituitary. Its main function is to stimulate lactation in postpartum women; however, it is increased by pituitary stalk compression and by many medications. In contrast to other pituitary hormones, prolactin is under tonic inhibitory control from the hypothalamic dopamine neurons.66ln addition to stalk injury and medications, stress, suckling, and the thyroid stimulating hormone (TSH) modulate prolactin secretion. Sertonergic pathways activate prolactin release, explaining why some selective serotonin reuptake inhibitors (SSRIs) may lead to hyperprolactinemia. Elevated prolactin, in turn, modulates other endocrine systems. Hyperprolactinemiais associated with inhibition of GnRH-induced LH secretion and suppression of the gonadal axis.

Another concern is the increase of adrenal hormones and the decrease of testosterone. This has detrimental effects on immunity, because the metabolite androstanediol cannot be produced.

The finding that a subfraction of severe TBI with HPA disturbances can improve dramamtically when consuming fermented soy containing a concentration higher than 300 mg of soy isoflavones, is a finding of this patent. Those hormones, which are elevated, like cholresterol, proplactin and DHEA, and pregnenelone can be decreased and their metabolites can help the healing. It increases particulalry Testosteroneand and Estradiol, which have strong effect on wellbeing.

### HPA Axis of the Energy Crisis Disorders

Diseases of the energy crisis disorders are
Fibromyalgia
Chronic Fatigue
Thyroid Disorders
Adrenal Fatigue
Autoimmunity

These energy crisis disorders are related to a hypothalamic, pituitary, adrenal, thyroid, gonadal, and gut axis dysregulation. These disorders are tightly intertwined and often difficult to distinguish. These dysfunctions may lead to multiple hormone abnormalities that may affect the natural circadian rhythms of the body such as the sleep wake cycle, rhythmic hormone secretions involving growth hormone, thyroid, gonadal hormones, gut hormones, adrenal hormones, as well as a loss of healthy immunological surveillance.

FIG. 16b: Schematic depiction of comorbidity of fibromyalgia.

Soy in normal concentration in Asian or in Western consumption of up to 100 mg/day does not improve the condition. A recently conducted review about Evidence for the efficacy of complementary and alternative medicines in the management of fibromyalgia: a systematic review, came to the conclusion that soy in low doses does not improve the conditions of fibromyalgia (de Silva et al, Rheumatology. (Oxford). 2010 June; 49(6):1063-8.

Fibromyalgia is associated with disabilities. Common medication, are
Nsaids, cox-2 inhibitors
nAnti-convulsants
TCAs
Opioid
Muscle relaxants
Tramadol
SSRIs and SNRIs

There is a strong stress response to fibromyalgia. “Poverty” Alters HPAT Function

The stress-disequilibrium theory: chronic disease, low social control and physiological

changes in the HPA axis (R Karasek, Med Lay 2006; 97: 258-72). Perceived loss of “Locus of Control”Low feeling of esteem, attribution, love or purpose associated with altered HPAT axis plasticity.

There is strong evidence that the HPA axis is involved in the etiology of fibromyalgia: Cytokines and HPA Axis Alteration are persistent in fibromyalgia: IL-6, a cytokine that is an important modulator of the HPAT stress response is elevated (Mastorakos, Ann NY Acad Sci 2006; 1088: 373-81). There are many reports between raised stress and immune parameters in fibromyalgia. Increased cytokines can produce sickness, behavioral problems and fever.

It has been established for a long time that their Estrogen metabolism was found to be 10 times lower than comparisons to normal people. Urinary concentrations of the anti-estrogens, 2-hydroxyestrone and 2-hydroxyestradiol, were found to be 10 times lower in patients with RA and SLE than in controls, and the ratio of 2-hydroxyestrogens to 16-hydroxyestrogens was 20 times lower than controls.

It does not come as a surprise that fermented soy product higher than 500 mg isoflavones per day did improve symptoms of fibromyalgia, and also the reduced metabolism of 2-hydroxyestradiol. Although it has been known for a long time that soy in normal concentrations favors estradiol metabolism, it has not been observed that soy isoflavone in concentrations preferably between 500 to 1000 mg/day do eradicate stress, and symptoms in fibromyalgia patients.

### Stress as Cause of Epilepsia

It has been long been known that early life stress predisposes to the development of psychiatric disorders (Gross, Hippocampus. 2010) In this context the hippocampal formation is of particular interest, because it is affected by stress on the structural and cognitive level. Stress is mediated via HPA axis and it is not fully understood how soy isoflaovnes in concentrations between 500 to 1000 mg reduces stress, modulates cortisone and balance epilepsia.

It is known that testosterone can reduce epilepsia (Frye Epilepsy Behav. 2009 November; 16(3):418-22). Testosterone (T), the principal androgen secreted by the testes, can have antiseizure effects. Some of these effects may be mediated by T's metabolites. T is metabolized to 3alpha-androstanediol (3alpha-diol). T, however, not 3alpha-diol, binds to the androgen receptor. It has been shown that Estrogens with activity at ER-beta, but not those selective for ER-alpha, produced antiseizure effects.

Since soy isoflavones, however only in concentrations higher than 300 mg, preferably higher than 500 to 1000 mg, increase testosterone and 3-alpha-diol it was found that fermented soy has an excellent profile to prevent epilepsia.

### Protease Inhibitor

Since a specially fermented soy formulation is completely altering mental as well as physical symptoms, we noticed that the immune modifying agent BOWMAN BIRK INHIBITOR FACTOR is necessary to improve the immunity of persons suffering form mental disease. The BOWMAN BIRK INHIGITORY FACTOR is known to improve immunity, however it is the first time ever that it was proven successful in patients suffering form severe mental diseases, which are defined and subject in this patent. Protein inhibitors are now a well established class of chemopreventive agents (Kennedy et al 1998). Besides carcinogenic effects they show strong immunomodulating effects). There are several protease inhibitors that are part of the human diet that have the ability (Kenendy et al 1998) to interfere with chymotrypsin or chymotrypsin like enzymes. One potent protease inhibitor is Bowman Birk Inhibitor (BBI), has been studied extensively.

The plant and animal kingdom contain a variety of protease inhibitors. They are found in many common foods, including legumes, cereals, oilseeds, nuts, fruits, vegetable, eggs, potatoes, and other dairy and animal products. Many vegetables contain protease inhibitors (Birk 1974, 1976, 1993, Liener and Kakadec, 1980, Rackis et al, 1986) Soybeans are usually rich in protease inhibitors activity, where at least 6% of the total soybean protein (Hwang et al 1977, Rackis and Anderson, 1964). The best characterized of these inhibitors are presenting soybean trypsin inhibitor (SBTI) (Kunitz, 1977; and BBI (Bowman, 1946, 1993, Birk, 1961, 1974, 1976, 1985) SBTI has a molecular weight of about 21000 and has a primarily trypsin inhibitor (TI) activity; BBI has a molecular weight of 8000 and inhibits chymotrypsin and trypsin (Birk 1974, 1976) Other soybean protease inhibitors have not been as fully characterized as BBI and SBTI, but are known to inhibit trypsin (Hwang et al. 1977)

The following table lists some common known protease inhibitors and references in the literature. The most potent protease inhibitor by far is Bowman Birk Inhibitory Factor.

TABLE
Protease inhibitors with immune modulating effects.

| Protease inhibitor | First reference |
|---|---|
| Antipain | Kennedy and Little, 1978 |
| Leupeptin | Kennedy and Little, 1978 |
| Antipain | Borek et al, 1979 |
| Antipain | Kuroki et al, 1979 |
| Chymostatin | |
| Elastatinol | |
| Leupeptin | |
| Pepstatin | |
| Antipain | Kennedy and Weichselbaum, |
| | 1981 |
| Leupeptin | |
| BBI | |
| Chymostatin | Kennedy 1984, 1985 |
| TPCK | Kennedy 1985a |
| BBI (fragment containing chymotrypsin | |
| inhibitory sites) | |
| BBIC | Yavelow et al, 1983, 1985 |
| | Baturay and Kennedy, 1986 |
| Chickenpea inhibitor | Yavelow, 1985 |
| Chymotrypsin inhibitors I from potatoes | Billing et al, 1987 |
| Carboxypeptidase inhibitors I and II from | Billing et al, 1989 |
| potatoes | |
| Aprotinin and N-acetyl-L tyrosine ethyl | Billing et al, 1989 |
| ester | |
| BBI-polylysine conjugate | Persiani et al, 1991 |
| BBI | St Claire, 1991 |
| Edi Pro A (soybean extract prepared by | Kennedy, 1993 |
| Ralston Purina) | |
| E-amino-n-caproic acid | Kennedy, 1993 |
| Succinylated BBI-BBI lacking TI site | Kennedy, 1993 |
| Spermine-BBI conjugate | Ekramini et al, 1993 |
| BBI-polyester conjugate | Larinova et al., 1994 |
| BBI-palmitic acid derivatized conjugate | Erami et al., 1995 |

Table taken form reference: Ann Kennedy. Protease inhibitors. Pharmacol Thert 1998: 78; 167-209

### DETAILED DESCRIPTION OF THE INVENTION

It has now been discovered that special isoflavone containing soy formulations, fermented soy, red clover, alfalfa, and other isoflavone sources, either by natural or synthetic origin modulates the stress systems and/or the actions of CRF and/or glucocorticoids (GC), thereby presenting novel pharmacotherapeutic options. As demonstrated herein, that special isoflavone containing soy formulations, fermented soy, red clover, alfalfa, and other isoflavone sources, either by natural or synthetic origin administration appears to reduce or protect against stress and it effects (e.g., anxiety, obsessive-compulsive behavior, depression, psychosis, changes in eating behavior) and is extremely effective reducing inflammation and infections, as well as reduce metabolic disorders. We also demonstrate herein that, special isoflavone containing soy formulations, fermented soy, red clover, alfalfa, and other isoflavone sources, either by natural or synthetic origin administration is capable in humans to results in behavioral effects that include anti-stress, anxiolytic, antidepressant, and antipsychotic actions.

In exploring new medical treatments, it was noted that a number of metabolic pathologies (e.g., diabetes, obesity) are associated with behavioral dysfunction (e.g., major depression, schizophrenia, bipolar disorders). Although these diseases are generally believed to be co-morbid, there is recent evidence suggesting that the behavioral and metabolic alterations are physiologically linked in many cases[24-26]. A common link between these seemingly disparate disease states may be chronic stress and the associated changes in brain CRF and the adrenocortical steroid hormones, GC.

The CRF and GC molecules play a critical role in modulating behavioral, neuroendocrine, autonomic, and metabolic function under normal and stressful conditions. Chronic stress and the induction of expression and activity of these molecules are highly associated with behavioral diseases like anxiety and depression, and also with some obesities and diabetes. There is evidence that links CRF and adrenocortical abnormalities to the metabolic syndrome, autoimmune inflammatory disorders, acute and chronic neuro-degeneration, sleep disorders, chronic pain, eating disorders, chronic anxiety disorder, and major depression [27-30]. As demonstrated herein, fermented soy is shown to share properties of anxiolytic, antidepressant, and antipsychotic agents in human behavioral testing. Thus, it has now been discovered that fermented soy may have the surprising ability to treat psychiatric disorders. Psychiatric disorders that can be treated include anxiety disorders, schizophrenia and other psychotic disorders, substance-related disorders, sleep disorders, somatoform disorders, and eating disorders. These compounds may be particularly effective in treating severe psychiatric disorders that have elements of metabolic disturbances, e.g., eating disorders, or in treating patients with a psychiatric disorder or those with a psychiatric disorder and who also suffer from a metabolic disturbance.

More particular types of the above named disorders can be found in the DSM-IV.

The following are only examples of disorders that may be treated by the methods disclosed herein. Examples include mood disorders that may include depressive disorders and bipolar disorders. They can further be characterized as major depressive disorders, dysthymic disorder, bipolar I disorder, bipolar II disorder, cyclothymic disorder, bipolar disorder not otherwise specified, mood disorders due to a medical condition, substance-induced mood disorder, or mood disorder not otherwise specified. Anxiety disorders can include panic disorder, specific phobia, social phobia, obsessive-compulsive disorder, posttraumatic stress disorder, acute stress disorder, generalized anxiety disorder, anxiety disorder due to a medical condition, substance induced anxiety disorder and anxiety disorder not otherwise specified.

Substance-related disorders include substance dependence, substance addiction, substance-induced anxiety disorder, and substance-induced mood disorder. Substance dependence and addiction can occur with a variety of substances, including but not limited to, alcohol, nicotine, cocaine, opioids, narcotics, hallucinogens, amphetamines, phencyclidines, phencyclidine-like substances, inhalants, and sedatives. Substance-induced anxiety disorder can occur in response to substances, which include, but not limited to, caffeine, cannabis, cocaine, hallucinogens, amphetamines, phencyclidines, phencyclidine-like substances, and inhalants. Substance-induced mood disorder can occur in response to substances, which, include, but not limited to cocaine, hallucinogens, opioids, amphetamines, phencyclidines, phencyclidine-like substances, and inhalants. Substance-related disorders can occur in response to one substance or to a combination of substances, such as in polysubstance related disorder.

In certain embodiments, methods provided may not include the treatment of somatoform disorders. In certain embodiments, methods provided might include somatoform disorders but do not include the treatment of physical pain. In still other embodiments, methods provided may include the treatment of the psychiatric illness associated with pain.

In one general aspect, it is claimed that special isoflavone containing soy formulations, fermented soy, red clover, alfalfa, and other isoflavone sources, either by natural or synthetic origin that reduce or moderate stress, or regulate the stress pathway, may be useful as pharmacotherapeutic agents. In another general aspect, it is claimed that special isoflavone containing soy formulations, fermented soy, red clover, alfalfa, and other isoflavone sources, either by natural or synthetic origin that can affect or regulate metabolic disturbances as well as psychiatric or behavioral processes would be useful. In another general aspect, it is claimed that special isoflavone containing soy formulations, fermented soy, red clover, alfalfa, and other isoflavone sources, either by natural or synthetic origin can attenuate or reverse metabolic disturbances would be useful as treatments of psychiatric diseases or disorders. It is claimed that fermented soy is beneficial in the methods provided. It is claimed herein, for the first time, that isoflavone, in concentrations found in baby soy formulas, when consumed in high doses, proportional to body weight, not only treats the psychiatric illnesses, but also alleviates the physical co-morbidities of the illnesses and elicits an increased rate of treatment response and outcome success in patients with a psychiatric illness. Physical co-morbidities, like diabetes, infections, inflammation, exacerbate the morbidity that comes with psychiatric illness and lead to a reduction in treatment response. Special isoflavone containing soy formulations, fermented soy, red clover, alfalfa, and other isoflavone sources, either by natural or synthetic origin may be particularly useful in the methods described herein because of its anti-diabetic, anti-stroke, anti-infarction, as well as anti-inflammatory, and antiviral and antibacterial effects are useful.

These effects may increase the rate of treatment response and outcome success in certain patient populations who suffer a psychiatric illness and who exhibit diabetes, risk of metabolic disease, infarction, infection, virus infection, inflammation (e.g., diabetes, gastritis, lung inflammation, Cushing's syndrome, Cushing's disease, atypical major depression, schizophrenia, seasonal affective disorder, polycystic ovary syndrome, night eating syndrome, bulimia nervosa, binge eating disorder, and chronic fatigue syndrome). In certain embodiments, the methods do not include treatment of anorexia. In other embodiments, the methods include treating the psychiatric illness associated with anorexia.

It is further contemplated that special isoflavone containing soy formulations, fermented soy, red clover, alfalfa, and other isoflavone sources, either by natural or synthetic origin may be used in conjunction with other psychiatric medications or therapies, such as those conventionally used to treat psychiatric disease, such as tricyclic antidepressants and the monamine oxidase inhibitors (MAOIs), selective serotonin reuptake inhibitors (SSRIs), serotonin and noradrenaline reuptake inhibitors (SNRIs), herbal antidepressants (e.g., St John's Wort or Hypericum), second generation antipsychotic medications, psychoanalysis, cognitive-behavioral therapy, and interpersonal therapy. For example, special isoflavone containing soy formulations, fermented soy, red clover, alfalfa, and other isoflavone sources, either by natural or synthetic origin may be administered during the same time period as the other psychiatric medication, during an overlapping time period as the other psychiatric medication, or in a time period that does not overlap with administration of the other psychiatric medication. As a combination or add-on therapy, the beneficial qualities of special isoflavone containing soy formulations, fermented soy, red clover, alfalfa, and other isoflavone sources, either by natural or synthetic origin may counteract or moderate one or more of the unwanted side effects of currently available medications, e.g., diabetes, increasing metabolic diseases.

For example, second generation antipsychotics (SGAs) are effective therapeutics for the treatment of symptoms associated with schizophrenia and related psychotic conditions. Despite these advances in treating the psychiatric condition, accumulating clinical data have revealed an association between the use of SGAs, diabetes, and dyslipidemia dysglycemia, hypertension, elevated hepatic transaminases and prolactin levels have been reported[31,32]. Exemplary SGAs such as clozapine and olanzapine have been identified as being likely to produce dizziness; in addition, these two SGAs have also been associated with increased risk for both diabetes and dyslipidemia. In addition, special isoflavone containing soy formulations, fermented soy, red clover, alfalfa, and other isoflavone sources, either by natural or synthetic origins also able to treat or aid in the treatment of diabetes and dyslipidemia. Accordingly, when used with other psychiatric medications, fermented soy may not only provide an additional treatment to the psychiatric condition, but also be able to counteract at least a negative side effect of those other psychiatric medications.

Patients can be of any age. Accordingly, these disorders can be found in young adults and adults (defined herein as those aged 65 or under) as well as infants, children, adolescents, and the elderly (defined herein as over the age of 65). In fact, certain segments of the population may be particularly prone to having a particular condition, such as eating disorders in adolescents and young adults. The elderly may be particularly susceptible to co-conditions such as depression, diabetes, and dyslipidia.

As used herein, and as well understood in the art, “treatment” is an approach for obtaining beneficial or desired results, including clinical results. “Treating” or “palliating” a disease, disorder, or condition means that the extent, undesirable clinical manifestations of a condition, or both, of a disorder or a disease state are lessened and/or time course of the progression is slowed or lengthened, as compared to not treating the disorder. For purposes of the methods disclosed herein, beneficial or desired clinical results include, but are not limited to, alleviation or amelioration of one or more symptoms, diminishment of extent of disorder, stabilized (i.e., not worsening) state of disorder, delay or slowing of disorder progression, amelioration or palliation of the disorder, and remission (whether partial or total), whether detectable or undetectable. “Treatment” can also mean prolonging survival as compared to expected survival if not receiving treatment. Further, treating does not necessarily occur by administration of one dose, but often occurs upon administration of a series of doses. Thus, a therapeutically effective amount, an amount sufficient to palliate, or an amount sufficient to treat a disease, disorder, or condition may be administered in one or more administrations.

Special isoflavone containing soy formulations, fermented soy, red clover, alfalfa, and other isoflavone sources, either by natural or synthetic origin exert its psychiatric/behavioral effects by modulating the HPA axis, as well as biosynthesis of steroidal hormones in the brain and impact functions that are normally modulated by

### a) Cytokines,

b) sexual hormones,
c) hormones of the HPA axis, (e.g., CFR) expression and activity, behavior, autonomic nervous system activity, neuro-endocrine function, and metabolism). Stress, GC, and CRF seem to have an intricate and complicated relationship in psychological and metabolic function. Stress has profound effects on neuro-endocrine (e.g., hypothalamic-pituitary-adrenal (HPA) axis), autonomic, behavioral (e.g., anxiety, depression, substance abuse, feeding), and metabolic (e.g., fat deposition, energy utilization) function in man and animals [26]. All of these effects are modulated by GCs. The neuropeptide CRF mediates many of the stress-induced responses, including acute inhibition of food intake and anxiety [33,34]. GC and CRF activity are tightly interdependent and, together, make up a functional and well-described physiological system that controls behavioral, autonomic, neuro-endocrine, and metabolic function[35].

Hyper- and hypo-cortisolemia are also well-documented features of psychiatric disease, and patients suffering from the metabolic syndrome show signs of abnormal cortisol secretion and are more likely to present with a psychiatric disease such as major depression. Chronic stress and the associated increase in central CRF activity are believed to play a critical role in the development of clinical depression, anxiety disorders, substance abuse, eating disorders, and metabolic syndrome[36]. In fact, small molecule CRF antagonists are currently being investigated for their therapeutic actions in patients with major depression, general anxiety disorder, eating disorders, and other stress related pathologies.

It has been more and more recognized that severe mental disturbances are accompanied by a Th1/Th2 cytokine modification. Almost all patients suffering from increased Th2 cytokines like TNF-alpha. At the same time Th1 cytokines, like IL-2, which are normally responsible for immunity are reduced.

Severe mental diseases are characterized by immunity disturbances (like increased Th2 cytokines) and at the same time show low immunity (decreased Th1 cytokines). Patients suffering from severe mental diseases need to balance cytokines. Currently there are no drugs, which can increase Th1 cytokines and reduce Th2 cytokines, simultaneously, i.e. increase anti-infective immunity and decrease inflammation. Hormonal steroid, which are coined as androstenes (androstenediol and androstanediol, (FIG. 9) increase the levels of the Th1 cytokines such as IL-2, IL-3, and IFN, and decrease Th2, which are related to inflammation. Cortisone suppresses inflammation, but increase the risk of infection. Cortisones, suppress cellular immunity, so that viral and bacterial infection risk are increased.

Androstenes are locally metabolized in the brain, skin etc. either from DHEA or testosterone. Interestingly almost all patients who are suffering from decreased testosterone levels, do so because their metabolism is impaired, as well as decreased androstenes, because DHEA metabolism is impaired.

In the literature it is mentioned that fermented soy can be used to balance TH1/Th2 cytokines in children suffering from peanut allergy. There is no indication in the literature that fermented soy is balancing cytokines in severe mental diseases into the beneficial direction, Decrease Th2 and increase Th1. One has to remember that about 20 cytokines can be identifies per Th1 or Th2 fraction of cytokines.

Surprisingly, it was found that special isoflavone containing soy formulations, fermented soy, red clover, alfalfa, and other isoflavone sources, either by natural or synthetic origin product could be used to abolish
1) mental stress, sleeping disturbances, anxiety, depression, and emotional numbness, psychotic behavior, and schizophrenia,
2) immune disturbances, like herpes infections in patients suffering from severe mental disturbances.
3) abolish inflammation like gastritis, joint pain, pain in patients suffering from severe mental disturbances.
4) development of hypertension, diabetes, and metabolic disease in patients suffering from severe mental disturbances.
5) aggression, particularly suicide and murder attempts, which result from stress and mental disturbances described under point 1) in patients suffering from severe mental disturbances.

The exact common basis is still undetermined, however, Th1 and Th2 cytokines, which cause and are involved in many immune diseases, can cross the blood brain barrier. Circulating mediators of inflammatory and immune responses have been considered as potential modifiers of CNS responses since the experiments of Besedovsky and colleagues in the 1970's. These achieved particular attention when it was demonstrated that interleukin (IL 1) could reproduce some of these actions and activate the HPAA in particular. Interestingly, cytokines are often considered not to be true hormones, although they clearly meet the necessary criteria. Certainly most cytokines do not seem to have an endocrine function, although some do (Table 3). The lack of clarity over their endocrine role has been a particular source of confusion in considering the role of cytokines in inflammation, especially in terms of explaining effects on the CNS. This issue is clearly compounded if one cannot localize where the inflammatory stimulus that is inducing these hormones is itself acting. Including some 50 chemokines, and 30 interleukins, the number of characterized cytokines is now well in excess of 100. Many of these are particularly associated with regulation of the adaptive immune system and anti-viral responses, or act as growth and repair factors, and the great majority act primarily within the tissue where inflammation arises. However, despite their number, the focus in terms of the responses under discussion has remained quite firmly with IL-1, IL-6 and tumor necrosis factor α (TNF-α).

TABLE 3
Exemplar Actions of ‘Inflammatory’ Cytokines

| Locus of | | | | |
|---|---|---|---|---|
| Action | Biological Action | TNF-alpha | IL-1 | IL-6 |
| Site of Tissue | Inflammation COX-2 and | + | + | +− |
| | iNOS synthase induction | | | |
| | Endothelial cell activation | + | + | ++ |
| | Proteolytic enzyme | + | ++ | − |
| | induction Cytokine induction | + | ++ | − |
| Lymphoid | Lymphocyte regulation | ± | ± | + |
| Tissues | | | | |
| Systemic | Acute phase protein | + | ++ | ++ |
| | induction CRH/ACTH/ | ± | ++ | + |
| | glucocorticoid | + | ++ | + |
| | Induction of fever | + | − | + |
| | Cachexia/anorexia | ± | + | ++ |
| | Myelopoiesis | | | |

Table taken from ref. [42].

It can be deduced from Table 3 that general inflammation patients suffering from SMD is different from general inflammation. Most PTSD, schizophrenia patients show increased TNF-α in the blood circulation. SMD patients suffer from cachexia, appetite loss, however they do not suffer from fever. If one suffers from viral infection, macrophage activation will produce release of IL-6 and IL-1, resulting into appetite loss and fever.

Whether triggered by infection or physical trauma, cells of the monocyte/macrophage lineage are generally the first to respond and produce these cytokines. They also produce the only natural and well characterized competitive cytokine antagonist, IL-1 receptor antagonist (IL-1rα) and liberate soluble forms of the TNF-α and IL-1 receptors that are able to bind and neutralize TNF-α and IL-1 respectively. A soluble form of the IL-6 receptor is also produced (sIL-6R), although it does not neutralize IL-6 and binds it in a biologically active state. IL-1 and TNF-α are able to induce further IL-6 production in surrounding connective tissue and endothelial cells, which explains why IL-6 is almost always found in far higher concentrations than TNF-α or IL-1 during an inflammatory response. As suggested above, IL-1 and TNF-α are almost never found in biologically significant amounts in plasma, unless the stimulus (such as LPS) is present systemically.

This observation has consequences for SMD patients, since any increase in TNF-α will ultimately induce inflammation in the endothelium of the blood vessel. Therefore all patients suffering from SMD, face the risk to develop hypertension. Also, cytokine increase like TNF-α in connective tissues will cause pain, a very frequent complaint in patients suffering from severe mental diseases, e.g. fibromyalgia, gastritis, pneumonia, etc.

The innate immune system of the CNS is represented by microglia cells rather than by macrophages. There are, however, also macrophages around many of the blood vessels and these are certainly capable of producing cytokines such as IL-1 and IL-6. Production by these cells is one of the few ways that these cytokines can enter the CNS, since the blood brain barrier (BBB) excludes entry of such proteins. An exception is in regions where the BBB is not well formed, such as around the circumventricular organs (CVOs), the meninges and the choroid plexus. Transport of cytokines across the BBB has been described, but this is at such a low level it cannot be considered a realistic means of entry, unless plasma cytokine concentrations are maintained at a high concentration, and for most cytokines this is not the case. Although cytokines may not cross the BBB in total, they may enter hypothalamus. There is not any doubt that low androstenediol, and one of the two androstenes, may trigger cytokine release in microglia cells in the brain. IL-1, TNF-, and Il-6 have been identified in hippocampus, and hypothalamus. In animal models androstenediol has been found to reduce stress via activity in hypothalamus and hippocampus. In both regions microglia cells exists as well can be reached by cytokines in the microcirculation in the hypothalamus and hippocampus.

A key cytokine in mediating CNS effects is IL-6. Patients suffering from PTSD only a few months up to two years may not show any increase of IL-6 in the peripheral blood. If the disease prevails, depression will automatically develop and as a consequence, IL-6 levels as well. In our sample of soldiers suffering from PTSD from less than a year, we were not able to detect high IL-6 concentration in the peripheral blood. There may be two reasons for this. The half life of IL-6 in blood is relatively short compared to TNF-α, and secondly IL-6 may be produced only locally in the brain. IL-6 in the systemic blood circulation is related to depression. PTSD is not accompanied by depression. In fact, in NATO-ISAF-soldiers, suffering from PTSD only several months to maximum a year, depression was not detectable. PTSD patients will develop more severe inflammation and this may be accompanied by depression. In our own clinical studies with cancer patients who were given up by their physicians as untreatable and unresponsive to chemotherapy anymore, high IL-6 systemic levels were accompanied by depression.

Further references for the relationship between depression and immunity and Th1 cytokines can be found in ref [7].

Supporting evidence that circulating IL-6 is not involved in direct actions in PTSD patients in the beginning of the disease is corroborated by two clinical findings: First, missing signs of fever and second, missing depression symptoms in these PTSD patients suffering for only a short time. In fact, IL-6 acts via the HPAA (Hypothalamic-pituitary-adrenal axis), the classical depression axis in the body. Our conclusion is corroborated by the observation that cortisone values are not increased, which is normally seen if depression occurs.

Fermented soy is used in women around menopause, to reduce symptoms caused by reduced ovarian hormones. The study in soldiers is important, because they are suffering from anxiety and stress disorders, which are related to HPA axis disturbances. This is different from menopausal women, who have not suffered from major depression. Therefore it is surprising that a fermented soy formulation is effective in severe mental diseases.

This patent relates to the common knowledge that a relationship exists between stress and immunity. Recent investigation reveals that infections of the lung, stomach, as well as airway diseases are caused by a decline of Th1 cytokines, like Interferon-γ. It is common knowledge that Heliobacter pylorus is the major factor in gastritis, in which a decline of Th1 cytokines is a major force in the development of the disease. Patients suffering from severe mental disease, frequently suffer from airway diseases, lung infections as well as gastritis.

Hepatitis A, B, C viruses reduce Th1 cytokines or a reduction of Th1 cytokines increase hepatitis risk. Patients suffering from severe mental disease, suffer from an 11 to 20 times higher risk developing hepatitis infections.

Psychotropic drugs worsen the immunity and blood lipids, although they are frequently recommended in the literature. The difference between a patient suffering from depression and PTSD patient is that immune suppression in PTSD is substantially more pronounced than in simple depression patients[44].

It is common knowledge that patients suffering from severe mental diseases suffer from high pregnenolone, DHEA, or DHEA-S plasma levels. It is part of the nature of this invention that besides increase in hormones in the upper part of sexual hormone biosynthesis, are accompanied by low Testosterone, androstenediol, cortisone, estradiol plasma values. It is a major finding of the present invention that any improvement of severe mental diseases is characterized by a decrease of DHEA-S, DHEA, and pregnenolone and an increase of hormones in the lower part of hormone biosynthesis. Females who were raped as girls and suffer from PTSD, show increased DHEA-plasma values as well. PTSD patients share with patients suffering from schizophrenia or Bipolar Disorder, increased DHEA, or DHEA-S, and pregnenolone, plasma values.

It is a major claim of this present invention that special isoflavone containing soy formulations, fermented soy, red clover, alfalfa, and other isoflavone sources, either by natural or synthetic origin is able to reduce DHEA and pregnenolone, steroid hormones, which are synthesized close to cholesterol, whereas hormones at the lower part of biosynthesis are increased, like androstenediol, androstanediol, testosterone, etc. It has been shown in soldiers, that stress increases DHEA. Clearly hormone biosynthesis may vary in severe mental disease.

Too high or too low cortisol concentration, measured in urine, saliva or by blood sampling, are key biomarker in classic depression diagnosis. It is not known that androstenediol, the metabolite of DHEA, as well as androstanediol the metabolite of Dihydrotestosterone are key molecules of immunity and inflammation, and are also involved in anxiety. They are 100 times more potent in mediating immunity compared to DHEA. Any decline in “androstenes” have detrimental effects on immunity and inflammation and anxiety. It has been shown in elephants that changes of androstenediol bioavailability is related to stress. Mouse and rat models also showed that decline of androstenediol in hypothalamus and hippocampus causes stress.

One may argue an oral replacement of androstenediol would improver symptoms in patients suffering from PTSD. However hormone replacement would not increase hormone levels in cells, because it would be peripherally metabolized and would not reach nerve cells. Therefore it is a major finding of this patent that androstenes are increased intracellularly by increasing local metabolism from DHEA.

This disclosure claims that a restoration of normal bio-synthesis in the upper and the lower part of the hormone cascade is a key in reducing severe mental disease. It is state of the art and common knowledge that many steroids are produced by the testes, the ovaries, and the adrenals. The two most important are testosterone and estradiol. These compounds are under tight biosynthetic control, with short and long negative feedback loops that regulate the secretion of follicle stimulating hormone (FSH) and luteinizing hormone (LH) by the pituitary and gonadotropin releasing hormone (GnRH) by the hypothalamus. Low levels of circulating sex hormone reduce feedback inhibition on GnRH synthesis (the long loop), leading to elevated FSH and LH. The latter peptide hormones bind to gonadal tissue and stimulate P450ssc activity, resulting in sex hormone production via cAMP and PKA mediated pathways. The roles of cAMP and PKA in gonadal tissue are the same as that described for glucocorticoid production in the adrenals, but in this case adenylate cyclase activation is coupled to the binding of LH to plasma membrane receptors.

The biosynthetic pathway to sex hormones in male and female gonadal tissue includes the production of the androgens, androstenedione and dehydroepiandrosterone (DHEA). Testes and ovaries contain an additional enzyme, a 17β-hydroxysteroid dehydrogenase, that enables androgens to be converted to testosterone. In males, LH binds to Leydig cells, stimulating production of the principal Leydig cell hormone, testosterone. Testosterone is secreted to the plasma and also carried to Sertoli cells by androgen binding protein (ABP). In Sertoli cells the Δ4 double bond of testosterone is reduced, producing dihydrotestosterone. Testosterone and dihydrotestosterone are carried in the plasma, and delivered to target tissue, by a specific gonadal-steroid binding globulin (GBG). In a number of target tissues, testosterone can be converted to dihydrotestosterone (DHT). DHT is the most potent of the male steroid hormones, with an activity that is 10 times that of testosterone. Because of its relatively lower potency, testosterone is sometimes considered to be a pro-drug.

FIG. 17: Biosynthetic pathways of steroidal hormones. It is common knowledge that hormones close to cholesterol biosynthesis, which are synthesized in the adrenal glands, are increased in severe mental diseases (bipolar disorders, schizophrenia).

It is a major claim of the invention that special isoflavone containing soy formulations, fermented soy, red clover, alfalfa, and other isoflavone sources, either by natural or synthetic originis restoring normal biosynthesis of hormones. This was clinically determined in soldiers, which suffered from PTSD: Fermented soy did reduce gonadal hormones synthesized from cholesterol, whereas hormones at the lower part of biosynthesis are increased (FIG. 17).

It is a major claim of the present invention that PTSD patients suffer from high DHEA-S and pregnenolone plasma concentrations, a feature similar to Bipolar

### Disorders as Well as Schizophrenia.

It is a major claim of the current invention that it was found in a clinical trial, which will be outlines in the following section, that special isoflavone containing soy formulations, fermented soy, red clover, alfalfa, and other isoflavone sources, either by natural or synthetic origin are capable to modify selectively the correct cytokines and cause
1) balance mental suffering of PTSD patients like emotional numbness, anxiety, stress, sleeping disturbances, and in the long run depression.
2) reduce aggressive behavior, which frequently causes murder, or in the long term suicidal behavior.
3) Reduce completely impairment of immunity, like suffering from herpes infections, suffering from flu like symptoms, airway diseases like lung infections, and gastritis, which is caused by heliobacter pylori.
4) Reduce inflammation of the lung, gastritis, joints, and headaches.
5) Can substantially reduce drug substance abuse of alcohol, nicotine, cocaine and other similar dependencies developed by PTSD patients

Endogenous opiates (endorphins) are actively produced in the central nervous system (CNS) in response to stress. Endorphins represent one of the primarily major inhibitory neurotransmitter systems, inhibiting the release of other neurotransmitters, both in the CNS and in the peripheral organs. Endorphins can inhibit the neural transmission of sensory information in the spinal cord. Endorphins have been strongly implicated in the experimental paradigm of stress-induced analgesia. Conditioned stress induced analgesia is believed to be specifically endorphin dependent.

The limbic system of the CNS is where emotions, motivations and interests are processed and modulated. It is a region densely populated with opiate receptors. In PTSD endorphins are postulated to shut down the processing of emotional experiences and motivational systems, which leads to numbness and loss of interest. It is of interest that cytokines from the systemic blood circulation can enter the brain in the hippocampus and hypothalamus, as well steroids like androstenediol are produced locally in these areas of the brain. It is also an important claim of this current invention that local cytokine-release can be balanced in the brain by isoflavones consumed in the right doses as described herein. This is achieved by modifying hormone balances, and enzyme modifications in the brain.

Substance abuse is particularly high in PTSD patients, because they seem to counterbalance missing endorphins to reduce their emotional numbness. The relationship between substance abuse and numbness is so pronounced that some clinicians indicate that many cocaine abusers show signs of PTSD. It is our observation that special isoflavone containing soy formulations, fermented soy, red clover, alfalfa, and other isoflavone sources, either by natural or synthetic origin reduces not only numbness, but also substance abuse. It is a major claim of this patent that substance abuse is reduced by consumption of fermented soy.

Patients with severe mental illnesses, such as schizophrenia and bipolar disorder have an increased prevalence of metabolic syndrome and its components, risk factors for cardiovascular disease and type 2 diabetes. Although the prevalence of obesity and other risk factors such as hyperglycemia are increasing in the general population, patients with major mental illnesses have an increased prevalence of overweight and obesity, hyperglycemia, dyslipidemia, hypertension, and smoking, and substantially greater mortality, compared with the general population. Persons with major mental disorders lose 25 to 30 years of potential life in comparison with the general population, primarily due to premature cardiovascular mortality. The causes of increased cardio metabolic risk in this population can include no disease-related factors such as poverty and reduced access to medical care, as well as adverse metabolic side effects associated with psychotropic medications, such as antipsychotic drugs. Individual antipsychotic medications are associated with well-defined risks of weight gain and related risks for adverse changes in glucose and lipid metabolism. Based on the medical risk profile of persons with major mental illnesses, and the evidence that certain medications can contribute to increased risk, screening and regular monitoring of metabolic parameters such as weight (body mass index), waist circumference, plasma glucose and lipids, and blood pressure are recommended to manage risk in this population. Treatment decisions should incorporate information about medical risk factors in general and cardio metabolic risk in particular. In addition to the implications for individual clinicians, the problem of disparity in meeting healthcare needs for persons with mental illness in comparison with the general population has become an important public policy concern, with recent recommendations from the National Association of State Mental Health.

It is a major claim of this here outlined invention that a reduction of metabolic disorder in PTSD patients can be accomplished with fermented soy.

It is an important discovery of the present invention that PTSD patients suffer from increased MMP 3 and MMP 9, which was not known prior to this invention. MMP 9 increase in the endothelium of arteries and is related to cancer as well as to metabolic disorders, particularly stroke and heart infarction. It is a claim of this invention that fermented soy can reverse the increased MMP 3 and MMP 9 in PTSD patients[45,46].

Metabolic syndrome and MMP 9 gene expression are related. Inflammation and matrix degradation are the hallmarks of high-risk atherosclerosis that leads to myocardial infarction and stroke. Toll-like receptors (TLRs), key players in innate immunity, are up regulated in atherosclerotic lesions, but their functional role in human atherosclerosis is unknown. Several Matrix metalloproteinases, particularly 3 and 9, are increased in arteriosclerosis and are correlated to heart infarction and stroke risk.

For severe mental diseases like Bipolar Disorders and Schizophrenia, that member of the extracellular proteolytic system, composed of matrix metalloproteinases (MMPs) and their endogenous tissue inhibitors (TIMPs) have been shown to increase (increased MMP-9 levels, and, to a lesser extent, decreased MMP-2 levels in depression; increased TIMP-1 levels and MMP-9 levels in schizophrenia). MMPs display a key role in the central nervous system as well. They are able to process several proteins crucial for synaptogenesis, synaptic plasticity, and long-term potentiation (LTP). MMP-9 was specifically shown to regulate synaptic plasticity in the hippocampus by gain- and loss-of-function studies on LTP in vitro.

Experimental evidence supports the hypothesis that the MMP/TIMP ratio modulates neuronal plasticity in learning and memory processes. TIMP-1, which binds to MMP-9 and regulate its activity, was indeed shown to be able to prevent MMP-9-dependent late LTP in the rat medial PFC. MMPs and TIMPs have been also investigated as potential markers for dementia, resulting with the identification of altered plasma levels of MMP-9 and TIMP-1 in Alzheimer's Disease and vascular dementia, respectively.

MMPs have many other properties, including the ability to modulate cytokines and growth factors (such as TNF-alpha and BDNF (Brain-derived neurotrophic factor) among others) by processing their proforms into active forms. Interestingly, similar to what we have found in our case, patients with metabolic syndrome also display increased circulating concentrations of pro-MMP-9, MMP-8, and TIMP-1, which were associated with increased concentrations of proinflammatory mediators and adhesion molecules. Of note, our supplemental data show a positive association of TIMP-1 levels with age in MDD patients and controls, which is not detected in the schizophrenia group which has elevated levels in spite of a lower mean age with respect to controls.

It has been found for the first time and it is therefore part of this disclosure that MMP 3 and MMP 9 decrease in severe mental diseases like Schizophrenia, Bipolar Disease, PTSD and likewise is correlated to improvement in wellbeing and in metabolic disorders[47].

It is the main objective of the present invention to provide a new medication to treat simultaneously severe mental disorders like posttraumatic stress disorders (PTSD), schizophrenia, Bipolar Disease, and its accompanying metabolic syndrome, as well as immunity and inflammation disturbances. Severe mental disease causes complete loss of control of life and leads to complete withdrawal from society but also causes in Bipolardisease and in PTSD, aggression and/or auto-aggression behavior leading to murder and/or suicide. Patients suffering from severe mental disorders have a reduced life expectancy due to accompanying metabolic disease, leading to diabetes, heart infarction and stroke. Special biotechnology derived special isoflavone containing soy formulations, fermented soy, red clover, alfalfa, and other isoflavone sources, either by natural or synthetic origin reduces the increased gonadal hormones of PTSD patients, Schizophrenia, and bipolar disorders synthesized in the upper part of biosynthesis, DHEA and pregnenlone, at the same time increases reduced hormones at the lower part of biosynthesis like androstenediol, cortisone, and testosterone. This balances Th1/Th2 cytokines like TNF-alpha and IL-1, which regulate immunity and inflammation. The fermented soy formulation not only reduces emotional numbness, stress and aggression, but also normalizes arteriosclerotic blood profiles, which will lead to normal life expectancy. Fermented soy improves gene expressions, which link a) mental signs, b) cytokine release and c) arteriosclerosis like MMP 9 (matrix metalloproteinase 9). Formation of fermented soy formulation is outlined and defined.

### Example 1

### Clinical Study with Veterans Suffering from PTSD

PTSD is defined as severe disturbance with any kind of stress and with social retreat after a severe traumatic event. Clinically, PTSD is accompanied by sever signs of aggression/auto-aggression threatening others and the patient himself. PTSD patients suffer from signs of “metabolic diseases” and diabetes, causing a substantial reduction of life expectancy.

This invention claims that formulations composed of special isoflavone containing soy formulations, fermented soy, red clover, alfalfa, and other isoflavone sources, either by natural or synthetic origin with special ingredients, are capable to balance hormone levels, particularly decrease elevated DHEA and pregnenolone, and increase hormones like androstenes, testosterone, estradiol. As a consequence a reduction of the disturbances of immunity and inflammation takes place. A complete remission of PTSD Symptoms, as well as its accompanying metabolic diseases is achieved.

Preferably, fermented soy is administered orally in an initial dosage of higher than 300 mg of isoflavones a day, better 500 mg of isoflavones/day or even higher for about two months. The dosage can be decreased until the patient has achieved a numb-free state. To get this much isoflavones, one can consume 80 ml of FSWW08 brand fermented soy product (for 300 mg per day) or 140 ml (for 500 mg per day). 100 ml of FSWW08 brand fermented soy product provides about 400 mg of isoflavones and about 600 CCl of protease inhibitors (about 400 CCl of Bowman Birk Inhibitory Factor).

Symptoms related to bipolar disorders, schizophrenia, or PTSD, i.e., conditions exhibited by an individual which are a by-product of the emotional numbness, such as anxiety, lack of empathy, mental confusion, amnesia, loss of interest and compulsive sensation seeking behavior are reduced by the method of the invention. The emotional numbness itself may be associated with one or more psychopathologic conditions, such as PTSD, depression, hypochondria, anxiety, a psychosomatic disorder or negative symptoms of schizophrenia, or the emotional numbness may be associated with one or more physical insults to the central nervous system such as a closed head injury or a cerebral vascular accident.

Patients are showing signs of aggression against others, however are also in danger of committing suicide. Substance abuse is very high in PTSD patients. PTSD will automatically develop into diabetes, hypertension, and what is called metabolic diseases. PTSD patients are suffering from social isolation.

It is a claim of this invention and this patent that under therapy with special isoflavone containing soy formulations, fermented soy, red clover, alfalfa, and other isoflavone sources, either by natural or synthetic origin, starting within the first week, PTSD patients will lose their emotional numbness, show signs of integration into social activities and particularly a reduced substance abuse behavior. Aggressive behavior against others is completely abolished as well any signs of stress. Accompanying diseases like immune disturbances, and inflammations are completely eliminated. Appearance is improved that means PTSD patients show increased personal hygiene, which is caused by increased self-esteem, but also improved skin appearance, which is caused by fermented soy.

Patients suffering from severe mental diseases like PTSD, schizophrenia, bipolar disorders show in their circulating blood increased IFN-γ, a reduced TNF-α value, an increase of testosterone, increase of DHT, and normalize cortisol. Triglyceride as well as cholesterol value is normalized, which is accompanied by a lowering of hypertension.

Haelan Research Foundation, Woodinville Wash., USA, supplied packaged bottles of fermented soy product (FSWW08) The volume of the bottles was about 225 ml/bottle.

In general, the present invention relates to the use of fermented soy, for the treatment of mental disturbances of PTSD and other psychopathologic conditions and organic symptoms of PTSD, particularly immune disturbances and inflammation conditions.

It has been discovered and is a major claim of this patent that when PTSD is healed, in accordance with the method of the here outlined invention, certain secondary characteristics or symptoms related to PTSD begin to disappear or at least begin to lessen significantly. In other words, when PTSD is successfully treated, then other conditions or symptoms associated with it are also treated. Some of these conditions or symptoms of PTSD include, but are not limited to PTSD, empathy, mental confusion, amnesia, loss of interest, and compulsive sensation seeking behavior. PTSD includes other psychopathologic conditions such as depression, hypochondria, anxiety, a psychosomatic disorder and negative symptoms of schizophrenia. Moreover, PTSD may be associated with any of various physical insults to the central nervous system, such as, but not limited to, closed head injuries and cerebral vascular accidents.

This compound is particularly effective by the oral route and is also effective when administered rectally, although any suitable route may be employed. Fermented soy is preferably combined (mixed) with a pharmaceutically acceptable inert carrier for easy ingestion. Suitable inert carriers include, but are not limited to, water, milk optionally with sugar and/or starch, natural and synthetic fruit juices, such as orange juice, grapefruit juice, grape juice, pineapple juice, lemon juice and prune juice, and sweetened beverages such, for instance, as flavored water with or without carbonation. Sweetener can be artificial sweetener as well as stevia. If the compound is to be administered orally, one of the above carriers is desirable. If the compound is to be administered parenterally, distilled water is a desirable carrier.

The compound also may be administered rectally by incorporation in a standard suppository or oral form. Other routes of administration and suitable pharmaceutically acceptable carriers therefore will be apparent to one skilled in the art.

The method of the invention is recommended for any individual who has PTSD and/or other psychopathologic conditions noted above, in whom numbness is persistent and interferes with that person's ability to enjoy life's activities and to relate to significant others or is depressed. During that period, the individual should have shown an ability to consistently relate to significant others and to experience feelings rather than numbness.

Dosage changes may be instituted relatively quickly as long as the patient is followed carefully on a weekly basis.

Preferably, in the method of the invention, the patient is started on fermented soy at about 500 mg isoflavones a day. Some symptoms in some individuals may persist for a longer period of time before they reverse. Normally PTSD patients report significant improvement after 7 days.

The invention will be more fully understood by reference to the following examples: Post-traumatic stress disorder (commonly referred to by its acronym, PTSD) is a prevalent and disabling disorder. PTSD is a severe anxiety disorder that can develop after exposure to any event, which results in psychological trauma.[1,48]. This event may involve the threat of death to oneself or to someone else, or to one's own or someone else's physical, sexual, or psychological integrity, overwhelming the individual's psychological defenses[1,48]. By definition prior psychological trauma plays a causal role in the disorder, and psychotherapy is a widely accepted intervention [49]. Nevertheless there is growing evidence that PTSD is characterized by specific physical dysfunctions, and this has contributed to a growing interest in the use of medication in its treatment, although no success was achieved, yet[50]. It is state of the art and known to professionals in the area of treating those who suffer from the disease, that PTSD is frequently accompanied by other mental diseases like depression [51] and may accompany many life-threatening diseases like e.g. cancer[52]. PTSD will ultimately lead to many other health related complications like cardiac dysfunction[53,54], virus infections[55] just to name a few.

The present invention describes a new method where both PTSD and its accompanying diseases are treated. The invention was clinically tested in severely traumatized ISAF-NATO-soldiers, only recently returning from Afghanistan suffering from PTSD, not longer than a year (8-12 month). When they left for duty, all were mentally and physically healthy, documented by extensive medical records within the military. After developing symptoms of PTSD, all soldiers suffered additionally from two types of diseases: Firstly, disturbances of immunity and secondly, high degree of inflammations. Disturbances of immunity caused high rates of herpes infections as well as constant suffering from flue. Generally, PTSD patients suffer an 11 times higher risk of developing Hepatitis B and C infections[57]. Also, PTSD soldiers suffered from inflammation of the stomach, joints [58], lung [59,60], and complain frequently of headaches[61]. Since these soldiers did not suffer from classical depression or burnout like September 11 World trade center victims[62], although they all suffered from sleeping disturbances, we hypothesized that PTSD suffer from Th1/Th2-cytokine balances. Increase of Th2-cytokines causes inflammation, whereas decrease of Th1-cytokines causes infections. Most of all, these cytokine can cross the blood brain barrier. It has been reported that brains of PTSD patients in Computer Tomography resemble those of suffering from brain injuries[63].

In vivo, the androstenes increase the levels of the Th1 cytokines such as IL-2, IL-3, and IFN-γ and increase immunity (FIG. 4)[65]. Similar to hydrocortisone, they suppress inflammation, but without immune suppression (FIG. 3), and have a role in the maintenance of the Th1/Th2 balance and immune homeostasis. They are also capable to decrease Th2 cytokines, which are capable to increase inflammation (FIG. 9,14).

FIG. 18: Structure formulas of androstenes and soy isoflavones. Androstenediol is metabolized from DHEA.

Androstenes also reduce stress [66,69], which is the major clinical sign of PTSD. Androstenes are metabolized from their precursor testosterone or DHEA in the brain (FIG. 11)[68,70]. Since it is not possible to estimate from peripheral blood androstenes levels those in the brain, replacement via peripheral routs of administration may be difficult to achieve. We therefore were looking into another possibility to restore Th1/Th2-balance via molecules similar to Androstenes. Fermented soy, which contains molecules like isoflavones did show in children with reduced immunity, a decrease of Th1-cytokines and an increase of Th2-cytokines and helped to reduce life-threatening allergy[71]. Soy isoflavones are similar to andostenes structurally and pharmacologically (FIG. 18).

Fermented soy (SOY-IQ) was given daily to soldiers suffering from PTSD in an outpatient setting to investigate over two month treatment on symptoms of PTSD. Clinical effects were documented by standard questionnaires. Cytokine- and hormone-concentration were analyzed in the blood, as well as blood chemistry was determined. Clinical results were corroborated by gene expression studies of investigating monocytes of soldiers suffering from PTSD, before and after consuming soy.

FIG. 19: Metabolism of DHEA is inhibited in women suffering from PTSD as a consequence of sexual abuse during childhood.

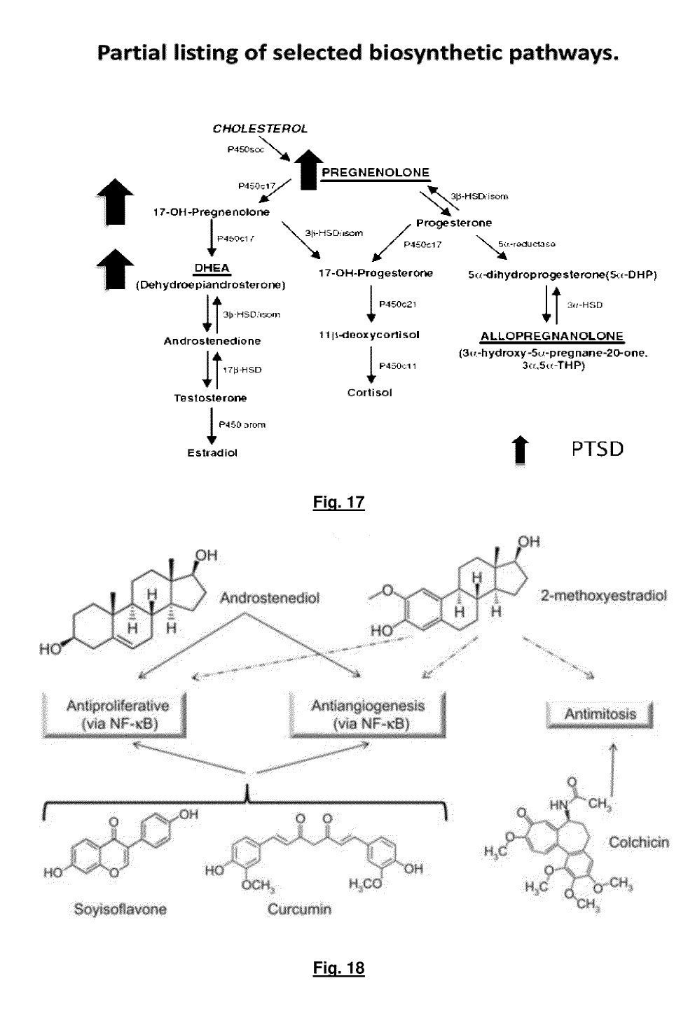

FIG. 20: Schematic depiction of relationship between andostenes and immunity and inflammation.

Prior to our own investigation, all enlisted soldiers were diagnosed by Military physicians and it was stated that they were suffering from post-traumatic stress (PTSD) by the ICD-10 code falling into the F43.1 category defining PTSD. To be enlisted in this study the major inclusion criteria was that they experienced themselves a life-threatening event prior to the disease. Also, participants needed to be in the military. Raw characteristics of the soldiers are depicted in Table 4. Based on these requirements no exclusion criteria, besides that all soldiers were able to follow advice by the study physician. We were interested to recruit in our study soldiers, who did not suffer for more than a year from PTSD. Although co-morbidity was not pronounced, all soldiers abused alcohol consumption and were nicotine abusers. Due to low self-esteem, soldiers did not take care of themselves in proper clothes or personal hygiene. Blood chemistry did reveal high cholesterol and triglyceride values (FIG. 25). Soldiers were stressed, so that lengthy clinical investigations had to be interrupted frequently. Although almost all soldiers were suffering from breathing problems, documented investigation (like peak flow measurements) could not be conducted because stress levels were too high.

Each soldier did receive a complete full medical exam, lasting several hours. This included, lung functions, exam of the head, throat, skin, and testing nerve reflexes, palpation of the skin, lymph system. Checking for joint pain, muscle pain, etc. Examination included checking medical history including medical history of their parents and first grade relatives. Mental disease within the family was not specifically checked, but revealed no predisposition in any of the soldiers.

Prior to our study, soldiers suffered 8-12 month from PTSD, not long enough to be effected by co-morbidity of other mental diseases like depression. Although some clinical parameters indicated that they would soon develop severe metabolic syndrome or diabetes. We did provide a PTSD-questionnaire to the patients, who were developed by soldiers suffering from PTSD. We additionally did provide a questionnaire, which is designed to document effects and side effects on medication on patients in clinical trials EORTC QLQ-C30 (version 3).

All patients signed a document that they conducted the study with informed consent. This included that they did receive prior to signing the document information that fermented soy (SOY-1a) was not approved for this indication, however purely experimental and no health claim has been issued yet.

A local ethics committee, including the head of the local Military Hospital, did approve the study and it was expressed that the Convention of Helsinki in its current form was followed. All soldiers were informed that they could leave the trial at any time without expressing reason. No copies of the records were and will be taken out of the Military Hospital. All information will be written in a way that anonymity for each soldier is granted.

Blood samples for further processing were shipped to a facility in Germany. Handling, flight and storage was performed accordingly a procedure developed by the laboratory.

Study medication was received from Haelan Research Foundation, Inc. Woodinville, Wash. USA. Shipping, storage and handling protocol followed a procedure developed by Haelan Research Foundation Inc. in the USA. The study medication has the approval of a food for special medical needs in the EU and the USA. Fermented soy was provided by:
Haelan Reserch Foundation Inc
17825 149th Ave NE
Woodinville, Wash.
USA
Phone: 425-269-7798
Fax: 425-482-0522

TABLE 4
Selected clinical parameter of enlisted soldiers

| | At the beginning of the | At the end of |
|---|---|---|
| | study | the study |
| Age | 32 (from 26 to 38) | — |
| Height | 1.79 m | 1.79 |
| Weight | 82 kg (78-91) | 85 (79-94) |
| BMI | | |
| Number of Patients | 10 | 9 |
| enlisted in the study | | |
| ISAF - NATO-Soldiers | 10 out of 10 | 10 out of 10 |
| stationed in Afghanistan | | |
| Soldier did experienced | 10 out of 10 | 10 out of 10 |
| Life or near Life | | |
| threatening Situation | | |
| When did PTSD develop | Between 3 month to 1 | — |
| after the life threatening | year | |
| incident? | | |
| Returning from | 1 to 1½ year | — |
| Afghanistan | | |
| prior to enlistment in the | | |
| study | | |
| PTSD diagnosed prior | 8 month to 12 month | — |
| enlistment to the study | | |
| CRP | Normal | Normal |
| Sleeping disturbances | 10 out of 10 | 0 out of 10 |
| Feeling numb | 10 out of 10 | 0 out of 10 |
| Feeling stress | 10 out of 10 | 0 out of 10 |
| Lung infection/short breath | 3 out of 10 | 0 out of 10 |
| Stomachache | 6/10 | 0 out of 10 |
| Herpes infection | 6 out of 10 | 0 out of 10 |
| Flue-like symptoms | | |
| Other virus infections | | |
| Substance Abuse | 10 out of 10 | 10 out of 10 |
| | alcohol, tobacco | Alcohol, tobacco |

Characteristics of soldiers in the clinical study: Properties were evaluated before and after 3-month treatment with fermented soy (SOY-IQ).

All soldiers reported an improvement in wellbeing in the first 2 weeks of consumption of fermented soy. Emotional numbness did disappear, as well as an improvement in sleeping habits, a dramatic reduction in stress, anxiety. All soldiers were cachectic prior to the study, although their body weights were in the accepted medical limits. Eating habits did return to normal levels.

Plasma serum specimens were obtained from patients from a Hospital treating NATO-ISAF soldiers suffering from PTSD in December 2009 to April 2010. The samples were sent per overnight express to BIOFOCUS in Germany. Samples were processed within 12 hours of blood draw. The blood was allowed to clot at room temperature for 15 minutes and was stored at 4° C. until centrifugation at 3500 rpm for 15 minutes. After processing, all samples were aliquoted and stored immediately at −80° C. We and others have observed that sera stored at −70° C. and lower for 9 months or more showed no significant differences in prostate specific antigen (PSA) levels compared with fresh samples. Similar observations have been made regarding the stability of cytokine serum levels. The specimen aliquots had not been thawed prior to this study. TNF-alpha, IL-1 beta, IL-6, and IFN-gamma were determined with Immulite assays (Diagnostic Products Corporation, Los Angeles, Calif.) and were used to determine serum cytokine levels respectively.

All assays were performed according to the manufacturer's instructions. Intensive medical check was performed before and after 3-month consumption of fermented soy. The soldiers were advised to consume four fluid ounces, a half bottle, of fermented soy daily in the morning.

Although specific PTSD questionnaires are available, we used a questionnaire commonly used for patients who receive medication for drug treatment.

FIG. 21: A complete reduction of stress of 9 NATO-ISAF Soldiers suffering from PTSD, According the EORTC QLQ-C30 (version 3) questionnaire.

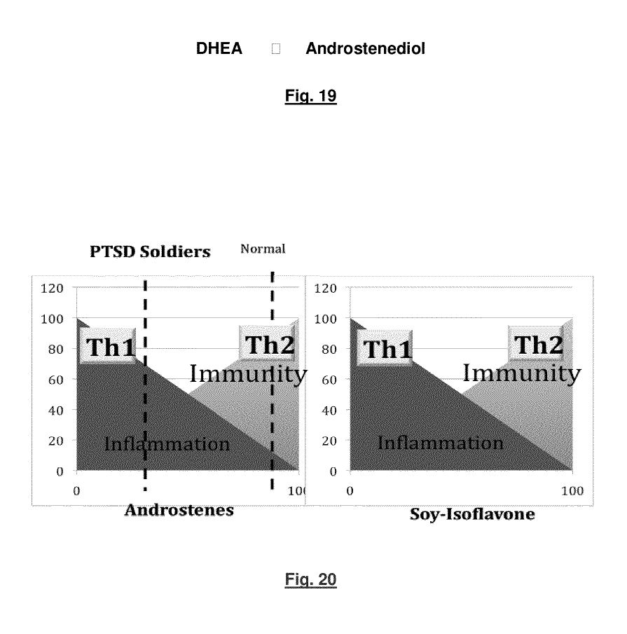

FIG. 22: Influence of consumption of fermented soy on anxiety in NATO-ISAF-soldier suffering from PTSD, According the EORTC QLQ-C30 (version 3) questionnaire.

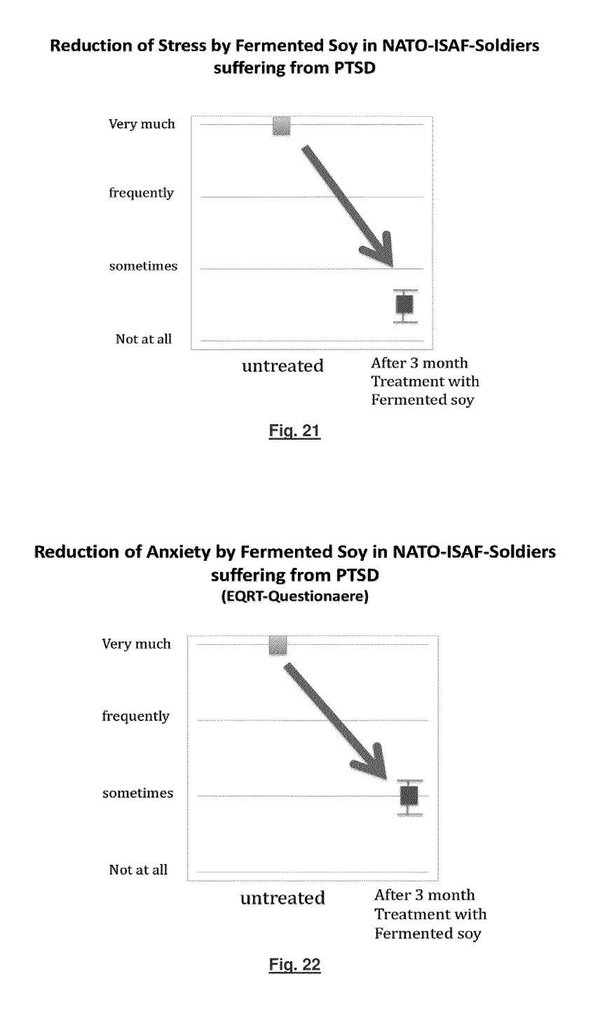

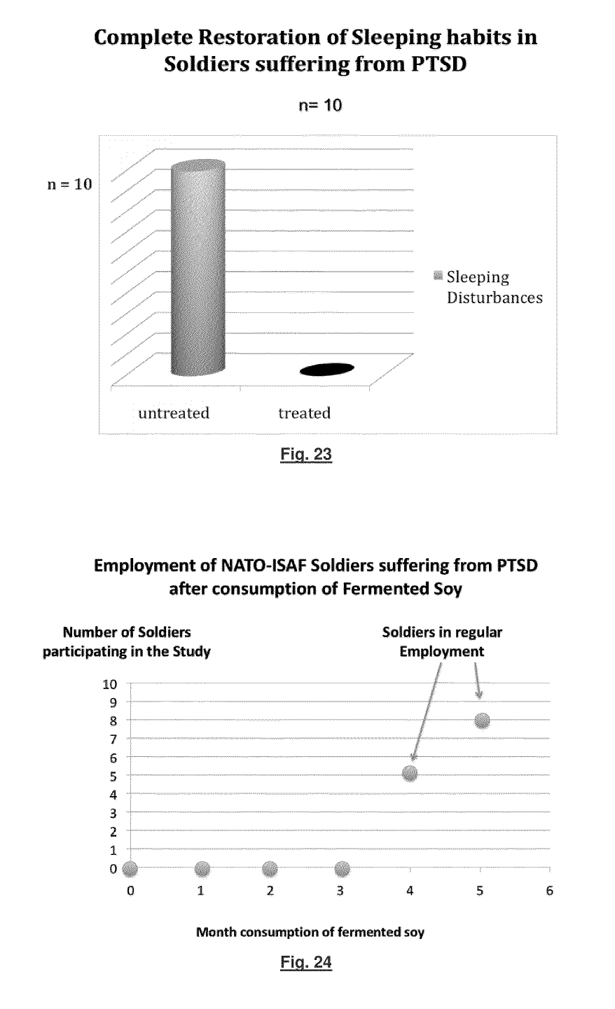

FIG. 23: Influence of fermented soy on sleeping habits of NATO-ISAF soldiers suffering from PTSD According the EORTC QLQ-C30 (version 3) questionnaire.

FIG. 24: Employment of NATO-ISAF soldiers and consumption of fermented soy (SOY-IQ). Clinical trial did last 3 month. Soldiers did continue consumption of fermented soy.

As can be seen in FIG. 21 stress was completely reduced in all soldiers suffering from PTSD. Anxiety disturbances are also completely reduced (FIG. 22). Prior to the study all soldiers suffered from sleeping disorders. Sleeping habits were completely restored after consumption of fermented soy (FIG. 23). Immune disturbances and inflammation were completely reduced by fermented soy (Tab. 4,5).

TABLE 5
Influence of consumption of fermented soy (SOY-IQ) on
immunity and inflammation disturbances of NATO-ISAF
soldiers suffering from PTSD.
Reduction of Inflammatory or Immunty Disturbances in
NATO-ISAF-Soldiers suffering form PTSD

| | | After 3 month consumption |
|---|---|---|
| | Untreated | of fermented soy |
| Inflammatory Diseases | | |
| Gastritis | 6 of 10 | 0 |
| Lung inflammation | 4 of 10 | 0 |
| Headache | 5 of 10 | 0 |
| Infectious Disease | | |
| herpes labialis | 5 of 10 | 0 |
| sore throat | 4 of 10 | 0 |
| Flue like symptoms | 6 of 10 | 0 |

FIG. 25: Improvement of blood compounds related to metabolic syndrome in NATO-ISAF-soldiers suffering from PTSD, by fermented soy.

Most importantly, the majority of soldiers did return to employment after the study (FIG. 24). They did continue consumption of fermented soy up to 6 month. All soldiers quit the army and returned to a regular job outside the army. As can be seen in FIG. 8, and is a major claim of this invention, the improvements in clinical parameters were accompanied by a substantial improvement in blood parameters, which are frequently related to metabolic syndrome (FIG. 25). The most improvement was seen in triglycerides, as well as in cholesterol, homocystein (FIG. 25). An increase in HDL as well as a decrease in LDL-cholesterol was seen (not shown here).

FIG. 26: Influence of consumption of fermented soy on DHEA-S blood values in NATO-ISAF-soldiers suffering from PTSD. Left graph individual values, right graph average values and SD.

It is a major claim of this invention that DHEA, 17-OH-pregnenolone, as well as pregnenolone (not depicted here) were decreased in NATO-ISAF soldiers suffering from PTSD, when they consumed fermented soy (FIG. 26,27).

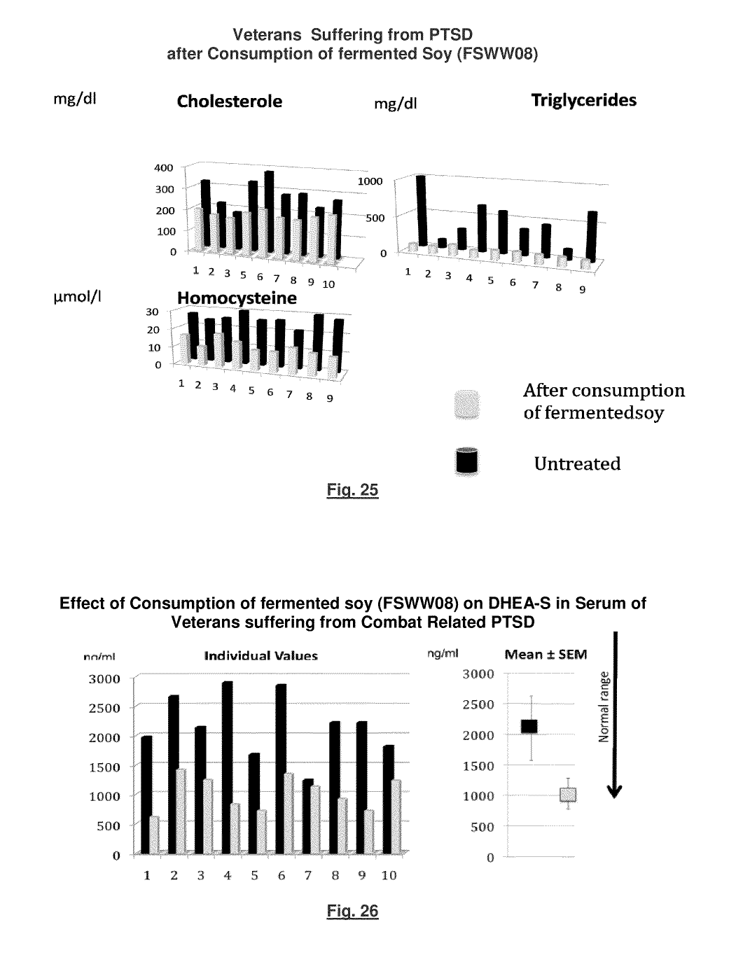

FIG. 27: Reduction of 17-OH-Pregnenolone in serum of NATO-ISAF soldiers suffering from PTSD under consumption of fermented soy. Individual and mean and SEM are depicted. Grey range summarized the optimal range.

It is a major claim of this invention that in contrast to steroids synthesized in the upper part of the biosynthesis, most steroids like testosterone, androstenediol are reduced and increased by consumption of fermented soy (FIG. 13).

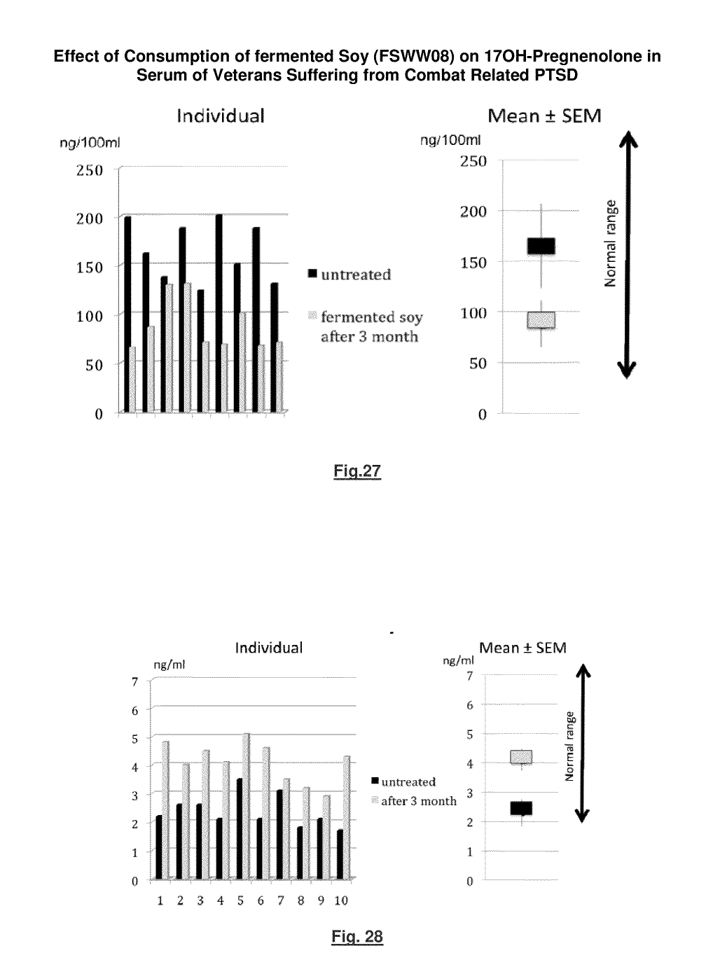

FIG. 28: Increase of testosterone by fermented soy in PTSD patients. Other hormones increased are cortisone, androstenediol, and estradiol.

TABLE 6
Summary of several cytokines in 10 NATO-ISAF soldiers
suffering from PTSD after consumption of fermented soy.

| | | After 3 month | |
|---|---|---|---|
| | | consumption of | Recommended |
| Cytokine | Untreated | fermented soy | range |
| TNF-alpha (pg/ml) | 13.5 ± 0.4 | 9.0 ± 1.4 | <9.5 |
| IL-1beta (pg/ml) | 7.0 ± 0.5 | 4.5 ± 1.8 | <5 |
| IL-6 (pg/ml) | 2.3 ± 0.3 | 2.2 ± 0.5 | <8.5 |
| IFN-gamma (pg/ml) | 4.4 ± 1.0 | 7.0 ± 1.5 | <5 |

Fermented soy modulates selective cytokines in plasma (Table 6). TNF-alpha is related to inflammation as well as cachexia. IL-1 beta is a cytokine related to cachexia. IL- is almost always increased in depression. PTSD patients did suffer from anxiety and stress disorders but not from depression. All soldiers did gain weight (FIG. 29), what corroborates the decrease of IL-1beta.

FIG. 29: Individual Increase of body weight in cachectic NATO-ISAF soldiers suffering from PTSD after 3-month consumption of fermented soy.

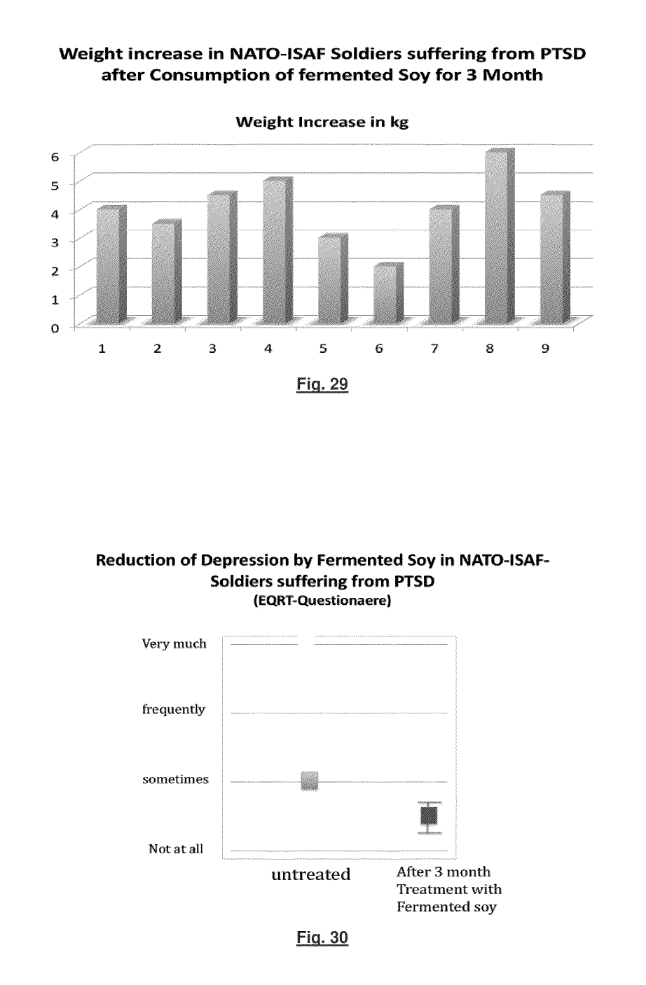

IL-6 was not altered, what corroborates the observation that PTSD patients involved in this study were not suffering from depression (FIG. 30). Improved cytokines may be also related to a reduction of inflammation and the infections, particular viral infections depicted in Tab. 5.

FIG. 30: Effect of consumption of fermented soy on wellbeing in NATO-ISAF soldiers suffering from PTSD, according the EORTC QLQ-C30 (version 3) questionnaire.

FIG. 31: Summary of investigation of hormonal steroids in serum of PTSD patients. DHEA, pregnenolone are increased, whereas hormones at the lower end are decreased.

It is a major claim of this invention that fermented soy balances hormones in the serum, balances blood profiles related to stroke and heart infarction, as well as diabetes. Immune status is increased, so that less viral infection can occur, as well as inflammatory diseases. Investigating blood samples revealed, all soldiers suffered from high Th2-vakues TNF-alpha (Tab. 6) and low Th1 values (IFN-gamma). They also showed relatively low testosterone, cortisone plasma values. However, no increase of metabolism was detected, supporting the observation, that soldiers were not depressed, according to the classical definition of depression. All soldiers were cachectic, due to increased Th2 levels, particularly IL-1.

Within one month of treatment with fermented soy, all soldiers showed almost complete remission of all symptoms related to PTSD. All soldiers reported remission of emotional numbness.

Fermented soy balanced disturbances of Th1/Th2-balance, assuming that they are causing tremendous disturbances of inflammation and immunity in the brain.

Although cytokines were measured only in the periphery, cytokines cross the blood brain barrier and cause serious disturbances in immunity and inflammation [74].

This patent claims a complete remission of all mental as well as restoration of normal immune function, and blood lipids of PTSD patients.

Normally the decline of precursors of adroitness, testosterone and DHEA, are related to increase of inflammatory cytokines, causing age related inflammation. The metabolite of DHEA androstenediol and andostenetriol, increase the anti-immunity compared to DHEA in the ratio 1, 100, 1000 [65]. Therefore we conclude that the reduced immunity of PTSD patients is not a co-morbidity to PTSD, but our findings may explain, why conventional anti-depressants show no efficacy in the treatment of PTSD, because they do not modify Th1/Th2-cytokines over a wide range [75].

There is a direct correlation between Th1 and an indirect correlation of Th2-cytokines with androstenes in humans [76].

### Example 2

### The Case of the Schizophrenic Boy

A child was brought to us due to poor school performance in exams. The child was previously a brilliant student. He used to perform very well academically and always stood first in his class without any problems. The child was previously a brilliant student and always stood first in his class without any problem. When he was 11 yrs old, the parents moved to another part of Vienna and school changed. Since he changed previously from elementary school to High School. The new system was a much tougher and complex system of education.

All the problems of this child started thereafter. This change in education came as a big shock to him. He developed this idea that he would not be able to cope up with the new education. He had a constant anxiety that he has not studied at all and would surely fail in his exams. He felt that other students would go ahead of him. Every half an hour, he would call up his friends to ask how much portion they had completed.

He became very sensitive, irritable, anxious and fearful of everything. He developed fear of studies, of dark, of lonely places, of exams, of failure. He started developing delusions and hallucinations, like he would hear noises and voices from far off places. He had a feeling that there was something wrong with him or that somebody was chasing him.

His concentration level dropped down. He started forgetting whatever he had done. He developed a lot of confusion. He would read the same page again and again as if he had never understood what he had read. He did not feel like studying anymore.

He felt that he should leave the studies. He started praying that he should get some illness, which will prevent him from appearing for exams.

Sleep was disturbed. He would often get up from sleep or would not be able to sleep for hours together.

Sensitivity to noise increased. Even if the television was on four rooms away, he could hear it. He could hear the noises from 2 or 3 buildings away. He developed marked sighing and cried often.

It was quite evident from this case that the child could not bear the stress of the new advanced pattern of education and he was collapsing under this stress.

### Developmental Milestones:

Birth wt. 6½ pounds
Talking 11 months
Walking 1¼ year
Teething 7 months

### Diagnosis: Childhood Schizophrenia and Diagnostic Criteria, Characteristic Symptoms:

Two of these symptoms were present for a significant portion of time during a six month period or more:

### Delusions

### Hallucinations

### Social/Occupational Dysfunction:

For a significant portion of time, since the onset of the disturbance, the major areas of functioning of work and self-care are markedly below the level achieved.

### Duration:

Continuous signs of disturbance persist for at least 6 months or more.

This has been ruled out as a Schizoaffective and Mood Disorder Exclusion because: No Major Depressive, Manic or Mixed Episodes have occurred concurrently with the active-phase symptoms.

### Symptoms Considered:

Fear: Failure, of Mood, disposition: despairing, hopeless, discourage

### Fear: Dark

Fear: Alone, of being

### Delusions, Imaginations:

Walk: Someone walks behind
Concentration difficult [see comprehension thinking]:
Dullness, sluggishness difficulty of thinking and comprehending
Delusions: imaginations: Noise, hears
Work: Aversion to mental
Sensitive, oversensitive: Noise to:
Weeping, tearful mood: Tendency
Illusions, delusions, visions, etc., wrong, everything
Walking, delay in

The boy developed increased virus infection and he developed flue like symptoms.

### Compilation of Mental Symptoms:

### Apprehensive;

worse towards evening;

### Fears Loss Of Reason,

### Misfortune,

contagious diseases, constant flue like symptoms,
Forgetful, confused, low-spirited.
Anxiety with palpitation.

### Obstinacy:

Slight mental effort produces hot head.
Averse to work or exertion.

### Treatment of the Schizophrenic Boy

Half a bottle was given a day of Fermented soy (FSWW08)

After receiving FSWW08, The Child was coming up strongly. All the delusions were covered by Fermeneted soy (FSWW08) and the other two remedies before he came to our office did didn't cover them at all.

We confirm compliance taking the medicine through asking the mother.
Prescription: 15th Mar., 2008: Remedy: half a bottle FSWW08 as a consequence on

### 21th Mar., 2008

Depression less
Concentration better

Was able to study, was worried about his position

### 21st Mar., 2009

Further amelioration

Able to appear for the exams and no problems

### 15th Jun., 2008

Patient reports that he has stood 1st in the class.

### 28st Jun., 2008

Studying very well
Has stood first in all exams without any anxiety episodes.
No other complication or problems.

### SUMMARY

The most important improvement was the reduction of stress. The boy became focused again. Also constipation and a complete reduction of contagious diseases including flue like symptoms are reduced. All schizophrenic symptoms were relieved. The boy did drink the fermented soy solution (FSWW08) freely, because he knew that his performance in school improved due to the medication and did loose all stress like and anxiety symptoms.

### Example 3

### A Bipolar Pregnant Women, During Pregnancy and Postpartum

A 32 years old pregnant women came to our office reporting that she was bipolar and was under antidepressant and anti-psychotics treatment before she was not pregnant. She was pregnant the second time and showed similar maniac and depressive episodes as seen in the first pregnancy. She was concerned receiving no pharmaco-treatment, however was even more concerned that medication had negative effect on her unborn child. Pregnancy did slightly increase Bipolar Disorder in her.

She suffered tremedously form postpartum hypomania, but experienced severe depression. And there was a quick onset of depression immediately after delivery.

The following observations were made last time:
cortisol levels were decreased postpartum
Atypical features (DSM-IV criteria), racing of thoughts, and concomitant psychotic symptoms were seen.
Despite depression symptoms, she did show to antidepressants rapid response, loss of response.]

### Etiology of the Disease

Her mother suffered already from Bipolar Disease and depression, supporting the observation that a first degree relative frequently suffers form severe mental disease, too. She showed substantial co-morbidities like flue like symptoms, contagious diseases and showed tremendous stress, before, during and after pregnancy. After delivery, she suffered from sleeping disorders and constipation.

During her pregnancy the Bipolar disease did increase and she had no depressive episodes. After pregnancy she became very depressed and suffered also form anxiety. Cleary, it is diffcult to diagnose a women postpartum as Bipolar. Frequently they are diagnosed as depressive, which may be a comorbidity and not the main mental disease.

She was pregnant in the 5th month, when she attended our office.

For her, any medication during pregnancy and breast-feeding has to fulfill several criteria, especially that they have no negative effects on fetus and breast-feeding. Since fermented soy is given babies as a nutritional product for 10 years without showing any negative on born babies, it was concluded its negative effects on fetus and babies can be ignored.

General consideration are (Sharma. Can J Clin Pharmacol Vol 16 (1) Winter 2009):
Avoid antidepressants, if possible
For patients requiring medication during pregnancy, try using monotherapy at minimally effective doses
Consider its effectiveness in the prophylaxis of postpartum mood episodes before selecting a
drug to treat depression during pregnancy
Benefits of breastfeeding should be balanced against the deleterious effect of sleep deprivation in triggering mood episodes
Close monitoring of mood and sleep
Target postpartum sleep disruption with adjunctive treatment

We did give her a bottle a day of fermented soy (FSWW08), starting in the 5^{th} month of pregnancy. Maniac episodes disappeared after 10 days did not returns as long as she took FSWW08.

In the postpartum period she did not continue medication until day 21. The symptoms of depression and Bipolar syndrome did return and she was given fermented soy (FSWW08). She suffered especially form sleeping disorders, not only caused by the baby. Several steroidal hormones before and after application of FSWW08 and cytokines related to Bipolar depression were determined.

| | | 2 month after | |
|---|---|---|---|
| | | Delivery | |
| | Day 14 after | under | |
| | delivery | consumption of | |
| Hormone | Untreated | FSWW08 | Normal range |
| Cortisol (μg/dl) | 3.8 | 10.8 | 5.0-25.0 |
| 8.00 am | | | |
| DHEA (nmol/l) | 21.8 | 10.1 | 6.24-43.3 |
| Pregnenolone (ng/ml) | 3.8 | 2.5 | 0.38-3.5 |
| Estradiol pg/ml | 32 | 64 | 50-200 |
| TNF-alpha | 13.5 | 7.0 | <9.5 |
| (pg/ml) | | | |
| IL-1beta (pg/ml) | 7.0 | 4.5 | <5 |

Lipid markers

| | | | |
|---|---|---|---|
| Cholesterol (mmol/l) | 320 | 214 | <200 |
| Triglyceride | 348 | 151 | <150 |
| (mg/dl) | | | |
| HDL-cholesterol (mg/dl) | 51 | 89 | 40-60 |
| LDL-cholesterol mg/dl | 322 | 151 | <155 |
| TNF-alpha | 12.5 | 9.5 | <9.5 |
| (pg/ml) | | | |
| IL-1beta (pg/ml) | 8.3 | 5.2 | <5 |

As can be seen in upper table, the stress hormones DHEA and pregnenolne (see upper text) were decreased. It is recognized that stress is the major cause of Bipolar and Schizophrenia. Also, it is recognized that increase of markers of arterisoslerois is a comorbidity of severe mental diseases. Patients suffering from schizophrenia and Bipolar disease have a 20 to 30 years reduced life expectancy. FSWW08 reduced blood lipids, similarly seen in the study, reported above, with soldiers suffering from PTSD and receiving the same formulation.

Also, any depression and symptoms of Bipolar in the prenatal period was reduced as long as the medication was taken, until one year after delivery.

Any other symptom like sleeping disorder, stress, restless thoughts, aggression, anxiety, as well as suffering frequently from contagious diseases were reduced in the postpartum period for the first time. The patient felt relaxed and it was possible to talk to her without any stress.

The patient complaint frequently about pain in the joints before treatment with FSWW08 and before pregnancy. As can be seen in upper Table, Th1-cytokines TNF-alpha and IL-1b, which are related to inflammation and pain, are reduce din the blood. She did not report any pain or headache any more. She also reported that her female menstrual cycle did return more quickly, compared to last pregnancy. It can be concluded that regular consumption of fermented soy (FSWW08) does reduced Bipolar disease in a pregnant women, during pregnancy and postpartum.

### Example 4

### Ex-Soldier Suffering Form Post Traumatic Stress Disorder (PTSD)

A 43 year Caucasian US citizen, who learned form our new treatment came to our office and reported that he was returning from Iraq 2005 and suffered from Post Traumatic Stress Disorder (PTSD) since 2006. He did stayed in Germany. He was married to a German woman, which he divorced in 2009. He said that he suffered from anxiety attacks, was not interested in having a friends and a female companion. He indicted that he suffered from hostility against man other person that for example he could not stand the women who said at the cashier at his grocery store. Every time he bought something they started to fight with her, because she was dumb, and hostile towards him.

He received a full physical exam in our office, where it became apparent that his immunity was low, because he suffered from herpes labialis, flue symptoms. He indicated that he drank alcohol daily (about 10 bottles), was smoking at least 80 cigarettes a day. He was not consuming other drugs. His blood lipids showed that his triglycerides were seriously elevated, cholesterol and LDL was elevated as well. He was not diabetic though.

He had breathing problems, and started to develop pollen allergy symptoms against certain trees in spring season since he suffered from PTSD.

He had serious sleeping problems and complaint that he was up all night. Blood analysis reveled low testosterone (1.8 ng/ml free testosterone, 25 pg/ml estradiol). Since testosterone is a precursor of estradil and estradiol is low also, it was decided that his sleeping problems were due to low estradiol.

The soldier did complain about constant constipation as well.

He said that he felt depressed lately, but that depression was not his most concern, but anxiety and stress.

The soldier was receiving psycho- and pharmacotherapy by a US army hospital in Germany, but did not feel that this was helpful. He did express respect that US-Army was taken care of them and that they received a lot of respect, but he felt that help was not available.

Treatment with FSWW08

The soldier did received on Feb. 9^{th}, 2010, FSWW08, a bottle a day. Within 10 days he reported that the stress went away, also feelings of anxiety, and that he was not felt a substantial reduction of stressed.

He visited our office on Mar. 23, 2010.

He was diagnosed that his herpes labialis did go away. His breathing was improved, and particularly his sleeping was improved. For the first time since the disease had developed he was experiencing regular sleep at night. We felt that the aggression was diminished and he also reported that he started to do grocery without his regular fights with the lady at the cashier.

His constipation was also lower and he had no stomach pain anymore, what he forgot to report last time.

His breathing problems were also lower, because he could talk without problems.

He visited our office again on Jun. 15^{th} 2010

He reported that for the first time after developing PTSD, he had no allergy symptoms in spring and early summer, like he used to have every year. Also his stomach problems were gone. He still could sleep every night and that this was a major relief. Also his constipation was completely gone.

He was still drinking a lot of beer every day and smoking 80 cigarettes every day.

His aggression was completely gone, and the lady at his grocery store was now his friend. He realized that she was very nice and he planned to spend time in Spain during summer and he was thinking to buy a bottle of good wine for her and buy her present. He said that he was feeling funny about himself, that he did not see that she was pretty. Interestingly he made also compliments about the female doctor in our office, and he explained that he noticed now how pretty she is. When he visited our office for the first time, he did not realize how pretty she was.

Also it was noticed that his appearance changed very much and that he took care of himself now and that his personal hygiene had improved also.

One thing that did not improved was that he had not so many fiends due to his disease and that it was difficult to start a new live.

He visited our office again on Dec. 7^{th} 2010

He informed us that he was going to marry the women at the grocery store, he used to hate when he suffered from PTSD. His appearance had changed. His skin looked very healthy, and he drank half a bottle of (FSWW08) still every day. His inflammatory and viral infections disappeared and he could sleep every night.

### Conclusion,

There cannot be any doubt that the increase of testosterone and estradiol had improved the sleeping habits, because untreated these hormones were too low and increase of both hormones increases sleeping habits. He was not stressed anymore and we noticed that DHEA and pregnenolone were down also, which are related in stress and in all severe mental disease.

He showed decrease of prolactin, a hormone directly correlated with sexuality in man and women. Together with the detected increase of testosterone, it may very well increase sexuality and improve partner relationship. Anxiety disappeared completely in this soldier. He was not depressed, although the outlook on his life was poor. But he was part of an US-Army program.

### Example 5

### The Female Army Veteran, Who Developed Schizophrenia During Pregnancy

We spoke on meetings Vienna about PTSD in army soldiers in Vienna and in were contacted of the Chairman of a Gynecological Hospital in Cologne. He called us for medical and help as a colleague, who reported a case of a pregnant women, who developed signs of schizophrenia during pregnancy.

I visited this hospital and met the woman, who was hospitalized in a mental hospital. The woman was 32 years old. She was pregnant for the first time. Agitation and signs of stress developed before conceiving, because her boyfriend left her. She decided to keep the baby and in the first 3 month, she suffered form stress, depression, vomiting and moodiness. She suffered anxiety disorders and did not recover in the second trimester, what is usually the case in pregnancy.

The gynecologist became aware that the pregnancy was threatened and that a miscarriage was also possible: She suffered form viral infections, particularly flue like symptoms. Since the gynecologist felt that she develops an infection of the uterus, what is frequently seen in miscarriages, he called us in Vienna for help.

The woman was stationed 2007-2008 for almost 2 years in Afghanistan. She did not see death directly, but heart form her fellow comrades. She came back and did stay in the army until present.

She did not show signs of depression or any other mental disturbance. After her boyfriend left her, she was very stressed, because she thought that she could not cope with life, she was anxious and insecure, because her boyfriend brought stability in her life. Clearly, she was falsely diagnosed as depressed, but she was more concerned about having a stable future. She showed signs of stress, and anxiety, when she became pregnant. She did not know who exactly was the father of her child.

When we met her, she was in her 7^{th} month of pregnancy and showed signs of schizophrenia: She could not stand darkness, fled that someone was talking to her, although nobody was in that room. She reported that she heard voices, telling her to leave the hospital. She was very angry sometimes and was difficult to handle her.

Those episodes were provoked when she received visitors, who were difficult to handle and caused stress to her.

The psychiatrist and the gynecologist were concerned giving her medication due to her pregnancy what may danger her baby. She was given a bottle of FSWW06 every day.

Within 7 days her doctors reported dramatic improvements in her well-being: Her stress levels and her anxiety went away. She was not agitated anymore, and could sleep again. She did not hear voices and saw nobody in her room, when there was nobody. Her gynecologist was pleased that infections were stopped, what is a major cause for loosing her baby. Although she was not depressed before, she became cheerful and could talk with nurses and doctors. She was very social again and expressed interested in her baby.

It was decided that after delivery, to give her FSWW08 for another year, because frequently stress returns in form of postnatal depression. We had no compliance problem with the pregnant schizophrenic pregnant women consuming FSWW08, since she felt it was calming her down and did good things for her. She did have a normal postnatal period. She did leave ARMY, due to her mental problems. Since she did receive a lot of supports form her parents and her friends, she could cope with the situation very well.

### Example 6

### The Clinical Trial with Schizophrenic Patients

The neural diathesis-stress model of schizophrenia proposes that stress, through its effects on cortisol production, acts upon a preexisting vulnerability to trigger and/or worsen the symptoms of schizophrenia (Jones et al, Schizophrenia Bulletin, Nov. 14, 2006). Although cortisol level is an established marker for severe mental diseases and has its merits: it is too simplistic to describe the stress response only via cortisol and may be a limited description, because many more hormone are involved and mediating the disease. Stress hormones like DHEA, pregnenolone, and aldosterone are increased, whereas important functional hormones like testosterone, estradiol and particularly androstenediol and androstanediol are decreased. The last two ones, metabolites of androgens, are particularly important, because they regulate immunity, which is severely compromised in most schizophrenic patients.

It is the key feature of this patent that isoflavone formulations in doses similar to soy baby nutrition formula can reduce stress, particularly in schizophrenia, because stress is a major culprit provoking schizophrenic attacks. The key feature of this invention is also that increased stress hormones like DHEA, pregnenolone, can be decreased and those hormones can be increased which are known to calm humans, like estradiol, testosterone etc., These calming hormones which are decreased in schizophrenic patients, can be increased with this invention. It may be stated: Those steroid hormones, which are reduced, are increased from stress hormones. This method kills two birds with one stone.

It was known, that soy isoflavones in doses similar to Asian diets may increase estradiol insignificantly, about 14%, what is insufficient to elicit pharmacologically effects as was explained by Imhofe et al in the Journal Human Reproduction 2009.

The present method, increasing estradiol by taking it from stress hormones, avoids two disadvantages from hormone replacement: in 27% of male patients high doses of oral 2 mg/day fail to increase individual estradiol blood concentration. Second, oral and transdermal doses are relatively high, because patients still suffer from increased stress hormones. Reducing stress hormones and increasing estradiol allows reducing needed estradiol doses, because males had to take 2 mg estradiol, a dose which is above the recommended dose in HRT in women for cancer reasons.

The lower estradiol dose minimizes cancer and feminization risk. Fermented soy also increased substantially testosterone, avoiding the disadvantage of oral estradiol, which decreases testosterone in men and women.

The aim of the present human study was to assess and compare the effectiveness of antipsychotic and a fermented soy product with an established schizophrenic scale, the PANSS in an established treatment facility in Europe. Although virtually all schizophrenic patients suffered from other diseases like, viral infections, inflammatory diseases, and hypertension, it was not possible in this study to document the improvement of accompanying diseases, because we could not interrupt the routine of a major hospital in that are with their normal working routines:

The study was designed to cause no changes in normal routine.

It has to be said, however, that the substantial improvement in metabolic diseases, and immune disturbances was impressive.

We had two important criteria to judge the treatment success, either the effectiveness to prevent readmission to the hospital of fermented soy product is compared with antipsychotics from literature (Johnsen et al. BMC Psychiatry 2010, 10:26), or how quickly would the schizophrenic attacks be reduced (determined by the PANSS scale).

It is the major claim and was shown that severe side effects of antipsychotics can be avoided like increases of cholesterol.

The study was a 24-month, prospective, rather-blind, naturalistic, randomized, head-to head comparison of the effectiveness of three arms a) anti-psychotics, (olanzapine, quetiapine, risperidone, and ziprasidone), b) anti-psychotics with 100 mg soy isoflavone, and 3) a fermented soy product. All patients were recruited from the Division of Psychiatry at Temeshwar, Romania University Hospital with a population of about 400000. The study was approved by the Regional Committee for Medical Research Ethics. Funding of the project was initiated by HAELAN Research foundation, USA.

The Regional Committee for Medical Research Ethics allowed eligible patients to be included before informed consent was provided, thus entailing a clinically relevant representation in the study. The disqualification of the most gravely ill patients from participating in trials represents an ethical dilemma; however, as these patients will most likely receive the drugs once they are approved for marketing, despite the lack of evidence from this population. Trial inclusion of patients without informed consent is justifiable on 2 conditions: That no other context exists in which the research

questions can be answered, and that all patients get clear clinical benefit from whatever treatment they are allocated to.

These criteria are fulfilled in some mental conditions from which important studies have been published. Patients (age≧18 years) were eligible for the study if they were admitted to the emergency ward for symptoms of psychosis as determined by a score of ≧4 on one or more of the items Delusions or Hallucinatory behavior, Grandiosity, Suspiciousness/persecution, or Unusual thought content in the Positive and Negative Syndrome Scale (PANSS), and were candidates for the three arms of the outlined study, either oral antipsychotic drug therapy, antipsychotic drug therapy with a 100 mg soy isoflavone, or a fermented soy product. Eligible patients met ICD-10 diagnostic criteria for schizophrenia, schizoaffective disorder, schizophreniform disorder, brief psychotic episode, delusional disorder, drug-induced psychosis, and major depressive disorder with psychotic features. The diagnoses were determined by experienced clinicians. Patients were excluded from the study if they were unable to use oral antipsychotics, were suffering from manic psychosis, were unable to cooperate reliably during investigations, did not understand spoken Romanian language, were candidates. Patients with drug-induced psychoses were included only when the condition did not resolve within a few days and when antipsychotic drug therapy was indicated. The evidence thus far shows that to prospectively predict which antipsychotic might be optimal for a given patient with regards to effect and tolerability is not possible, and that antipsychotic therapy currently involves a trial and error approach [15]. A prior history of antipsychotic drug use may provide some information, though. Taking these factors into account the protocol mimicked the normal clinical situation in which oral antipsychotic drug therapy is initiated, with one exception: At admission, a sealed and numbered envelope was opened by the attending psychiatrist and then the patient was offered one amount lined as a) b) or c) above, in a random sequence of the first-line antipsychotics in Romania-olanzapine,

quetiapine, risperidone, or ziprasidone.

The randomization was open to the treating psychiatrist or physician and to the patient. Both the treating clinician and/or the patient could discard the SGA listed as number 1 on the list because of medical contraindications for the use of, or prior negative experiences with the drug, however, and the next on the list could be chosen.

### Assessments

Study visits were at baseline, and then followed every month, up to two years. One trained investigator performed all assessments. Before inclusion, the investigator interviewed eligible patients, using the PANSS, Scale.

The patients received a physical examination by the admitting physician, and standard blood samples were collected according to the hospital's routine. At discharge from the hospital or at 6 weeks if not discharged, the tests and examinations were repeated by the rater who was unaware of the treatment and serum level measurements of the antipsychotics and blood analytic was conducted.

Thus far, all investigations and tests were part of the hospital's routine for the management of patients suffering from psychosis and became part of the patient's medical record. At this point, the patients were asked for informed consent to be contacted and included in the follow-up project.

At follow-up visits up to two years measures of psychopathology, function, and tolerability, as well as clinical and laboratory assessments were repeated by the rater blind to treatment. Symptoms were assessed by the PANSS.

### Results

A total of 168 patients were allocated to randomized sequences of the first-line treatment. The antipsychotics listed as 1 defined the randomization groups (Rags). A total of 68 patients received the antipsychotics (42 men, 26 women), whereas 52 were assigned to the arm, combining antipsychotics with 100 mg soy isoflavones (39 men, 13 women), and 78 were assigned to the arm receiving fermented soy (52 men, 26 men).

Baseline demographics and clinical characteristics will be presented elsewhere, but are not different from each other in each group and from literature (Johnson et al. BMC Psychiatry 2010, 10:26).

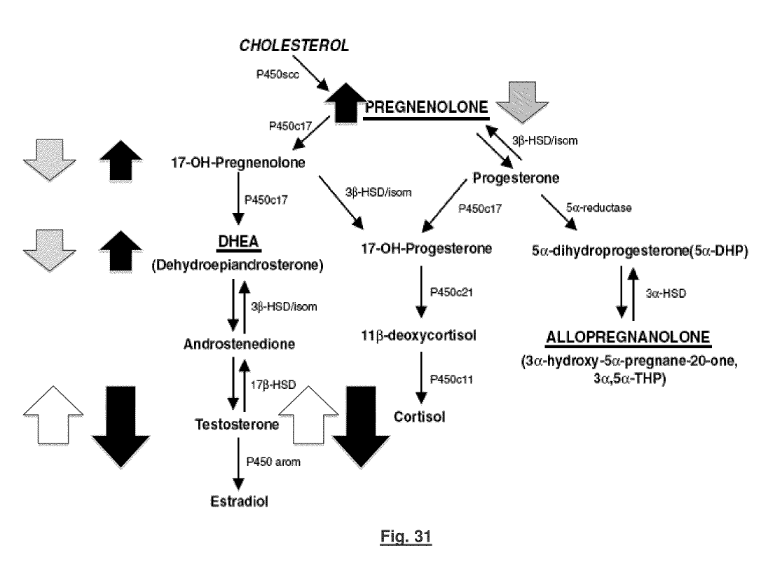

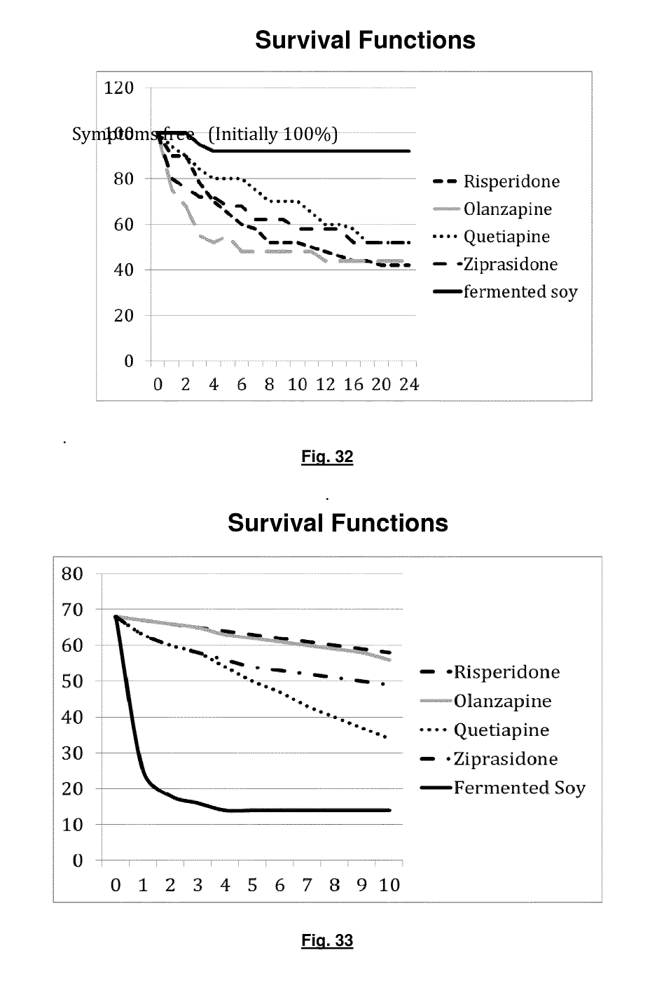

Outcomes related to symptom reduction and increased functioning are shown in FIGS. 32 and 33. There were small differences among antipsychotics (taken from literature (Johnson et al 2009) in reducing the PANSS total score and the positive sub score.

As can be seen fermented soy had a significant effect to reduce re-admittance to the hospital and to calm down patients (FIG. 32).

FIG. 32: Time to rehospitalization=Time (month) until rehospitalization after discharge from index admission. Men and women were not separated. Data from different antipsychotics were taken from literature (Johnsen et al. BMC Psychiatry 2010, 10:26).

As can be seen in FIG. 32, anti-psychotics and fermented soy showed considerable differences in preventing readmission to return to the hospital for treatment of schizophrenic episodes. Although there are differences in the type of antipsychotics, they seam to exhibit similar features.

The symptom reduction of schizophrenic attacks by fermented soy was unexpected and a different mechanism seems to be involved (FIG. 32). Two patients had to reappear in the arm with fermented soy to the hospital, because they had difficulties to follow the instructions. No side effect of fermented soy product was reported, because it is a nutritional product in Asia.

Differences between antipsychotics have been identified in the literature and are depicted in FIG. 32. Although they are statistically significant, they are small compared to effects of fermented soy to reduce schizophrenic episodes based (FIG. 32,33).

FIG. 33 depicts the onset of pharmacologically relevant effect reducing schizophrenia attacks. As can be seen in FIG. 33, fermented soy did reduce schizophrenic symptoms relatively quick and long lasting, whereas the effect of antipsychotics take a long period of several month to produce substantial effects. Data from the fermented soy arm in FIG. 33were experimentally determined in this study.

FIG. 33: Reduction of PANSS total score. (Data from antipsychotics were taken from the literature: Johnsen et al. BMC Psychiatry 2010, 10:26. Data from the fermented soy arm were experimentally determined.

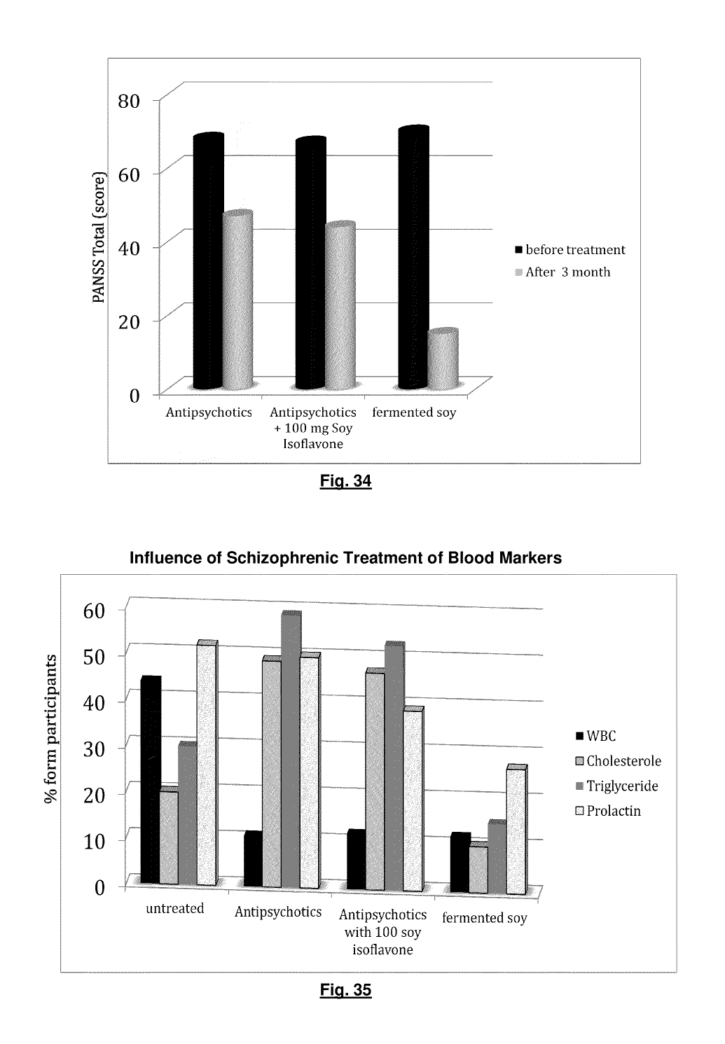

To investigate whether soy isoflavone concentration influences the efficacy of reducing schizophrenic attacks, daily consumption of 100 mg soy isoflavones to the normal antipsychotic intake was added. As can be seen daily consumption of 100 mg soy isoflavone did change the outcome, measured by the PANSS (FIG. 34): Clearly the addition of 100 mg soy isoflavones capsules did not change the efficacy of antipsychotics and was not statistically different from average score of each other (FIG. 34) after 3 month, based on PANSS. There is no time dependent different curve between antipsychotics and antipsychotics and soy isoflavones (will be depicted elsewhere). Since it was never reported in the literature that the addition of soy has ever reduced the efficacy of antipsychotics, and its use is global, we conclude that there was no interaction between any antipsychotic and soy isoflavones.

Since antipsychotics are known to change unfavorably cholesterol and triglycerides, the three arms, antipsychotics, antipsychotics plus 100 mg soy isoflavones, and fermented soy were compared (FIG. 35). As can also be seen in FIG. 4, antipsychotics have negative effects on cholesterol and triglycerides, whereas they have favorable effect on white blood counts confirming results from the literature. Soy isoflavones in daily doses of 100 mg do not improve the unfavorable effect of antipsychotics statistically significantly (FIG. 35). FIG. 4 depicts fractions in % from total participants, whose value are outside the recommended normal range. As was expected, WBC is reduced by antipsychotics as well as by fermented soy.

Although there are differences in reducing or increasing prolactin depending on the chosen antipsychotic, fermented soy did reduce increased prolactin in schizophrenic patients about half (FIG. 5). This was shown for the first time ever.

FIG. 34: Comparison of reduction of average schizophrenic symptoms based on PANSS after 3 month in 3 different arms of treatment.

FIG. 35: Effect of schizophrenic treatment on selected blood markers, discussed in schizophrenic patients. Average values were determined after 3 month of treatment. Depicted are fractions in % from total whose value is outside the recommended normal range.

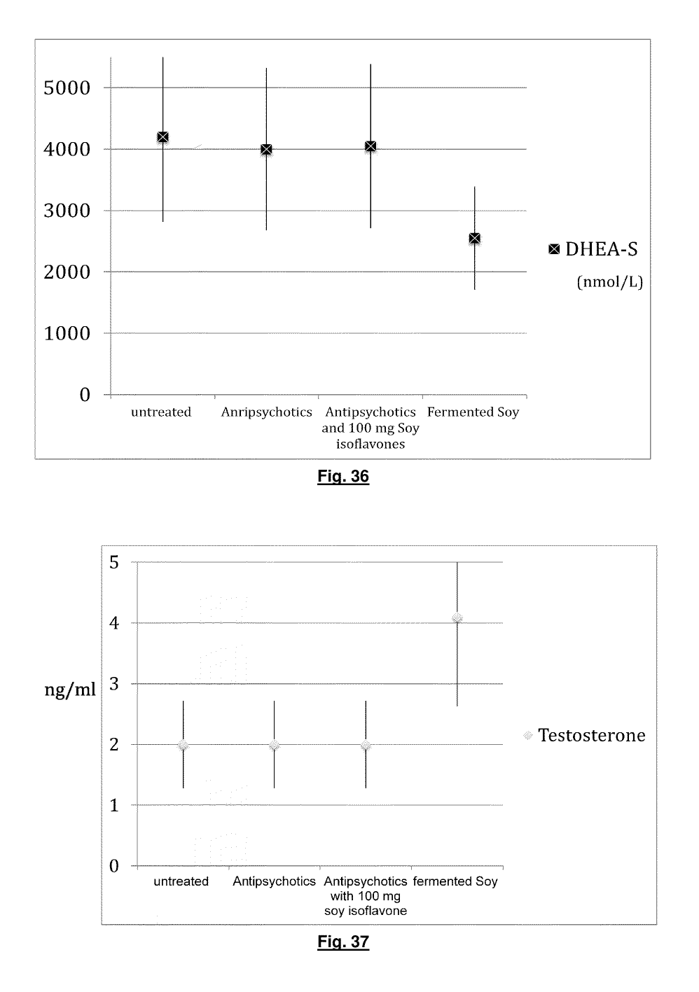

As was demonstrated above, in the study with soldiers suffering from PTSD, fermented soy was able to reduce hormones like DHEA-S (FIG. 36), progesterone, aldosterone, pregnenolone, although not depicted here, but will be reported elsewhere. Pregnenolone, and cholesterol (see also FIG. 11) has to be considered as stress hormone and are important markers in schizophrenia. As can be seen in FIG. 36, antipsychotics had no significant influence to reduce stress hormones; also adding 100 mg soy isoflavones was insignificant to improve the situation. Fermented soy however did reduce elevated stress hormone levels like DHEA-S by about half (FIG. 36).

On the contrary, hormones like testosterone (FIG. 37), and estradiol (FIG. 38) were increased simultaneously by fermented soy.

Adjunctive use of estrogen therapy has been shown to be effective in enhancing the treatment of schizophrenia in women. In men, consideration of estrogen therapy has been impacted by concerns of feminizing side effects, however, clinical trials of the use of estrogen in treating prostate cancer, bone density loss and even aggression and psychosis in dementia or trauma. A recent trial has shown that oral estradiol in doses of 2 mg estradiol valeriate in schizophrenic men raises estradiol levels, shows however two drawbacks: First, despite this high dose, 27% failed to increase estradiol levels, which correlated with failed efficacy, and most importantly did reduce testosterone, which is lower in schizophrenic patients already. This lowers in most cases the sex drive. Nevertheless, Results demonstrated for estradiol participants a more rapid reduction in general psychopathology. Therefore the role of adjunct estradiol therapy has to be expanded with a better alternative than giving high doses of external estradiol. It is better to take it from elevated stress hormones, which reduce them, and balance other hormones that reduce stress as well. The required doses for treatment are thereby lowered and reduce feminization and cancer risks.

FIG. 36: The effect of treatment with various regimens on stress hormone DHEA-S plasma levels after 3-month treatment in schizophrenic patients in the acute phase. For more explanation see text. Normal DHEA-S range between 2500 and 4500 nmol/L.

FIG. 37: The effect of treatment with various regimens on testosterone plasma levels after 3-month treatment in schizophrenic patients in the acute phase. For more explanation see text. Normal range between 2500 and 4500 nmol/L. Normal range for testosterone is between 2 and 7 ng/ml.

FIG. 38: The effect of treatment with various regimens on estradiol plasma levels after 3-month treatment in schizophrenic men in the acute phase. For more explanation see text. Normal range of Estradiol in men is between (10-82 pg/ml).

As was recently shown that a reduction of estradiol below 37 pg/ml in men does increase the risk of developing osteoporosis by 87% (Clapauch, Arq Bras Endocrinol Metabol. 2009 53:1020-5.) and it has been concern of psychiatrist that schizophrenic patients suffer from osteoporosis. It has been established that total estradiol, rather than testosterone levels, predicts osteoporosis in aging men.

Recently it was found that antidepressants, benzodiazepins as well as antipsychotics increase osteoporosis risk. We quote from: Bolton J Clin Psychopharmacol. 2011 31:56-60.

Selective serotonin reuptake inhibitors (adjusted odds ratios, 1.46; 95% confidence interval [Cl], 1.25-1.69), atypical antipsychotics (AOR, 1.55; 95% Cl, 1.06-2.28), and benzodiazepines (AOR, 1.17; 95% Cl, 1.06-1.29) were associated with higher risk of osteoporosis. Tricyclic antidepressants were associated with lower odds of osteoporosis (AOR, 0.57; 95% Cl, 0.49-0.65). These drug effects were independent of mental illness diagnoses including depression (AOR, 0.86; 95% Cl, 0.75-0.98) and schizophrenia (AOR, 1.98; 95% Cl, 1.04-3.77).

Although one may ask, how did fermented soy interact with cortisol?, the classical marker for HPA disturbances, we have to say the following:

From theory, one would expect a decrease of cortisol. An increase of cortisol in schizophrenic patients would reveal that depression is an additional disease, overpowering the decrease.

Young schizophrenic patient show frequently decreased cortisol levels (Hempel, et al. Psychiatry Clin Neurosci. 2010 October; 64(5):548-54), whereas many reports show no influence on cortisol levels at all, whereas old schizophrenic patients in a unfavorable social environment show an increase of cortisol level. Studies showing no change in cortisol may be interpreted as a mixture between schizophrenic patients and schizophrenic patients with accompanying depression have been investigated.

Fermented soy did reduce cortisol levels, indicating that depression as well as schizophrenia was improved. Since we did not discriminate between schizophrenia and depression, we skip discussion of cortisol.

Discussion of normalization of hormones over a whole range improves general health as was indicated by the risk of osteoporosis. However improvements in general health was imminent in this trial.

### Example 7

Investigation on Gene expression changes in patients suffering from severe mental diseases are getting more attention to monitor the therapeutic success as well as developing markers for mental diseases, which have not been established yet. Investigations in monocytes in PTSD patients receiving fermented soy for 3 month have been conducted. The mechanism of this therapeutic effect is not clear. The current study examined molecular changes induced in human peripheral mononuclear cells (PMC) in order to get insight into its mechanism of action. Gene expression profile of PMC from PTSD-treated patients was examined before additional daily fermented soy consumption for 3 month after. Gene expression patterns screened with a cDNA array, comprising 22000 genes (Affymetrix Genome-Wide Human SNP Array 6.0, Affymettrix, Santa Clara, Calif., USA). Genes related to PTSD were assayed and the results verified by real-time RT-PCR.

As can be seen, PTSD patients suffer from lower Estrogen receptor alpha as well as beta gene expression. Also, the androgen receptor is less expressed in PTSD patients, comparedto normal men. If PTSD patients consume fermented soy, a considerable increase of Estrogen receptor beta gene expression, as well as the androgen receptor is increased. Surprisingly the glucocorticoid receptor is decreased.

Gene expression studies do corroborate the plasma hormones profiles, since testosterone, estradiol, and particularly cortisone is decreased.

### Example 8

The here tested fermented soy formulation (SOY-IQ, also referred to herein as FSWW08) was patented (U.S. Pat. No. 4,877,739). This invention makes use of autogenic antiammonia azotobacter, especially a mutant strain, azotobacter, 851 yellow induced from Azotobacter vinelandii, 851 yellow, as inoculum in the industrial fermentation, can produce single cell protein riched in Se, Zn, vitamins and bacterial manure by utilization of the atmospheric nitrogen.

At present, scientists all over the world are studying the bio-azotofication. They hope that autogenic azotobacter will directly transform the atmospheric nitrogen into protein and fertilizer to ease the contradiction between the population and the food in the world today. Because the azotobacter in nature is not antiammonia azotobacter, it is of little value of spreading and applying.

The invention offers a group of azotobacters, which have capability of anitammonia nitrogen-fixation, and have higher azotase activity. They can strongly fix the atmospheric nitrogen in the presence of the nitrogen-containing compound in the medium or the environment. In order to produce products, which are valuable to industrial production such as single proteins containing Se, Zn, lots of vitamin C.D.K., vitamin E, bacterial manure and a series of products, the medium, which is suitable for growth of the strain has been devised in this invention.

One of the strains according to this invention is the antiammonia azotobacter 851 yellow. It has remarkably the ability of antiammonia nitrogen-fixation. The strains were bred through breeding by artificial mutagenesis from Azotobacter vinlandii because of glutamine synthetase gene mutation. The difference between the mutant strain and its parents is that the former can keep higher azotase activity in the medium containing nitrogen, even in the presence of (NH₄)₂ SO₄.

The strain can make use of starch directly. All substances that contain starch such as potato, sweet potato, corn etc. can be used as the carbon source for 851 yellow to grow. Using “851” yellow as productive inoculum, protein rich in Se and Zn single cell protein containing various vitamins can be produced in the starch culture medium according to this invention. In 100 ml of the culture fluid, the amount of the total amino acid is 0.2-2 g; organic selenium, 0.5-5 ppm; Zn 5-20 ppm. In 100 ml of the culture fluid of this strain, the vitamin E is in an amount of 8.2-114 mg (results of laboratory), when it is incubated n the mannitol yeast extract medium, and mannitol is used as carbon source. This strain is also suitable for producing the single cell protein-containing vitamin by using the waste molasses as the carbon source. The amount of total amino acids in the culture fluid is 200-500 mg/100 ml.

The ant ammonia azotobacter according to this invention has overcome the disadvantage that azotobacter can not strongly fix the atmospheric nitrogen in the presence of the compound nitrogen. Thus, this strain is not only the better strain for producing bacterial manure, but also the industrial strain for producing the single cell proteins rich in Se-, Zn- and containing various vitamins.

The media devised and employed are: The starch medium dry starch 1-2% fresh substances containing starch (sweet potato, potato etc.) 10-20% or dry substances containing starch (corn etc.) 1-10% yeast extract 0.04-0.08% K₂ HPO₄ 0.05%0.1% MgSO₄ 0.02-0.04% NaCl 0.02-0.04% CaCO₃ 0.25-0.5% sodium molybdate 10-20 ppm boric acid 10-20 ppm Na₂ SeO₃ 5-20 ppm ZnSO₄ 5-20 ppm 2. The mannitol medium mannitol 1% KH₂ PO₄ 0.02% MgSO₄ 0.02% NaCl 0.02% CaSO₄ 0.02% CaCO₃ 0.25-0.5% 3. The mannitol-yeast extract medium mannitol 1% yeast extract 0.04% K₂ HPO₄ 0.05% MgSO₄ 0.02% NaCl 0.02% CaCO₃ 0.25-0.5% sodium molybdate 5-10 ppm boric acid 5-10 ppm 4. The waste molasses medium molasses 2-3% CaCO₃ 0.25%-0.5% sodium molybdate 5-10 ppm FeSO₄ 5-10 ppm.

The antiammonia azotobacter, 851 yellow as the productive strain is incubated for 86-110 hrs. in the fermentation tank which contains the medium of starch (corn powder) (semi-continuous fermentation). Ventilating Amount: 1:0.6-1.2, Stirring speed 100-300 rpm, Culture Temperature 25°-30° C., At the end of incubation the total amount of amino acid is 0.5-1 g; organic Se, 5 ppm; Zn, 20 ppm and vitamin C, E, B₁, B₂, B₁₂, A, K, D and nicotinic acid etc. in 100 ml of the culture fluid. After the culture fluid is dried directly at 80° C. the amount of dry substances is 5-12%, in which the protein is up to 15-30%.

### Example 9

This invention refers to U.S. Pat. No. 6,214,875: Anticancer effects of specific branched-chain fatty acids and related production process (incorporated herein by reference—formulas not shown here can be found in that patent).

The present invention relates to a group of compounds, i.e., specific branched-chain saturated and unsaturated fatty acids, with significant anticancer activities. The branched-chain saturated fatty acids can be described by the formula:##STR1## where n and m are independently integers, and n+m is between 0 and 46, inclusive. Preferably, m is 0 or 1, and n is 7-16.

The branched-chain unsaturated fatty acids of the present invention have the above formula, except that m or n is at least 2, and at least one CH.sub.2-CH.sub.2 group in (CH.sub.2).sub.m or (CH.sub.2).sub.n is replaced with a CH.dbd.CH group. The term “I so-Cx”, as used in the present invention, means a branched-chain saturated fatty acid having x carbons and n=x−4, m=0 in the above formula. The term “anteiso-Cx” means the compound having x carbons and n=x−5, m=1 in the above formula. For example, 13-methyltetradecanoic acid is expressed as “iso-C15” and has the formula ##STR2##

12-methyltetradecanoic acid is expressed as “anteiso-C15” and has the formula:
##STR3##. An example of an unsaturated branched-chain fatty acid of the present invention is 15-methylhexadecenoic acid: ##STR4## otherwise known as iso-17:1 .omega.9c.

The present invention also includes pharmaceutically acceptable salts of said saturated and unsaturated branched-chain fatty acids, which are obtained by reaction with inorganic bases, such as sodium hydroxide, and have the ability to inhibit fatty acid synthesis.

The present invention also includes pharmaceutically acceptable lipoproteins of said saturated and unsaturated branched-chain fatty acids, which are obtained by conjugation with proteins, including polypeptides and oligopeptides, and have the ability to inhibit cancer cell growth. Such lipoproteins are well known in the art. The specific branched-chain fatty acids of the present invention can be obtained by, but not limited to, isolation from fermentation or incubation products using specific bacteria, or by electrochemical synthesis, or by extraction from natural materials. Having generally described this invention, a further understanding can be obtained by reference to certain specific examples, which are provided herein for purposes of illustration only and are not intended to be limiting unless otherwisespecified.
10-methylundecanoic acid (iso-C12),
11-methyllauric acid (iso-C13),
12-methyltridecanoic acid (iso-C14),
11-methyltridecanoic acid (anteiso-C14),
12-methyltetradecanoic acid (anteiso-C15),
14-methylpentadecanoic acid (iso-C16),
13-methylpentadecanoic acid (anteiso-C16),
15-methylpalmitic acid (iso-C17),
16-methylheptadecanoic acid (iso-C18),
15-methylheptadecanoic acid (anteiso-C18),
17-methylstearic acid (iso-C19),
18-methylnonadecanoic acid (iso-C20).

### Example 10

The relationship between streptococcus infection and severe mental depression [79] has long been recognized. Streptococcus infections are also responsible for what is called “flesh eating bacteria”, were bacteria cause permanent wound healing disturbances.

It was found that a patient, (male 32 years old, resident of New Orleans, USA) with severe mental depression, PTSD and a severe wound-healing problem was treated with fermented soy (FIG. 42). Within 3 month a complete healing as well as an instantaneous improvement of wellbeing was recorded by treating physicians. The patient did receive the streptococcus infection (streptococcus pylorii) in the after mass of Hurricane Katrina. Severe wound healing was detected and a complete amputation of the leg was avoided after the patient did show improvement of the disease.

### Example 11

Parturition is the process by which the fetus is expelled from the uterus to the extra uterine environment. Parturition results from a complex interplay of maternal and fetal factors. It is not known that besides many factors, the HPA axis, which is governing the mental wellbeing, is also a key player in controlling term and preterm birth. Stress, which is a major factor in causing preterm birth, is mediated by disturbances of the HPA axis (FIG. 38).

It is a key finding of our patent that the same biological mechanisms governing severe mental disturbances also determine severe complications of pregnancy like preeclampsia, infections, hypertension and preterm birth. Also postnatal depression is more likely a postnatal stress disorder than a depressive disorder and resembles PTSD.

Preterm birth, where there is asynchrony between the labor process and fetal maturation, occurs in 8-10% of all pregnancies, and its incidence has changed little in the past 40 yr (1). Indeed, factors such as low socioeconomic status of some inner-city populations, the tendency for women to choose to start a family at an older age, and the impact of fertility treatment are contributing to an increase in the incidence of preterm delivery (2, 3). Improved neonatal care, however, continues to reduce the mortality rate due to pre maturity, although preterm birth remains the primary cause of neonatal death. In North America, the cost of caring for infants in the neonatal intensive care nursery during the first months of life has been estimated at $5-6 billion annually (3).

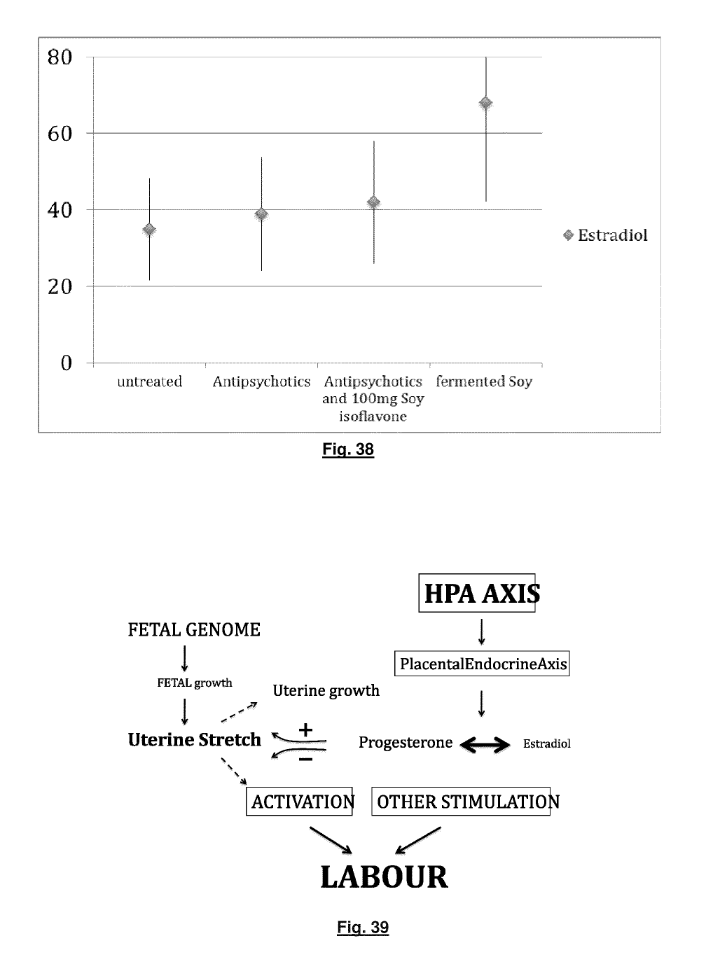

FIG. 39: The onset of labor is dictated by the fetal genome proceeding through either a fetal growth pathway with increases in uterine stretch or fetal endocrine pathway involving activation of the fetal HPA axis. These two arms are not independent because changes in progesterone and estrogen modulate the ability of uterine stretch to increase expressions of genes associated with myometrial activation.

It can be seen that the hypothalamus pituitary adrenal (HPA) axis is directly involved in premature labor, pregnancy depression and postnatal depression. (FIG. 43). Every application of fermented soy will lead to improvement of pregnancy depression, risk of miscarriage or postnatal depression.

### Example 12

The last three pages of this disclosure documents some of the analytical results from the batch, which was investigated in the clinical trial.

### REFERENCES

1. Sperner-Unterweger B. Immunological aetiology of major psychiatric disorders: evidence and therapeutic implications. Drugs. 2005; 65(11):1493-520.
2. Müller N, Riedel M, Gruber R, Ackenheil M, Schwarz M J. The immune system and schizophrenia. An integrative view. Ann N Y Acad. Sci. 2000; 917:456-67.
3. Strous R D, Shoenfeld Y. Schizophrenia, autoimmunity and immune system dysregulation: a comprehensive model updated and revisited. J Autoimmun. 2006 September; 27(2):71-80.
4. Gaughran F. Immunity and schizophrenia: autoimmunity, cytokines, and immune responses. Int Rev Neurobiol. 2002; 52:275-302.
5. Rogers D P, Goldsmith C A. Treatment of schizophrenia in the 21st Century: beyond the neurotransmitter hypothesis. Expert Rev Neurother. 2009 January; 9(1):47-5
6. Muench J, Hamer A M. Adverse effects of antipsychotic medications. Am Fam Physician. 2010 Mar. 1; 81(5):617-22.
7. Domenici E, D R Wille, F Tozzi, I Prokopenko, S Miller, A McKeown, C Brittain, D Rujescu6, I Giegling, C W Turck, F Holsboer, E T Bullmore, L Middleton, E Merlo-Pich, R C Alexander, P Muglia. Plasma Protein Biomarkers for Depression and Schizophrenia by Multi Analyte Profiling of Case-Control Collections. PLoS One. 2010 Feb. 11; 5(2):e9166.
8. NATIONAL INSTITUTE OF MENTAL HEALTH.http://www.nimh.nih.gov/health/publications/the-numbers-count-mental-disorders-in-america/index.shtml
9. Kessler R C, Chiu W T, Demler O, Walters E E. Prevalence, severity, and comorbidity of twelve-month DSM-IV disorders in the National Comorbidity Survey Replication (NCS-R). Archives of General Psychiatry, 2005 June; 62(6):617-27.
10. Nanri A, Kimura Y, Matsushita Y, Ohta M, Sato M, Mishima N, Sasaki S, Mizoue T.
Dietary patterns and depressive symptoms among Japanese men and women. Eur J Clin Nutr. 2010 May 19. [Epub ahead of print]
11. Lash T L, Cronin-Fenton D, Ahern T P, Rosenberg C L, Lunetta K L, Silliman R A, Hamilton-Dutoit S, Garne J P, Ewertz M, Sørensen HT, Pedersen L. Breast cancer recurrence risk related to concurrent use of SSRI antidepressants and tamoxifen. Acta Oncol. 2010 April; 49(3):305-12.
12. Colton C W, Manderscheid R W. Congruencies in increased mortality rates, years of potential life lost, and causes of death among public mental health clients in eight states. Prey Chronic Dis [serial online] 2006 April [date cited]. Available from: URL: http://www.cdc.gov/pcd/issues/2006/apr/05_{—}0180.htm.
13. Tucker P, Jeon-Slaughter H, Pfefferbaum B, Khan Q, Davis N J. Emotional and biological stress measures in Katrina survivors relocated to Oklahoma. Am J Disaster Med. 2010 March-April; 5(2):113-25.
14. Kozumplik O, Uzun S, Jakovljević M. Metabolic syndrome in patients with psychotic disorders: diagnostic issues, comorbidity and side effects of antipsychotics. Psychiatr Danub. 2010 March; 22(1):69-74.
15. Reiger et al. Archives of General Psychiatry 1993; 50:85-94.
16. Durso, Employee Benefit News, December 2004.
17. Miller C A, Golden N H. An introduction to eating disorders: clinical presentation, epidemiology, and prognosis. Nutr Clin Pract. 2010 April; 25(2):110-5.
18. Strous R D, Maayan R, Kaminsky M, Blumensohn R, Weizman A, Spivak B. DHEA and DHEA-S levels in hospitalized adolescents with first-episode schizophrenia and conduct disorder: a comparison study. Eur Neuropsychopharmacol. 2009 July; 19(7):499-503.
19. Strous R D, Maayan R, Lapidus R, Goredetsky L, Zeldich E, Kotler M, Weizman A. Increased circulatory dehydroepiandrosterone and dehydroepiandrosterone-sulphate in first-episode schizophrenia: relationship to gender, aggression and symptomatology. Schizophr Res. 2004 Dec. 1; 71(2-3):427-34.
20. Loria R M. Beta-androstenes and resistance to viral and bacterial infections. Neuroimmunomodulation. 2009; 16(2):88-95
21. Barbaccia M L, Lello S, Sidiropoulou T, Cocco T, Sorge R P, Cocchiarale A, Piermarini V, Sabato A F, Trabucchi M, Romanini C. Plasma 5alpha-androstane-3alpha,17betadiol, an endogenous steroid that positively modulates GABA(A) receptor function, and anxiety: a study in menopausal women. Psychoneuroendocrinology. 2000 October; 25(7):659-75.
22 Zhang T, Pan W, Takebe M, Schofield B, Sampson H, Li X M. Therapeutic effects of a fermented soy product on peanut hypersensitivity is associated with modulation of T-helper type 1 and T-helper type 2 responses. Clin Exp Allergy. 2008 November; 38(11):1808-18.22
23. Rybalko S L, Anisimova IuN, Maksimenok E V. Anti-HIV effect of soy products and possibility of their usage as medicinal and prophylactic nutrition. Lik Sprava. 2008 October-December; (7-8):63-9.
24. Laugero et al. Endocrinology 2001; 142:2796-2804.
25. Laugero et al. Endocrinology 2002; 143:4552-4562.
26. Dallman et al. Proc. Natl. Acad. Sci. USA 2003; 100:11696-11701.
27. Wong et al. Proc. Natl. Acad. Sci. USA 2000; 97:325-330.
28. Sarnyai et al. Pharmacol. Rev. 2001 53:209-243.
29. Heinrichs et al. Baillieres Best Pract. Res. Clin. Endocrinol. Metab. 1999 13:541-554.
30 Chrousos, Int. J. Obes. Relat. Metab. Disord. 2001 24:S50-S55.
31. American Diabetes Association et al. Diabetes Care 200427; 596-601.
32. Panagiotopoulos C, Ronsley R, Elbe D, Davidson J, Smith D H. First do no Harm: Promoting an Evidence-Based Approach to Atypical Antipsychotic use in Children and Adolescents. J Can Acad Child Adolesc Psychiatry. 2010 May; 19(2):124-37.
33. Krahn et al Brain Research Bulletin 198617:285-289;
34. Smagin et al. Am. J. Physiol. 276:R1461-1468; Koob (1999) Biol. Psychiatry 46:1167-1180.
35. Dallman et al. Proc. Natl. Acad. Sci. 2003 USA 100:11696-11701).
36. Chrousos et al. (2000) Int. J. Obes. Relat. Metab. Disord. 24:S50-S55;
37. Gillespie C F, Bradley B, Mercer K, Smith A K, Conneely K, Gapen M, Weiss T, Schwartz A C, Cubells J F, Ressler K J. Trauma exposure and stress-related disorders in inner city primary care patients. Gen Hosp Psychiatry. 2009 November-December; 31(6):505-14.
38. http://edition.cnn.com/2002/US/07/26/army.wives/index.html
39. http://www.youtube.com/watch?v=sFC8mNdxV0
40. Krysinska K, Lester D. Post-traumatic stress disorder and suicide risk: a systematic review. Arch Suicide Res. 2010; 14(1):1-23.
41. “National trends in Psychotropic Medication Polypharmacy in Office-Based Psychiatry.” Archives of General Psychiatry. January 2010. 2Nemeroff, C B., DeVane, C L., Pollock, B G. “Newer antidepressants and the cytochrome P450 system.” Am J Psychiatry 1996; 153:311-320 3. Disalver, S., Greden, J., Snider, R. “Antidepressant Withdrawal Syndromes: Phenomenology and Pathology.” January 1987:2 (1). 4. Smoller J., Allison, M. et al. “Antidepressant Use and Risk of Incident Cardiovascular Morbidity and Mortality Among Postmenopausal Women in the Women's Health Initiative Study.” Arch Intern Med, 2009; 169 (22): 2128-2139. 5. FDA Public Health Advisory: REPORTS OF SUICIDALITY IN PEDIATRIC PATIENTS BEING TREATED WITH ANTIDEPRESSANT MEDICATIONS FOR MAJOR DEPRESSIVE DISORDER (MDD). Oct. 27, 2003. 6. Pharmacologic management of psychiatric illness during pregnancy: dilemmas and guidelines. Am J Psychiatry 1996; 153:592-606. 7. Caley C F. “Extrapyramidal reactions and the selective serotonin-reuptake inhibitors.” Ann Pharmacother. 1997 December; 31(12):1481-9. 8. Eleanor Kibrick and Reginald G. Smart, “Psychotropic Drug Use and Driving Risk.” Can Fam Physician. 1972 Oct.; 18(10): 47-52.
42. Hopkins S J. Central nervous system recognition of peripheral inflammation: a neural, hormonal collaboration. ACTA BIOMED 2007; 78; Suppl 1: 231-247
43. Ayada K, K Yokota, Y Kawahara, Y Yamamoto, K Hirai, T Inaba, M Kita, H Okada, K Yamamoto, K Oguma. Immune Reactions Against Elongation Factor 2 Kinase: Specific Pathogenesis of Gastric Ulcer from Helicobacter pylori Infection Clinical and Developmental Immunology, Volume 2009, Article ID 850623, 10 pages doi:10.1155/2009/850623. 2) Iwata A, Watanabe N, Oya Y, Owada T, Ikeda K, Suto A, Kagami S, Hirose K, Kanari H, Kawashima S, Nakayama T, Taniguchi M, Iwamoto I, Nakajima H. Protective roles of B and T lymphocyte attenuator in NKT cell-mediated experimental hepatitis. J Immunol. 2010 Jan. 1;184(1):127-33. 3) Takaki A, Tatsukawa M, Iwasaki Y, Koike K, Noguchi Y, Shiraha H, Sakaguchi K, Nakayama E, Yamamoto K. Hepatitis C virus NS4 protein impairs the Th1 polarization of immature dendritic cells. J Viral Hepat. 2009 Oct. 4. [Epub ahead of print]. 4) Iijima N, M M Linehan1, M Zamora, D Butkus, R Dunn, M R Kehry, T M Laufer, and A Iwasaki. Dendritic cells and B cells maximize mucosal Th1 memory response to herpes simplex virus. The Journal of Experimental Medicine, Vol. 205, No. 13, 3041-3052
44. Pace T W, Miller A H. Cytokines and glucocorticoid receptor signaling. Relevance to major depression. Ann N Y Acad. Sci. 2009 October; 1179:86-105.
45. Newcombe J W, Metabolic Syndrome and Mental Illness. Am J Manag Care. 2007;13:S170-S177
46 Colton C G, et al, Congruencies in Increased Mortality Rates, Years of Potential Life Lost, and Causes of Death Among Public Mental Health Clients in Eight States. Prey Chronic Dis. 2006 April; 3(2):A42.
47. Domenici E, et al, Plasma Protein Biomarkers for Depression and Schizophrenia by Multi Analyte Profiling of Case-Control Collections. PLoS ONE 5(2): e9166. doi:10.1371/journal.pone.0009166
48. Cohen M H, Fabri M, Cai X, Shi Q, Hoover D R, Binagwaho A, Culhane M A, Mukanyonga H, Karegeya D K, Anastos K. Prevalence and predictors of posttraumatic stress disorder and depression in HIV-infected and at-risk Rwandan women. J Womens Health (Larchmt). 2009 November; 18(11):1783-91.
49. Elklit A, Petersen T. Exposure to traumatic events among adolescents in four nations. Torture. 2008; 18(1):2-11.
50. McAllister T W. Psychopharmacological issues in the treatment of TBI and PTSD. Clin Neuropsychol. 2009 November; 23(8):1338-67
51. Chan D, Cheadle A D, Reiber G, Unutzer J, Chaney E F. Health care utilization and its costs for depressed veterans with and without comorbid PTSD symptoms. Psychiatr Serv. 2009 December; 60(12):1612-7
52. Costa-Requena G, Gil F. Posttraumatic stress disorder symptoms in cancer: psychometric analysis of the Spanish Posttraumatic Stress Disorder Checklist-Civilian version. Psychooncology. 2009 Jul. 9.
53. Kubzansky L D, Koenen K C. Is posttraumatic stress disorder related to development of heart disease? An update. Cleve Clin J Med. 2009 April; 76 Suppl 2:S60-5.
54. Kubzansky L D, Koenen K C, Jones C, Eaton W W. A prospective study of posttraumatic stress disorder symptoms and coronary heart disease in women. Health Psychol. 2009 January; 28(1):125-30.
55 Altemus M, Cloitre M, Dhabhar F S. Enhanced cellular immune response in women with PTSD related to childhood abuse. Am J Psychiatry. 2003 September; 160(9):1705-7.
56 Qureshi S U, Pyne J M, Magruder K M, Schulz P E, Kunik M E. The link between post-traumatic stress disorder and physical comorbidities: a systematic review. Psychiatr Q. 2009 June; 80(2):87-97.
57. el-Serag H B, Kunik M, Richardson P, Rabeneck L. Psychiatric disorders among veterans with hepatitis C infection. Gastroenterology. 2002 August; 123(2):476-82.
58. Sherbourne C D, Asch S M, Shugarman L R, Goebel J R, Lanto A B, Rubenstein L V, Wen L, Zubkoff L, Lorenz K A. Early identification of co-occurring pain, depression and anxiety. J Gen Intern Med. 2009 May; 24(5):620-5.
59. Kao A Y, Munandar R, Ferrara S L, Systrom D M, Sheridan R L, Cash S S, Ryan ET. Case records of the Massachusetts General Hospital. Case 19-2005. A 17-year-old girl with respiratory distress and hemiparesis after surviving a tsunami. N Engl J. Med. 2005 Jun. 23; 352(25):2628-36.
60. Brackbill R M, Nadler J L, DiGrande L, Ekenga C C, Farfel M R, Friedman S, Perlman S E, Stellman S D, Walker D J, Wu D, Yu S, Thorpe L E. Asthma and posttraumatic stress symptoms 5 to 6 years following exposure to the World Trade Center terrorist attack.
61 Afari N, Harder L H, Madra N J, Heppner P S, Moeller-Bertram T, King C, Baker DG. PTSD, combat injury, and headache in Veterans Returning from Iraq/Afghanistan. Headache. 2009 October; 49(9):1267-76.
62 Yehuda R, Bierer L M, Sarapas C, Makotkine I, Andrew R, Seckl J R. Cortisol metabolic predictors of response to psychotherapy for symptoms of PTSD in survivors of the World Trade Center attacks on Sep. 11, 2001. Psychoneuroendocrinology. 2009 October; 34(9):1304-13.
63. Pietrzak R H, Johnson D C, Goldstein M B, Malley J C, Southwick S M. Posttraumatic stress disorder mediates the relationship between mild traumatic brain injury and health and psychosocial functioning in veterans of Operations Enduring Freedom and Iraqi Freedom. J Nerv Ment Dis. 2009 October; 197(10):748-53.
64 Yon L, Kanchanapangka S, Chaiyabutr N, Stanczyk F, Meepan S, Lasley B. ACTH stimulation in four Asian bull elephants (Elephas maximus): an investigation of androgen sources in bull elephants. Gen Comp Endocrinol. 2007 May 1; 151(3):246-51.
65. Loria R M. Beta-Androstenes and Resistance to Viral and Bacterial Infections. Neuroimmunomodulation 2009; 16:88-95.
66 Yon L, Kanchanapangka S, Chaiyabutr N, Meepan S, Stanczyk F Z, Dahl N, Lasley B. A longitudinal study of LH, gonadal and adrenal steroids in four intact Asian bull elephants (Elephas maximus) and one castrate African bull (Loxodonta africana) during musth and non-musth periods. Gen Comp Endocrinol. 2007 May 1; 151(3):241-5.
67. Handa R J, Weiser M J, Zuloaga D G. A role for the androgen metabolite, 5alpha-androstane-3beta,17beta-diol, in modulating oestrogen receptor beta-mediated regulation of hormonal stress reactivity. J Neuroendocrinol. 2009 March; 21(4):351-8.
68. Handa R J, Pak T R, Kudwa A E, Lund T D, Hinds L. An alternate pathway for androgen regulation of brain function: activation of estrogen receptor beta by the metabolite of dihydrotestosterone, 5alpha-androstane-3beta,17beta-diol. Horm Behav. 2008 May; 53(5):741-52.
69. Lund T D, Hinds L R, Handa R J. The androgen 5alpha-dihydrotestosterone and its metabolite 5alpha-androstan-3beta, 17beta-diol inhibit the hypothalamo-pituitary-adrenal response to stress by acting through estrogen receptor beta-expressing neurons in the hypothalamus. J Neurosci. 2006 Feb. 1; 26(5):1448-56.
70 Jellinck P H, Kaufmann M, Gottfried-Blackmore A, McEwen B S, Jones G, Bulloch K. Selective conversion by microglia of dehydroepiandrosterone to 5-androstenediol-A steroid with inherent estrogenic properties. J Steroid Biochem Mol. Biol. 2007 November-December; 107(3-5):156-62.
71 Zhang T, Pan W, Takebe M, Schofield B, Sampson H, Li X M. Therapeutic effects of a fermented soy product on peanut hypersensitivity is associated with modulation of T-helper type 1 and T-helper type 2 responses. Clin Exp Allergy. 2008 November; 38(11):1808-18.
72. Jacob U, A Gocan, D Bachg, U Rohr. Applikation von fermentierter Soja bei Krebspatienten zur Verminderung von Kachexie and Erhöhung der Apoptose—eine prospektive Pilotstudie. J Gynäkol Endokrinol 2009; 16 (2): 52-61.
73 James K, Premchand N, Skibinska A, Skibinski G, Nicol M, Mason J I. IL-6, DHEA and the ageing process. Mech Ageing Dev 1997 February; 93(1-3):15-24
74. Seruga B, Zhang H, Bernstein L J, Tannock I F. Cytokines and their relationship to the symptoms and outcome of cancer. Nat Rev Cancer. 2008 November; 8(11):887-99.
75. Myint A M, Leonard B E, Steinbusch H W, Kim Y K. Th1, Th2, and Th3 cytokine alterations in major depression. J Affect Disord. 2005 October; 88(2):167-73.
76 Tagawa N, Hidaka Y, Takano T, Shimaoka Y, Kobayashi Y, Amino N. Serum concentrations of androstenediol and androstenediol sulfate, and their relation to cytokine production during and after normal pregnancy. Steroids. 2004 September; 69(10):675-80.
77. Maes M, Yirmyia R, Noraberg J, Brene S, Hibbeln J, Perini G, Kubera M, Bob P, Lerer B, Maj M. The inflammatory & neurodegenerative (I&ND) hypothesis of depression: leads for future research and new drug developments in depression. Metab Brain Dis. 2009 March; 24(1):27-53.
78. Chertkow Y, Weinreb O, Youdim M B, Silver H. Gene expression changes in peripheral mononuclear cells from schizophrenic patients treated with a combination of antipsychotic with fluvoxamine. Prog Neuropsychopharmacol Biot Psychiatry. 2007 Oct. 1; 31(7):1356-62.
79. Leslie D L, Kozma L, Martin A, Landeros A, Katsovich L, King R A, Leckman J F. Neuropsychiatric disorders associated with streptococcal infection: a case-control study among privately insured children. J Am Acad Child Adolesc Psychiatry. 2008 October; 47(10):1166-72.

1. A method where a severe mental disease (like schizophrenia, Bipolar, post traumatic stress disorder) are “simultaneously together treated” with accompanying severe diseases like metabolic disease, viral infections, and likewise where a specially fermented soy formulation (FSWW08) is employed.

2. A method for treating a person with a severe mental disorder, like anxiety disorder, bipolar disorder, schizophrenia, borderline personality disorder, ADHD, major depressive disorder, burnout, chronic fatigue syndrome, premenstrual dysphoric disorder, fibromyalgia, or postnatal depression, where hormones like DHEA, pregnenolone, are decreased and androstanediol, androstenediol, testosterone, estradiol are simultaneously increased by a natural or synthetic product or metabolite containing fermented soy or any other isoflavone or isoflavone derivative alone or in combination with the BOWMAN BIRK INHIBITORY FACTOR.

3. The method of claim 2 where the isoflavone concentration is higher than 300 mg/day in a man or a women, preferably between 500 mg to 1000 mg per day.

4. The method of claim 2 where the source of isoflavone is either natural, semi-natural or fully synthetic or even obtained by metabolism in the body.

5. The method of claim 2 where the source of isoflavones is either in an isolated form, in a fermented form, or in any other mixture of natural origin like alfalfa, red clover or likewise.

6. The method of claim 2 for a person in need where the isoflavone is containing isoflavones, curcumin or any other derivative from rhizome curcuma.

7. The method of claim 2 for a person in need where the isoflavone formulation is combined with other plant derived compounds altering NF-kB, like clover, oregano, thyme, walnuts, tomato extract, and coffee or likewise.

8. The method of claim 6 for treating a person with anxiety disorder, bipolar disorder, schizophrenia, borderline personality disorder, ADHD, major depressive disorder, burnout, chronic fatigue syndrome, premenstrual dysphoric disorder, fibromyalgia, treating simultaneously inflammation of the gastro intestinal tract, or lung and/or infection, or colitis ulcerosa in a subject in need thereof comprising administering to the subject a therapeutically effective amount of isoflavones or other plant products containing isoflavones, curcumin or any other derivative from rhizome curcuma.

9. The method of claim 6 for treating anxiety disorder, bipolar disorder, schizophrenia, borderline personality disorder, major depressive disorder, burnout, chronic fatigue syndrome, premenstrual dysphoric disorder, fibromyalgia, at the same time correcting inflammation like gastritis or lung inflammation and/or infection of a human in need thereof comprising administering a therapeutically effective amount of isoflavones combined with other plant products containing isoflavones, curcumin or any other derivative from rhizome curcuma.

10-19. (canceled)

20. A method where a mental disease like postnatal depression or postnatal stress is diagnosed or showing sign of underlying signs have been diagnosed in need thereof comprising administering to the mother a therapeutically effective amount of a fermented soy product.

21-33. (canceled)

34. The method of claim 2, wherein the formulation contains from soy bean hydrolyzed amino acids, isoflavones, protease inhibitors, saponins, phytosterols, inisotol hexaphosphate, polysaccharides, peptides and proteins fermentation derivatives.

35-82. (canceled)
